(19) United States
(12) Patent Application Publication
(10) Pub. No.: US 2006/0178398 A1
(43) Pub. Date: Aug. 10, 2006

(54) Therapeutic compounds for treating dyslipidemic conditions

(76) Inventors: Alan D. Adams, Cranford, NJ (US); Shaei Y. Huang, Lansdale, PA (US); Jason W. Szewczyk, New York, NJ (US)

Correspondence Address: MERCK AND CO., INC, P O BOX 2000, RAHWAY, NJ, 07065-0907, US

(21) Appl. No.: 11/226,782

(22) Filed: Sep. 14, 2005

Related U.S. Application Data
(60) Provisional application No. 60/610,518, filed on Sep. 16, 2004.

(51) Int. Cl. A61K 31/4709; A61K 31/4439; A61K 31/427; A61K 31/42

(52) U.S. Cl. 514/312; 514/314; 514/359; 514/379; 546/157; 546/158; 548/241; 548/181; 514/338; 514/365

(57) ABSTRACT

The present invention relates to novel LXR ligands of Formula I 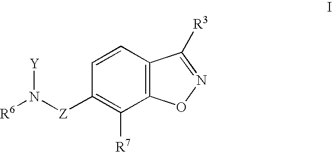 and pharmaceutically acceptable salts, esters and tautomers thereof, which are useful in the treatment of dyslipidemic conditions, particularly depressed levels of HDL cholesterol.

### BACKGROUND OF THE INVENTION

Recent publications in Nature Genetics, August, 1999 (Young et al., page 316; Bodzioch et al., page 347; Brooks-Wilson et al., page 335, and Rust et al., page 352) showed that humans with mutations in the gene ABCA1 (also previously known in the art as ABC1) have low levels of high density lipoprotein (HDL). Low HDL levels are a risk factor for atherosclerosis, myocardial infarction and related conditions such as ischemic stroke. Therefore, increasing the expression of the ABCA1 gene would be expected to increase HDL levels and decrease the occurrence of atherosclerosis, myocardial infarction and related conditions such as ischemic stroke. It has been reported that expression of the ABCA1 gene is increased by cholesterol loading of cells (Langmann et al., Biochem. Biophys. Res. Comm., 257, 29-33 (1999)). LXRα is a nuclear receptor that is required for the induction of cholesterol 7α-hydroxylase in mouse liver following cholesterol feeding (Peet et al., Cell, 93, 693-704 (1998)). LXRα (NR1H3) (for a unified system of nomenclature for the nuclear receptor superfamily see Cell 97, 161-163, 1999) and LXRβ (NR1H2) are activated by 22-(R)-hydroxycholesterol and other oxysterols (Janowski et al. Proc. Natl. Acad. Sci USA, 96, 266-271 (1999), Thomas A. Spencer et al. J. Med. Chem., 44, 886-897, (2001)). Some non-steroidal small molecule agonists of LXRα and LXRβ have been reported to affect circulating HDL levels, cholesterol absorption, reverse cholesterol transport and ABCA1 expression in vivo (Jon L. Collins et al J. Med. Chem 45, 1963-1966, (2002); J. R. Schultz, et al. Genes & Devel. 14, 2831-2838, (2000), J. J. Repa et al. Science, 289, 1524-1529, (2000)). A small molecule agonist of LXR has also been demonstrated to inhibit the development of atherosclerosis in a rodent model (Sean B. Joseph et al. PNAS 99, 7604-7609, (2002)). It has been found that LXRα and/or LXRβ cause the induction or regulation of ABCA1 expression, and that small molecule ligands of LXR are useful as drugs to increase the expression of ABCA1, increase levels of HDL and thereby decrease the risk of atherosclerosis, myocardial infarction and related conditions such as peripheral vascular disease and ischemic stroke.

The various dyslipidemic conditions, which are risk factors for atherosclerosis, are currently treated with several different classes of drugs, such as statins which are HMG-CoA reductase inhibitors, bile acid sequestrants (e.g., cholestyramine and colestipol), nicotinic acid (niacin), and fibrates. However, except for niacin, most of these treatments do not raise HDL as their primary effect. With favorable outcomes in many human studies, the statin class of drugs is used to modulate LDL and, to a lesser extent, HDL and triglycerides. Conditions principally characterized by elevated plasma triglycerides and low HDL are frequently treated with drugs belonging to the fibrate class. The fibrates are PPAR alpha agonists that lower triglycerides and raise HDL in many instances. There are no currently marketed drugs whose principal actions are mediated by LXR.

We have now discovered a new class of small molecules which are LXR ligands, i.e., LXRα and/or LXRβ ligands, and are therefore expected to be useful for modulation of HDL levels, ABCA1 gene expression and reverse cholesterol transport. The instant compounds have been shown to raise plasma levels of HDL in animal models and to increase cholesterol efflux from cells in vitro. These biological activities are critical for reverse cholesterol transport.

The novel compounds of this invention are intended as a treatment for dyslipidemias, especially low plasma HDL cholesterol levels, as well as for treatment and/or prevention of lipid accumulation in atherosclerotic plaques, which is an underlying cause or aggravating factor in atherosclerosis.

### SUMMARY OF THE INVENTION

The novel LXR ligands of the instant invention are compounds having Formula I 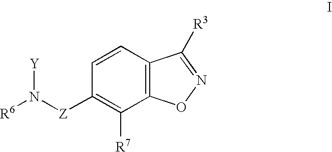
and pharmaceutically acceptable salts, esters and tautomers thereof, wherein
Y has one of the following structures: 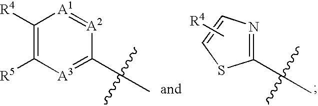
wherein A¹, A², and A³ are each independently selected from the group consisting of —C(R¹)═ and —N═, wherein one or both of A¹ and A² is —C(R¹)═;
R¹ is selected from —H, halogen, —C₁-C₆alkyl and —OC₁-C₆alkyl, wherein —C₁-C₆alkyl and —OC₁-C₆alkyl are each optionally substituted with 1-3 halogens;
R⁶ is C₁-C₄alkyl;
or alternatively Y and R⁶ taken together form a bicyclic heterocyclic ring selected from the following group: 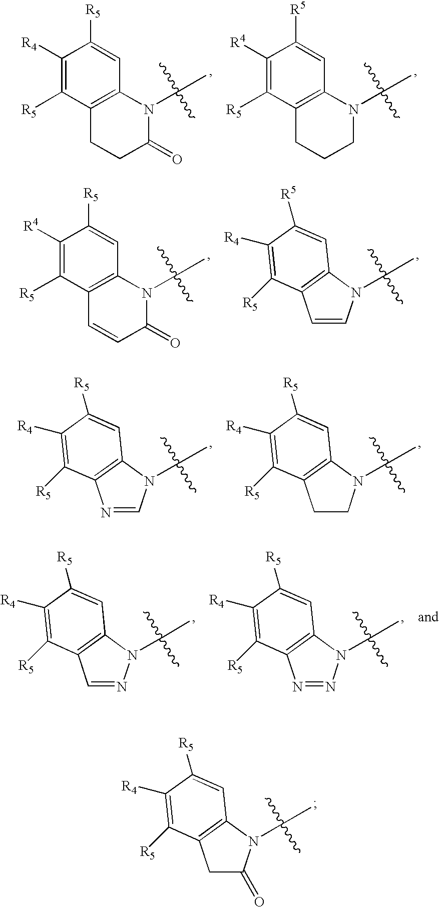
Z is a difunctional connecting moiety selected from the group consisting of C₁-C₄alkylene and —C₁-C₃alkyleneO—, wherein the O of —C₁-C₃alkyleneO— is connected to the benzisoxazole ring in Formula I;
R³ is —C₁-C₆alkyl, which is optionally substituted with 1-3 halogens;
R⁴ is selected from the group consisting of —CO₂R² and —C₁-C₄alkylCO₂R², with the proviso that when A¹, A² and A³ are all equal to —C(R¹)═ and R⁶ is C₁-C₄alkyl, so that Y is a phenyl ring that is not fused to another ring by being joined with R⁶, then R⁴ is —CO₂R²;
R² is selected from the group consisting of H and C₁-C₄alkyl;
R⁵ is selected from the group consisting of H, halogen, C₁-C₅alkyl, and —OC₁-C₅alkyl; and
R⁷ is —C₁-C₆ alkyl, which is optionally substituted with 1-3 halogens.

In the structural formulae above and elsewhere in this application, alkyl groups can be either linear or branched, unless otherwise stated.

Compounds of Formula I are novel LXR ligands and are useful in the treatment of dyslipidemic conditions such as low levels of HDL cholesterol.

One object of the instant invention is to provide a method for treating depressed plasma HDL cholesterol levels comprising administering a therapeutically effective amount of a compound of Formula I to a patient in need of such treatment.

Another object is to provide a method for preventing or treating dyslipidemic conditions comprising administering a prophylactically or therapeutically effective amount, as appropriate, of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. The method of this invention also serves to remove cholesterol from tissue deposits such as xanthomas and atherosclerotic lesions by hastening the efflux of cholesterol from cells in those lesions.

Another object of the present invention is the use of the compounds of the present invention for the manufacture of a medicament useful in treating, preventing or reducing the risk of developing these conditions.

Other objects of this invention are to provide processes for making the compounds of Formula I and to provide novel pharmaceutical compositions comprising these compounds. Additional objects will be evident from the following detailed description.

### DETAILED DESCRIPTION OF THE INVENTION

One embodiment of the invention comprises compounds of Formula I in which Y is selected from: 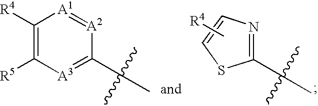
in which A¹, A², and A³ are each independently selected from the group consisting of —C(R¹)═ and —N═, wherein one or both of A¹ and A² is —C(R¹)═;
R¹ is selected from the group consisting of —H, halogen, —CH₃, —CF₃, —OCH₃, and —OCF₃;
R⁶is C₁-C₄alkyl;
Z is —C₁-C₃alkyleneO—, wherein the O of —C₁-C₃alkyleneO— is connected to the benzisoxazole ring in Formula I;
R³ is —C₁-C₆alkyl, which is optionally substituted with 1-3 halogens;
R⁴ is selected from the group consisting of —CO₂R² and —C₁-C₄alkylCO₂R², with the proviso that when A¹, A² and A³ are all equal to —C(R¹)═ and R⁶ is C₁-C₄alkyl, so that Y is a phenyl ring that is not fused to another ring, then R⁴ is —CO₂R²;
R² is selected from the group consisting of H and C₁-C₄alkyl;
R⁵ is selected from the group consisting of H, halogen, —CH₃, and —OCH₃; and
R⁷ is —C₁-C₆alkyl, which is optionally substituted with 1-3 halogens.

In subgroups of the above compounds, Y is 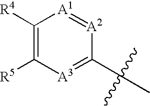
wherein A¹, A², and A³ are each independently selected from the group consisting of —C(R¹)═ and —N═, wherein one or both of A¹ and A² is —C(R¹)═;
R¹ is selected from the group consisting of —H, halogen, —CH₃, —CF₃, —OCH₃, and —OCF₃;
R⁶is CH₃;
Z is —CH₂CH₂CH₂O—, wherein the —O— of —CH₂CH₂CH₂O— is connected to the benzisoxazole ring in Formula I;
R³ is —CF₃;
R⁴ is selected from the group consisting of —CO₂H and —C₁-C₄alkylCO₂H, with the proviso that when A¹, A² and A³ are all equal to —C(R¹)═, so that Y is a phenyl ring, then R⁴ is —CO₂H;
R⁵ is selected from the group consisting of H, halogen, —CH₃, and —OCH₃; and
R⁷ is —CH₂CH₂CH₃.

A subgroup of the compounds described above comprise structures in which A¹, A², and A³ are all —C(R¹)═.

Another subgroup of the above compounds comprises structures in which A¹ and A³ are each —N═ and A² is —C(R¹)═.

Another subgroup of the above compounds comprises structures in which A² and A³ are —N═ and A¹ is —C(R¹)═.

Another subgroup of the above compounds comprises structures in which A¹ and A³ are —C(R¹)═ and A² is —N═.

Another subgroup of the above compounds comprises structures in which A² and A³ are each —C(R¹)═ and A¹ is —N═.

Another subgroup of the above compounds comprises structures in which A¹ and A² are each —C(R¹)═ and A³ is —N═.

Other subgroups of the structures described above comprise compounds of Formula I in which Y is 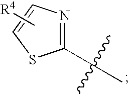
R⁶ is C₁-C₄alkyl;
Z is —C₁-C₃alkyleneO—, wherein the O of —C₁-C₃alkyleneO— is connected to the benzisoxazole ring in Formula I;
R³ is —C₁-C₆ alkyl, which is optionally substituted with 1-3 halogens;
R⁴ is selected from the group consisting of —CO₂R² and —C₁-C₄alkylCO₂R²;
R² is selected from the group consisting of H and C₁-C₄alkyl; and
R⁷ is —C₁-C₆alkyl, which is optionally substituted with 1-3 halogens.

In preferred subgroups of the compounds of Formula I in which Y is 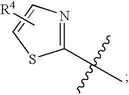
R⁶ is CH₃;
Z is —CH₂CH₂CH₂O—, wherein the —O— of —CH₂CH₂CH₂O— is connected to the benzisoxazole ring in Formula I;
R³ is —CF₃;
R⁴ is selected from the group consisting of —CO₂H and —C₁-C₄alkylCO₂H; and R⁷ is —CH₂CH₂CH₃.

Many preferred embodiments of the invention comprise compounds having the Formula Ia: 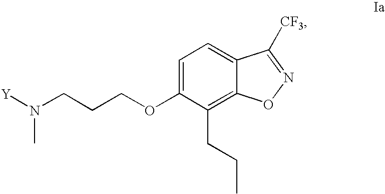
wherein Y is as described above.

Other embodiments of the invention comprise compounds having Formula I, including pharmaceutically acceptable salts, esters and tautomers thereof, in which Y and R⁶ taken together form a bicyclic heterocyclic ring which has one of the following structures: 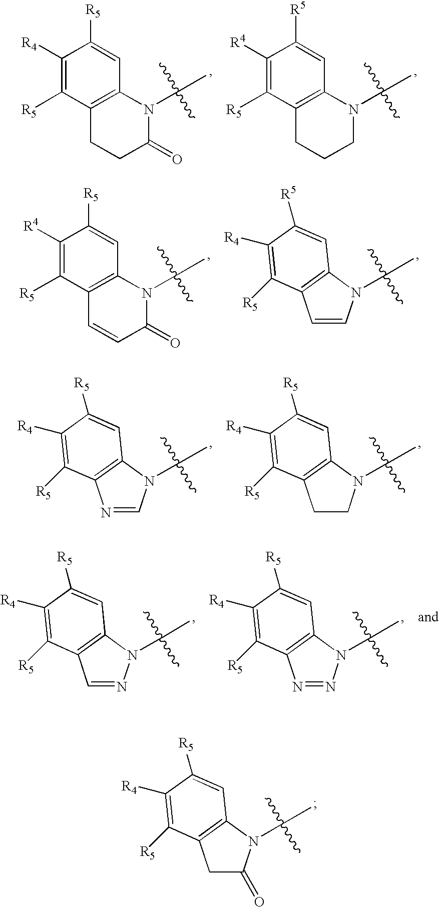
wherein Z is a difunctional connecting moiety selected from the group consisting of C₁-C₄alkylene and —C₁-C₃alkyleneO—, wherein the O of —C₁-C₃alkyleneO— is connected to the benzisoxazole ring in Formula I;
R³ is —C₁-C₆alkyl, which is optionally substituted with 1-3 halogens;
R⁴ is selected from the group consisting of —CO₂R² and —C₁-C₄alkylCO₂R²;
R² is selected from the group consisting of H and C₁-C₄alkyl;
R⁵ is selected from the group consisting of H, halogen, —CH₃, and —OCH₃; and
R⁷ is —C₁-C₆ alkyl, which is optionally substituted with 1-3 halogens.

In preferred subgroups of the compounds described above,
Z is —CH₂CH₂CH₂O—, wherein the —O— of —CH₂CH₂CH₂O— is connected to the benzisoxazole ring in Formula I;
R³ is —CF₃;
R⁴ is selected from the group consisting of —CO₂H and —C₁-C₄alkylCO₂H;
R⁵ is selected from the group consisting of H, halogen, —CH₃, and —OCH₃; and
R⁷ is —CH₂CH₂CH₃.

In other subgroups of the above compounds, R⁴ is —CO₂H or —CH₂CO₂H.

In general, in many preferred compounds having Formula I, R³ is CF₃ or —CH₂C(CH₃)₃. Most preferably, R³ is CF₃.

In many preferred compounds having Formula I, R² is H.

In many preferred compounds having Formula I, R⁷ is n-C₃H₇.

Specific examples of compounds of Formula I are named below:
(1) 1-(3-{[7-propyl-3-(trifluoromethyl)-1,2-benzisoxazol-6-yl]oxy}propyl)-1H-indole-5-carboxylic acid,
(2) 4-[methyl(3-{[7-propyl-3-(trifluoromethyl)-1,2-benzisoxazol-6-yl]oxy}propyl)amino]benzoic acid,
(3) 3-methyl-4-[methyl(3-{[7-propyl-3-(trifluoromethyl)-1,2-benzisoxazol-6-yl]oxy}propyl)amino]benzoic acid,
(4) [2-oxo-1-(3-{[7-propyl-3-(trifluoromethyl)-1,2-benzisoxazol-6-yl]oxy}propyl)-1,2,3,4-tetrahydroquinolin-6-yl]acetic acid,
(5) 1-(3-{[7-propyl-3-(trifluoromethyl)-1,2-benzisoxazol-6-yl]oxy}propyl)-1,2,3,4-tetrahydroquinoline-6-carboxylic acid,
(6) [1-(3-{[7-propyl-3-(trifluoromethyl)-1,2-benzisoxazol-6-yl]oxy}propyl)-1,2,3,4-tetrahydroquinolin-6-yl]acetic acid,
(7) 3-chloro-4-[methyl(3-{[7-propyl-3-(trifluoromethyl)-1,2-benzisoxazol-6-yl]oxy}propyl)amino]benzoic acid,
(8) 2-chloro-4-[methyl(3-{[7-propyl-3-(trifluoromethyl)-1,2-benzisoxazol-6-yl]oxy}propyl)amino]benzoic acid,
(9) 4-[methyl(3-{[7-propyl-3-(trifluoromethyl)-1,2-benzisoxazol-6-yl]oxy}propyl)amino]-3-(trifluoromethoxy)benzoic acid,
(10) [2-oxo-1-(3-{[7-propyl-3-(trifluoromethyl)-1,2-benzisoxazol-6-yl]oxy}propyl)-1,2-dihydroquinolin-6-yl]acetic acid,
(11) 2-oxo-1-(3-{[7-propyl-3-(trifluoromethyl)-1,2-benzisoxazol-6-yl]oxy}propyl)indoline-5-carboxylic acid,
(12) 2-methoxy-4-[methyl(3-{[7-propyl-3-(trifluoromethyl)-1,2-benzisoxazol-6-yl]oxy}propyl)amino]benzoic acid,
(13) 4-[methyl(3-{[7-propyl-3-(trifluoromethyl)-1,2-benzisoxazol-6-yl]oxy}propyl)amino]-2-(trifluoromethyl)benzoic acid,
(14) 2-fluoro-4-[methyl(3-{[7-propyl-3-(trifluoromethyl)-1,2-benzisoxazol-6-yl]oxy}propyl)amino]benzoic acid,
(15) 2-bromo-4-[methyl(3-{[7-propyl-3-(trifluoromethyl)-1,2-benzisoxazol-6-yl]oxy}propyl)amino]benzoic acid,
(16) 6-[methyl(3-{[7-propyl-3-(trifluoromethyl)-1,2-benzisoxazol-6-yl]oxy}propyl)amino]nicotinic acid,
(17) {6-[methyl(3-{[7-propyl-3-(trifluoromethyl)-1,2-benzisoxazol-6-yl]oxy}propyl)amino]pyridin-3-yl}acetic acid,
(18) 5-[methyl(3-{[7-propyl-3-(trifluoromethyl)-1,2-benzisoxazol-6-yl]oxy}propyl)amino]pyrazine-2-carboxylic acid,
(19) {5-[methyl(3-{[7-propyl-3-(trifluoromethyl)-1,2-benzisoxazol-6-yl]oxy}propyl)amino]pyrazin-2-yl}acetic acid,
(20) 2-[methyl(3-{[7-propyl-3-(trifluoromethyl)-1,2-benzisoxazol-6-yl]oxy}propyl)amino]pyrimidine-5-carboxylic acid,
(21) {2-[methyl(3-{[7-propyl-3-(trifluoromethyl)-1,2-benzisoxazol-6-yl]oxy}propyl)amino]pyrimidin-5-yl}acetic acid,
(22) {2-[methyl(3-{[7-propyl-3-(trifluoromethyl)-1,2-benzisoxazol-6-yl]oxy}propyl)amino]-1,3-thiazol-4-yl}acetic acid,
(23) 2-[methyl(3-{[7-propyl-3-(trifluoromethyl)-1,2-benzisoxazol-6-yl]oxy}propyl)amino]-1,3-thiazole-5-carboxylic acid,
(24) {2-[methyl(3-{[7-propyl-3-(trifluoromethyl)-1,2-benzisoxazol-6-yl]oxy}propyl)amino]-1,3-thiazol-5-yl}acetic acid,
(25) [1-(3-{[7-propyl-3-(trifluoromethyl)-1,2-benzisoxazol-6-yl]oxy}propyl)-1H-indol-5-yl]acetic acid,
(26) [1-(3-{[7-propyl-3-(trifluoromethyl)-1,2-benzisoxazol-6-yl]oxy}propyl)-1H-benzimidazol-5-yl]acetic acid,
(27) 2-methyl-2-[1-(3-{[7-propyl-3-(trifluoromethyl)-1,2-benzisoxazol-6-yl]oxy}propyl)-1H-indol-5-yl]propanoic acid,
(28) 2-[1-(3-{[7-propyl-3-(trifluoromethyl)-1,2-benzisoxazol-6-yl]oxy}propyl)-1H-indol-5-yl]propanoic acid,
(29) [1-(3-{[7-propyl-3-(trifluoromethyl)-1,2-benzisoxazol-6-yl]oxy}propyl)-1H-1,2,3-benzotriazol-5-yl]acetic acid,
(30) [2-oxo-1-(3-{[7-propyl-3-(trifluoromethyl)-1,2-benzisoxazol-6-yl]oxy}propyl)-2,3-dihydro-1H-indol-5-yl]acetic acid
(31) [1-(3-{[7-propyl-3-(trifluoromethyl)-1,2-benzisoxazol-6-yl]oxy}propyl)-1H-indazol-5-yl]acetic acid,
(32) 6-chloro-1-(3-{[7-propyl-3-(trifluoromethyl)-1,2-benzisoxazol-6-yl]oxy}propyl)-1H-indole-5-carboxylic acid,
(33) 4-chloro-1-(3-{[7-propyl-3-(trifluoromethyl)-1,2-benzisoxazol-6-yl]oxy}propyl)-1H-indole-5-carboxylic acid,
(34) [6-chloro-1-(3-{[7-propyl-3-(trifluoromethyl)-1,2-benzisoxazol-6-yl]oxy}propyl)-1H-indol-5-yl]acetic acid,
(35) [6-chloro-2-oxo-1-(3-{[7-propyl-3-(trifluoromethyl)-1,2-benzisoxazol-6-yl]oxy}propyl)-2,3-dihydro-1H-indol-5-yl]acetic acid,
(36) [4-chloro-1-(3-{[7-propyl-3-(trifluoromethyl)-1,2-benzisoxazol-6-yl]oxy}propyl)-1H-indol-5-yl]acetic acid,
(37) 6-methoxy-1-(3-{[7-propyl-3-(trifluoromethyl)-1,2-benzisoxazol-6-yl]oxy}propyl)-1H-indole-5-carboxylic acid, and
(38) 3-[1-(3-{[7-propyl-3-(trifluoromethyl)-1,2-benzisoxazol-6-yl]oxy}propyl)-1H-indol-5-yl]propanoic acid; and pharmaceutically acceptable salts thereof.

### DEFINITIONS

As used herein “alkyl” is intended to include both branched- and straight-chain saturated aliphatic hydrocarbon groups having the specified number of carbon atoms. Examples of alkyl groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), isopentyl, isohexyl and the like. The term “C₁₋₆alkyl”, as used herein, refers to branched- and straight-chain saturated aliphatic hydrocarbon groups having 1-6 carbon atoms.

The term alkylene means that an alkyl moiety is a difunctional alkyl moiety. The term —C₁-C₃alkyleneO— means a difunctional moiety, where one point of attachment is a carbon of the alkylene and the other point of attachment is the oxygen. Alkylene groups can be linear or branched, unless otherwise specified.

The term “C₂₋₆alkenyl” as used herein, refers to a straight or branched 2-6 carbon chain with at least one carbon-carbon double bond. Examples of alkenyl include, but are not limited to, vinyl, allyl, isopropenyl, pentenyl, hexenyl, 1-propenyl, 2-butenyl, 2-methyl-2-butenyl, and the like.

“C₃₋₆cycloalkyl” means a monocyclic saturated carbocyclic ring, having from 3 to 6 carbon atoms, wherein one carbocyclic ring carbon is the point of attachment. Examples of cycloalkyl include, but are not limited to, cyclopropyl, cyclobutyl, cyclopentyl, cyclohexyl, and the like.

The terms “halo” and “halogen” are meant to include fluoro, chloro, bromo and iodo, unless otherwise noted. Fluoro and chloro are preferred.

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, di ethyl amine, piperazine, morpholine, 2,4,4-trimethyl-2-pentamine and tris(hydroxymethyl)aminomethane.

Examples of pharmaceutically acceptable esters include, but are not limited to, —C₁₋₄ alkyl and —C₁₋₄ alkyl substituted with phenyl-, dimethylamino-, and acetylamino. “C₁₋₄ alkyl” herein includes straight or branched aliphatic chains containing from 1 to 4 carbon atoms, for example methyl, ethyl, n-propyl, n-butyl, iso-propyl, sec-butyl and tert-butyl.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

When referring to moieties which may optionally be substituted herein, e.g., alkyl groups, cycloalkyl groups, phenyl groups, heterocycloalkyl groups, and the like, the phrases used herein “unsubstituted, mono- or disubstituted with a substituent independently selected at each occurrence from the group consisting of” and “unsubstituted, mono- or polysubstituted with a substituent independently selected at each occurrence from the group consisting of” are intended to mean that the total number of substituents on the moiety overall may be zero, one or more than one, and that each carbon and nitrogen atom available for substitution in the given moiety may independently be unsubstituted or mono- or poly-substituted, with one or more substituents that are the same or different at each occurrence and which result in the creation of a stable structure. The term “poly-substituted” is intended to mean two or more substituents, e.g. di-, tri-, tetra-, penta- substitution and higher as appropriate, valence and stability permitting.

In choosing compounds of the present invention, one of ordinary skill in the art will recognize that the various substituents, i.e. R¹, R², etc., are to be chosen in conformity with well-known principles of chemical structure connectivity and stability. When any variable (e.g., R¹, R², etc.) occurs more than one time in any constituent or in formula I, its definition on each occurrence is independent of its definition at every other occurrence. Also, combinations of substituents and/or variables are permissible only if such combinations result in stable compounds. Compounds of Formula I may contain one or more asymmetric centers and can thus occur as racemates and racemic mixtures, single enantiomers, enantiomeric mixtures, diastereomeric mixtures and individual diastereomers. The present invention is meant to comprehend all such isomeric forms of the compounds of Formula I. All such isomeric forms of the compounds of Formula I are included within the scope of this invention.

Some of the compounds described herein contain olefinic double bonds, and unless specified otherwise, are meant to include both E and Z geometric isomers.

The term “tautomers” embraces the standard meaning of the term, i.e. a type of isomerism in which two or more isomers are rapidly interconverted so that they ordinarily exist together in equilibrium. Tautomers include, e.g., compounds that undergo facile proton shifts from one atom of the compound to another atom of the compound. Some of the compounds described herein may exist as tautomers with different points of attachment of hydrogen. Such an example may be a ketone and its enol form known as keto-enol tautomers. The individual tautomers of the compounds of Formula I, as well as mixtures thereof, are included in the scope of this invention. By way of illustration, tautomers included in this definition include, but are not limited to:

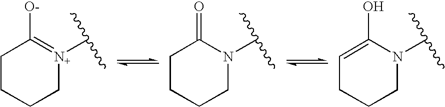

The term “rac” means racemic mixture, which is defined as a mixture comprised of equal amounts of enantiomers. If desired, racemic mixtures of compounds of Formula I may be separated by the coupling of a racemic mixture of the compounds of Formula I to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage and removal of the added chiral residue. The racemic mixture of the compounds of Formula I can also be separated directly by chromatographic methods utilizing chiral stationary phases, which methods are well known in the art.

Alternatively, any enantiomer of a compound of the general Formula I may be obtained by stereoselective synthesis using optically pure starting materials or reagents of known configuration. Such methods are well known in the art.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

Some abbreviations used herein are as follows: Ac is acetyl [CH₃C(O)—]; PG is protecting group; LG is leaving group; Ac₂O is acetic anhydride; 9-BBN is 9-borabicyclo[3.3.1]nonane; Pd(dba)₂ is tris(dibenzylideneacetone)dipalladium, PdCl₂dppf is dichlorobis-(triphenylphosphene) palladium, Ph is phenyl; PhMe is toluene; PPh₃ is triphenylphosphine; Bn is benzyl; Me is methyl, Et is ethyl, EtOH is ethanol, EtOAc is ethyl acetate, Et₃N is triethylamine, tBu is tert-butyl, PMB is para-methoxybenzyl; DMAP is 4-(dimethylamino)pyridine; DMF is N,N-dimethylformamide; DMSO is dimethyl sulfoxide; DIAD is diisopropylazodicarboxylate; Tf₂O is triflic anhydride, Tf is triflate, TBAF is tetrabutyl ammonium fluoride; THF is tetrahydrofuran; TMS is trimethylsilyl; TBS is tert-butyldimethylsilyl; CDI is 1,1′-carbonyldiimidazole; HOBt is 1-hydroxybenzotriazole; EDAC (or EDC) is 1-ethyl-3-[3-(dimethylamino)propyl]-carbodiimide HCl; HCl is hydrochloric acid; NaHMDS is sodium hexamethyldisiliazide; LiHMDS is lithium hexamethyldisiliazide; DIBAL is diisobutylaluminum hydride; TPAP is tetrapropylammonium perruthenate; NMO is N-methylmorpholine N oxide; MsCl is methanesulfonyl chloride; HPLC is high performance liquid chromatography; NaOAc is sodium acetate; NaOtBu is sodium tert-butoxide, TLC is thin layer chromatography; RT is room temperature; N is normal; mmol is millimole; M is molar; TFA is trifluoroacetic acid.

### GENERAL SCHEMES

The compounds of this invention can be prepared employing the following general procedures. Benzisoxazole intermediates may be prepared from commercially available or readily accessible resorcinols as shown in Scheme 1 or alternate synthetic pathways as reported in the literature. See for example: Shutske, G. M. et al. J. Med. Chem., 25 (1), 36, (1982); Poissonnet, G. Synth. Commun., 27 (22), 3839-3846, (1997); Crabbe, P. Villarino, A. Muchowski, J. M. J. Chem. Soc., Perkin Trans 1, 1973, 2220.

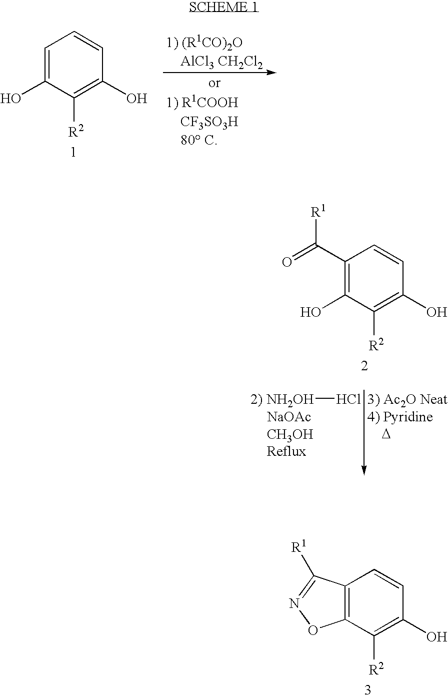

For maximum flexibility these phenolic benzisoxazoles (3) may be converted to intermediate halo-alkyl or amino-alkyl reagents for condensation with a variety of nucleophiles or electrophiles as shown in SCHEME 2 below. Formation of the secondary amine 7 typically occurs in the presence of a large excess of the amine partner. Coupling with a completely elaborated nucleophilic amine reagent with the desired R residues can lead directly to the desired compounds such as compound 6. Some nucleophilic partners may contain other protected functionality which will need to be elaborated, or some, such as an ester, will be de-protected in a final step. For cases where the amino-aryl reagent is not readily available, many classes of amino-aryl can be prepared by displacement of a halogen catalyzed by base or transition metal catalyst as for compound 9. The most favorable cases for this route are typified by the ortho-haloazaheterocycles as shown for compound 8 in SCHEME 2. 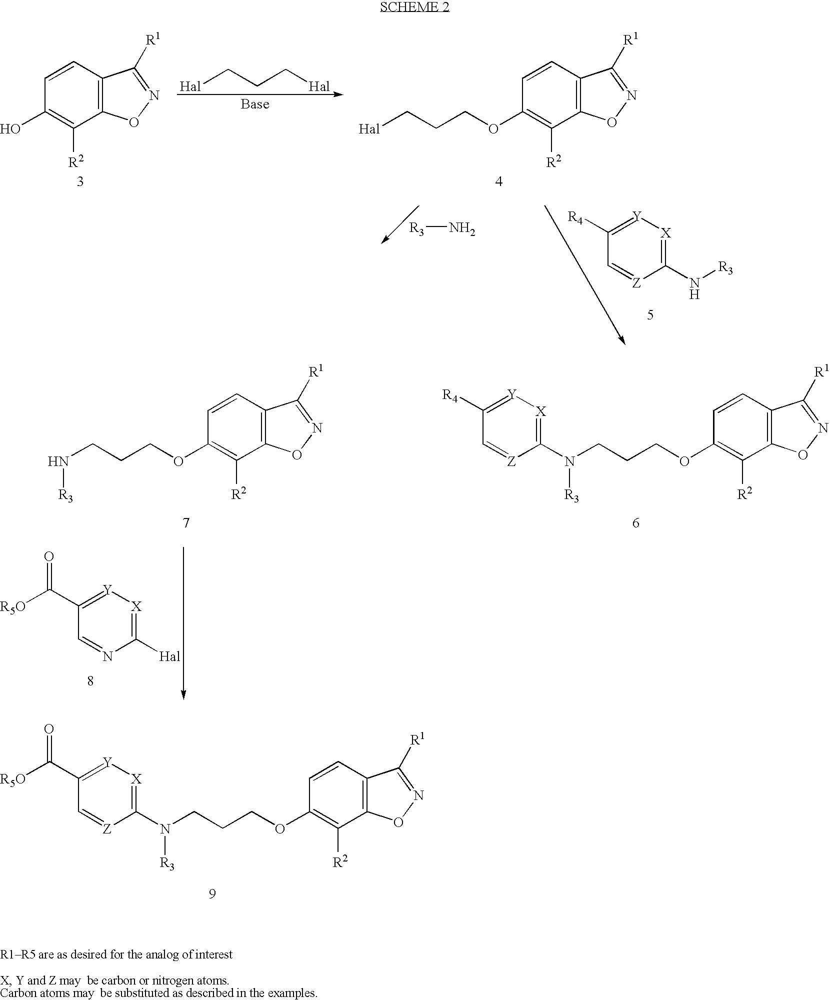
In cases where the amino residue is part of a fused bicyclic ring system, several routes may be used to access the bicyclic analogs. Preformed cyclic amines may be used in displacements to yield the alkylated bicyclic amines directly as for compound 11. Subsequent modifications of the resulting heterocycle can then provide access to further bicyclic analogs. Alternatively, a coupling partner may be used which carries a substituent which is used to construct the appended ring after coupling the partners, as in compound 12. 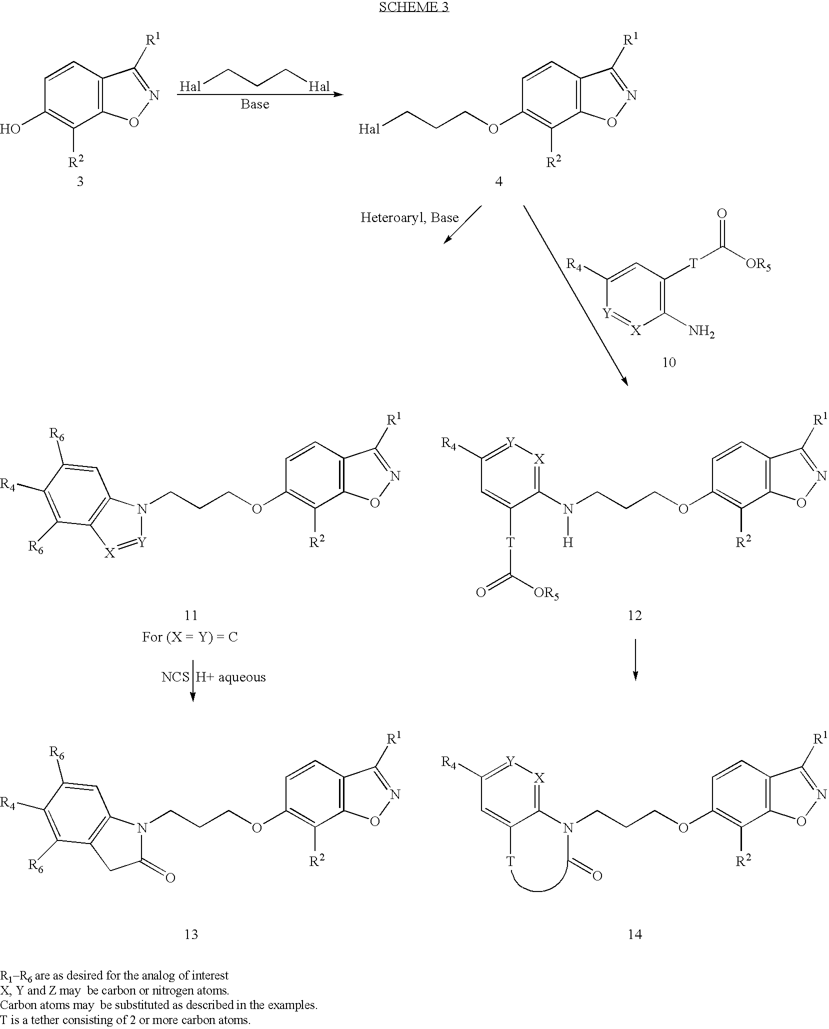
Some intermediate products will require elaboration of the initial condensation products to the desired final configurations. These modifications are well known in the art and are typified by the routes shown in SCHEME 4.

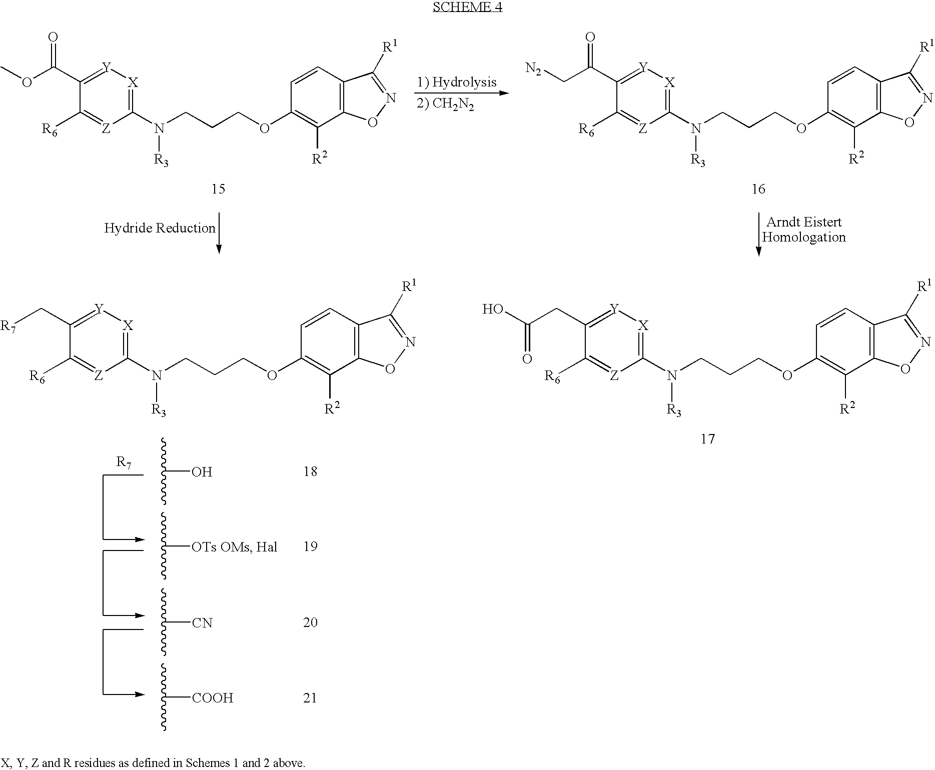

The instant invention provides methods for treating lipid disorders, particularly for treating below-desired plasma HDL cholesterol levels, as well as for treating and/or reducing the risk for diseases and conditions affected by LXR activity, comprising administering a therapeutically effective amount of a compound of Formula I to a person in need of such treatment. Any patient having a depressed plasma HDL cholesterol level, or desiring to increase their HDL cholesterol level may use this treatment. Particularly suitable patients in need of such treatment are those whose plasma HDL cholesterol level is depressed, i.e., below the clinically desirable level. Currently, the clinically desirable HDL cholesterol level is considered to be a minimum of 40 mg/dl in men and about 50 mg/dl or higher in women. NCEP guidelines define 60 mg/dl as a desirable, cardioprotective, HDL level.

The method of this invention also serves to prevent lipid accumulation in, or remove lipids from, tissue deposits such as atherosclerotic plaques or xanthomas in a patient with atherosclerotic disease manifested by clinical signs such as angina, claudication, bruits, one that has suffered a myocardial infarction or transient ischemic attack, or one diagnosed by angiography, sonography or MRI.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal, including a human, who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease (CHD) events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those with dyslipidemic conditions including depressed or below-desirable plasma levels of HDL cholesterol, as well as those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by reverse cholesterol transport.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective arnount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage amount of a compound of Formula I that a patient receives can be selected so as to achieve the amount of lipid level modification desired, particularly to achieve a desired level of HDL cholesterol. The dosage a patient receives may also be titrated over time in order to reach a target lipid profile. The dosage regimen utilizing a compound of Formula I is selected in accordance with a variety of factors including type, species, age, weight, sex and medical condition of the patient; the severity of the condition to be treated; the potency of the compound chosen to be administered; drug combinations; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

An effective amount of compound for use in the method of this invention is about 0.01 mg/kg to about 30 mg/kg of body weight per day, or about 0.7 mg to about 2100 mg per day for a 70 kg adult patient in single or divided doses per day. More particularly, examples of daily doses of a compound of this invention are 1, 2, 5, 10, 20, 50, 100, 250, 500, and 1000 mg per day, administered as a single dose or in divided doses 2-6 times per day, or in controlled release form. Dosage amounts will vary depending on factors as noted above, including the potency of the particular compound. Although the active drug of the present invention may be administered in divided doses, for example from one to four times daily, a single daily dose of the active drug is preferred.

The active drug employed in the instant therapy can be administered in such oral forms as tablets, capsules, pills, powders, granules, elixirs, tinctures, suspensions, syrups, and emulsions. Oral formulations are preferred.

Administration of the active drug can be via any pharmaceutically acceptable route and in any pharmaceutically acceptable dosage form. This includes the use of oral conventional rapid-release, time controlled-release and delayed-release (such as enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, microcrystalline cellulose, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents (e.g. croscarmellose sodium) and coloring and flavoring agents can also be incorporated into the mixture. Stabilizing agents such as antioxidants, for example butylated hydroxyanisole (BHA), 2,6-di-tert-butyl-4-methyl phenol (BHT), propyl gall ate, sodium ascorbate, citric acid, calcium metabisulphite, hydroquinone, and 7-hydroxycoumarin, can also be added to stabilize the dosage forms. Other suitable components include gelatin, sweeteners, natural and synthetic gums such as acacia, tragacanth or alginates, carboxymethylcellulose, polyethylene glycol, waxes and the like. Tablets may also be coated with a film.

An example of a tablet formulation comprising a 100 mg dose of a compound of Formula I as the API follows. The tablet is made by a direct compression process. A 100 mg potency tablet comprises 100 mg of the the compound, 130 mg of microcrystalline cellulose, 130 mg of mannitol (or 130 mg of dicalcium phosphate), 8 mg of croscarmellose sodium, 8 mg of magnesium stearate and 16 mg of Opadry White (a proprietary coating material made by Colorcon, West Point, Pa.). The API, microcrystalline cellulose, mannitol (or dicalcium phosphate), and croscarmellose sodium are first blended, and the mixture is then lubricated with magnesium stearate and pressed into tablets. The tablets are then film coated with Opadry White.

The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

In a broad embodiment, any suitable additional active agent or agents may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as ZD-4522, (CRESTOR®; see U.S. Pat. No. 5,260,440, and Drugs of the Future, 1999, 24(5), pp. 511-513); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; cholesteryl ester transfer protein (CETP) inhibitors, such as Pfizer's torcetrapib, CP 529,414 (WO/0038722 and EP 818448) and Pharmacia's SC-744 and SC-795; niacin; probucol; bile acid sequestrants; LDL (low density lipoprotein) receptor inducers; platelet aggregation inhibitors, for example glycoprotein IIb/IIa fibrinogen receptor antagonists and aspirin; human peroxisome proliferator activated receptor gamma (PPARγ) agonists including the compounds commonly referred to as glitazones for example pioglitazone, rosiglitazone, balaglitazone and netoglitazone, and, including those compounds included within the structural class known as thiazolidinediones as well as those PPARγ agonists outside the thiazolidinedione structural class, including PPAR gamma partial agonists such as those disclosed in WO 2004/020409 and WO 2004/019869; PPARα agonists such as clofibrate, fenofibrate including micronized fenofibrate, other fibrate class PPARα agonists and gemlibrozil; PPAR dual axy agonists including aryloxyacetic acids (see U.S. Pat. No. 6,569,879), 2-aryloxy-2-arylalkanoic acids (see WO02/064094), benzopyrancarboxylic acids (see U.S. Patent Publication No. 20020103242), muraglitazar, tesaglitazar, naveglitazar, and Tak-559; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelin antagonists; agents that enhance ABCA1 gene expression; FXR ligands including both inhibitors and agonists; bisphosphonate compounds such as alendronate sodium; and cyclooxygenase-2 inhibitors such as rofecoxib, celecoxib, and etoricoxib.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A-cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Patent 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 02/066464, WO 00/63703, WO /0060107, WO 00/38725, WO 00/34240, WO 00/20623, WO 97/45406, WO 97/16424, WO 97/16455, and WO 95/08532, the entire contents of all of which are hereby incorporated by reference.

An exemplary cholesterol absorption inhibitor is ezetimibe, also known as SCH-58235, which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966 and shown below as

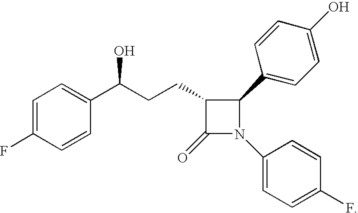

Additional exemplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula 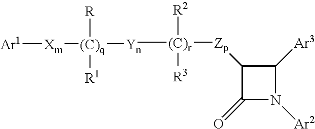
as defined in column 2, lines 20-63 (hereby incorporated by reference).

Additional exemplary C-glycosidic azetidinone cholesterol absorption inhibitors are disclosed in WO 02/066464 (hereby incorporated by reference in its entirety), represented by the formula 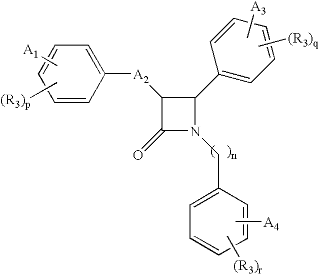
as defined on pages 3 line 24—page 5 line 3. These and other cholesterol absorption inhibitors can be identified according to the assay of hypolipidemic compounds using the hyperlipidemic hamster described in U.S. Pat. No. 5,767,115, column 19, lines 47-65 (hereby incorporated by reference), in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

According to a further aspect of the present invention there is provided the use of a compound of Formula I for the manufacture of a medicament for the treatment, prevention, or reduction in risk of developing a LXR receptor mediated disease. A therapeutically or prophylactically effective amount, as appropriate, of a compound of Formula I can be used for the preparation of a medicament useful for treating lipid disorders, particularly for treating depressed HDL cholesterol levels as well as for treating and/or reducing the risk for diseases and conditions affected by agonism of LXR and affected by reverse cholesterol transport, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 0.7 mg to about 2100 mg of a compound of Formula I, or more particularly about 7 mg to about 1050 mg. The medicament comprised of a compound of Formula I may also be prepared with one or more additional active agents, such as those described supra.

As used herein, the term LXR includes all subtypes of this receptor, e.g., designated as LXRα (NR1H₃) and LXRβ (NR1H2, Cell 97, 161-163, 1999). The compounds of Formula I are LXR ligands and individually may vary in their selectivity for one or the other of LXRα and LXRβ, or they may have mixed binding affinity for both LXRα and LXRβ. More particularly, the tested compounds included within the scope of this invention have an IC₅₀ less than or equal to 2 μM for at least one of either the LXRα or LXRβ receptors employing the LXR radioligand competition scintillation proximity assays described below in the Example section. Preferred tested compounds of Formula I bind to the human LXRα receptor and have an IC₅₀ less than or equal to 300 μM for the LXRα receptor.

Compound A is used in the following assays and has the following structural formula: 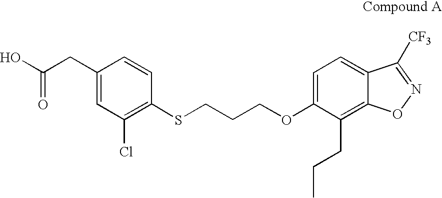
Compound A and related compounds are disclosed along with methods for making them in WO97/28137.

The compounds in the following examples were characterized using ¹H NMR at 400 or 500 MHz field strength, and/or by ESI mass spectroscopy (MS).

### EXAMPLE 1

Radioligand Competition Binding Scintillation Proximity Assays

Preparation of Recombinant Human LXRα and LXRβ

Human LXRα and LXRβ were expressed as GST-fusion proteins in E. coli. The ligand binding domain cDNAs for human LXRα (amino acids 164-447) and human LXRβ (amino acids 149-455) were subcloned into the pGEX-KT expression vector (Pharmacia). E. coli containing the respective plasmids were propagated, induced, and harvested by centrifugation. The re-suspended pellet was broken in a French press and debris was removed by centrifugation. Recombinant human LXR receptors were purified by affinity chromatography on glutathione sepharose and receptor was eluted with glutathione. Glycerol was added to a final concentration of 50% to stabilize the receptor and aliquots were stored at −80° C.

Binding to LXRα:

For each assay, an aliquot of human GST-LXRα receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/mi yttrium silicate protein A coated SPA beads (Amersham Pharmacia Biotech, Inc.), 8.3 μg/ml anti-GST antibody (Amersham Pharmacia Biotech, Inc.), 0.1% non-fat dry milk and 25 nM [³H₂]Compound A (13.4 Ci/mmole), ±test compound. After incubation for ˜16 h at 15° C with shaking, the assay plates were counted in a Packard Topcount. In this assay the K_{d} for Compound A for LXRα is ≈15 nM.

Binding to LXRβ:

For each assay, an aliquot of human GST-LXRβ ligand binding domain receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.) 0.1% non-fat dry milk and 25 nM [³H₂]Compound A (13.4 Ci/mmole), ±test compound. After incubation for −16 h at 15° C with shaking, the assay plates were counted in a Packard Topcount. In this assay the K_{d} for Compound A for LXRβ is ˜10 nM.

Results:

Representative tested compounds of Formula I are ligands for human LXRα and/or human LXRβ, each having an IC₅₀≦1,800 nM for at least one of the LXRα receptor or the LXRβ receptor, and preferred tested compounds having an IC₅₀ of 300 nM or less for at least one of the LXRα receptor or the LXRβ receptor.

### EXAMPLE 2

Transactivation Assay

Plasmids

Expression constructs were prepared by inserting the ligand binding domain (LBD) of human LXRα and LXRβ cDNAs adjacent to the yeast GAL4 transcription factor DNA binding domain (DBD) in the mammalian expression vector pcDNA3 to create pcDNA3-LXRα/GAL4 and pcDNA3-LXRβ/GAL4, respectively. The GAL4-responsive reporter construct, pUAS(5X)-tk-luc, contained 5 copies of the GAL4 response element placed adjacent to the thymidine kinase minimal promoter and the luciferase reporter gene. The transfection control vector, pEGFP-N1, contained the Green Fluorescence Protein (GFP) gene under the regulation of the cytomegalovirus promoter.

Assay

HEK-293 cells were seeded at 40,000 cells/well in 96 well plates in Dulbecco's modified Eagle medium (high glucose) containing 10% charcoal stripped fetal calf serum, 100 units/ml Penicillin G and 100 μg/ml Streptomycin sulfate at 37° C. in a humidified atmosphere of 5% CO₂. After 24 h, transfections were performed with Lipofectamine (Gibco-BRL, Gaithersburg, Md.) according to the instructions of the manufacturer. In general, transfection mixes contained 0.002 μg of LXRα/GAL4 or LXRβ/GAL4 chimeric expression vectors, 0.02 μg of reporter vector pUAS(5X)-tk-luc and 0.034 μg of pEGFP-N1 vector as an internal control of transfection efficiency. Compounds were characterized by incubation with transfected cells for 48 h across a range of concentrations. Cell lysates were prepared from washed cells using Cell Lysis Buffer (Promega) according to the manufacturer's directions. Luciferase activity in cell extracts was determined using Luciferase Assay Buffer (Promega) in a ML3000 luminometer (Dynatech Laboratories). GFP expression was determined using the Tecan Spectrofluor Plus at excitation wavelength of 485 nm and emission at 535 nm. Luciferase activity was normalized to GFP expression to account for any variation in efficiency of transfection.

Results with representative tested compounds of Formula I for LXRα transactivation having an EC₅₀ of ≦5,500 nM for at least one of the LXRα receptor or the LXRβ receptor, and preferred tested compounds having an EC50 of 1,000 nM or less for at least one of the LXRα receptor or the LXRβ receptor.

### EXAMPLE 3

To assess the relevant biological activity of the LXR agonists, certain compounds were tested for their ability to increase cholesterol efflux from cultured human cells, as described by Sparrow et al., JBC, 277, 10021-10027, Mar. 22, 2002. Caco-2 cells, which are of human origin, were obtained from ATCC and grown in Opti-MEM (Gibco #51985-034) containing 10% FCS, non-essential amino acids (Gibco #11140-050), and vitamins (Gibco # 11120-052). Caco-2 cells were plated at 100,000 cells/well in 48-well plates. After four days the cells had reached confluence, and were then labeled with ³H-cholesterol by incubation for 24 hours in fresh growth media containing ³H-cholesterol (10 μCi/ml). Following labeling with ³H-cholesterol, cells were washed and incubated an additional 24 hours in serum-free media containing 1 mg/ml BSA, to allow for equilibration of ³H-cholesterol with intracellular cholesterol. Cholesterol efflux was initiated by adding 10 μg/ml apoA-I, with or without compound, in serum-free medium. Compounds were added to cell culture medium from DMSO solutions, and control cells received an equivalent amount of DMSO, never exceeding 0.1%. After 24 hours, media were harvested and cells dissolved in 0.1 M NaOH. Media were briefly centrifuged to remove non-adherent cells, and then aliquots of both the supernatants and the dissolved cells were subjected to liquid scintillation spectrometry to determine radioactivity. Cholesterol efflux is expressed as a percentage, calculated as (³H-cholesterol in medium/(³H-cholesterol in medium +³H-cholesterol in cells))×100.

Table 1 shows cholesterol efflux results for the compound made in Example 25. Results are given as mean of quadruplicate incubations.

TABLE 1

| Compound concentration | |
|---|---|
| (nM) | % cholesterol efflux |
| 10 | 3.4 |
| 30 | 4.4 |
| 100 | 4.3 |
| 300 | 6.6 |
| 1000 | 7.4 |
| 3000 | 7.7 |

Table 2 shows cholseterol efflus results for the cmpound made in Example 40. Results are given as mean of quadruplicate incubtions.

TABLE 2

| Compound concentration | |
|---|---|
| (nM) | % cholesterol efflux |
| 0 | 3.8 |
| 10 | 4.9 |
| 30 | 5.2 |
| 100 | 5.9 |
| 300 | 6.3 |
| 1000 | 6.5 |
| 3000 | 7.7 |

### EXAMPLE 4

### Preparation of 2,4-dihydroxy-3-propyl-1′,1′,1′-trifluoroacetophenone

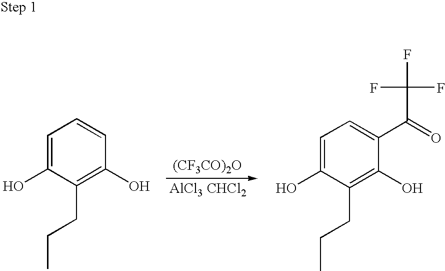 A solution of 2-propylresorcinol (5.0 grams) and trifluoroacetic anhydride (9.6 mL) in 1,2-dichloroethane (30.0 mL) was treated with aluminum chloride (4.38 grams). This mixture was stirred overnight. The reaction mixture was partitioned between methylene chloride and water. The organic phase was dried over sodium sulfate and filtered. The solvent was evaporated and the resulting solid was recrystallized from methylene chloride and cyclohexane (1:1) to give the titled compound.
Selected Signals: ¹H NMR (CDCl₃) δ7.59 (d, 1H), 6.24 (d, 1H), 5.92 (s, 1H), 2.63 (t, 2H), 1.74 (s, 1H), 1.58 (m, 2H), 0.98 (t, 3H).

### Preparation of 3-Trifluoromethyl-7-propyl-6-hydroxybenzisoxazole

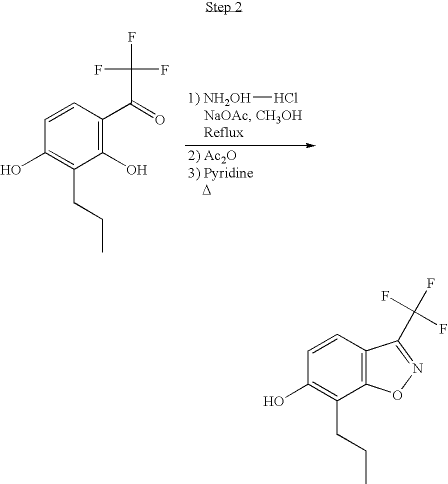 A mixture of 2,4-dihydroxy-3-propyl-1′,1′,1′-trifluoroacetophenone (2.5 grams), sodium acetate (4.18 grams), hydroxylamine hydrochloride (3.59 grams) and methanol (80 mL) was heated under reflux overnight. The solvent was then evaporated and the resulting solid was partitioned between ethyl acetate and pH 7 buffer. The organic phase was separated and washed with brine. The organic phase was dried over sodium sulfate and the solvent was evaporated to give an oil. The oil was then dissolved in acetic anhydride. The solution was stirred for two hours, then the acetic anhydride was evaporated in vac. The residue was partitioned between ethyl acetate and pH 7 buffer and the organic phase was dried over sodium sulfate. The organic phase was evaporated to give an oil. The oil was dissolved in pyridine and refluxed overnight. The solvent was evaporated in vacuo to give an oil which was chromatographed on silica gel using ethyl acetate and hexanes (1:4) to give the titled compound.
Selected Signals: ¹H NMR (CDCl₃) δ7.46 (d, 1H), 6.92 (d, 1H), 5.42 (bs, 1H), 2.89 (t, 2H), 1.74 (m, 2H), 0.98 (t, 3H).

### EXAMPLE 5

### Preparation of 7-propyl-3-(trifluoromethyl)-6-(3-bromopropyloxy)-1,2-benzisoxazole

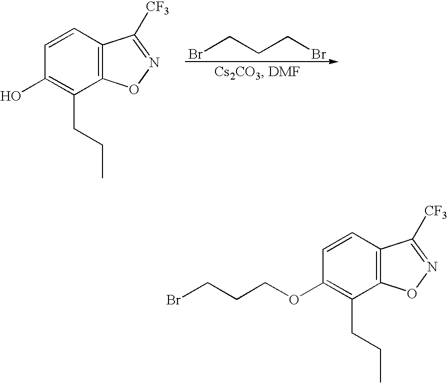 To a DMF solution (50 mL) of 6-hydroxy-7-propyl-3-(trifluoromethyl)-1,2-benzisoxazole (prepared in Example 4 step 2, 5 grams, 20.4 mmol) was added 1,3-dibromopropane (10 mL, 98.5 mmol), followed by cesium carbonate (10 grams, 30.7 mmol). The mixture was stirred at room temperature overnight. After aqueous/ether work-up and silica gel chromatography (hexanes: 2.5% ethyl acetate), the titled compound was obtained.
Selected Signals: ¹H NMR (CDCl₃); δ7.59 (d, 2H, J=8.8 Hz), 7.10 (d, 2H, J=8.8 Hz), 4.27 (t, 2H, J=5.8 Hz), 3.66 (t, 2H, J=6.4 Hz), 2.93 (t, 2H, J=7.5 Hz), 2.41 (pent, 2H, J=6.0 Hz), 1.72 (sext, 2H, J=7.5 Hz), 0.99 (t, 3H, J=7.5 Hz).

### EXAMPLE 6

### Preparation of 7-propyl-3-(trifluoromethyl)-6-(3-bromopropyloxy)-1,2-benzisoxazole

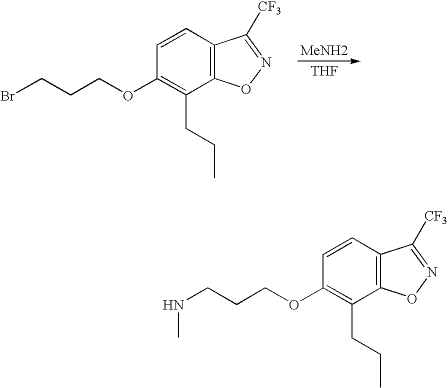

To a THF solution (50 mL) of 7-propyl-3-(trifluoromethyl)-6-(3-bromopropyloxy)-1,2-benzisoxazole (prepared in Example 5, 1.0 grams, 2.73 mmol) was added methylamine (15 mL, 27.3 mmol) and the mixture was stirred at room temperature overnight. After aqueous/ether work-up and silica gel chromatography methanol:methylene chloride (10/90) 2% NH₄OH, the titled compound was obtained.

Selected Signals: ¹H NMR (CDCl₃); δ7.59 (d, 2H, J=8.8 Hz), 7.10 (d, 2H, J=8.8 Hz), 4.27 (t, 2H, J=5.8 Hz), 3.66 (t, 2H, J=6.4 Hz), 2.93 (t, 2H, J=7.5 Hz), 2.41 (pent, 2H, J=6.0 Hz), 1.72 (sext, 2H, J=7.5 Hz), 0.99 (t, 3H, J=7.5 Hz).

### EXAMPLE 7

### Preparation of 1-(3-{[7-propyl-3-(trifluoromethyl)-1,2-benzisoxazol-6-yl]oxy}propyl)-1H-indole-5-carboxylic acid

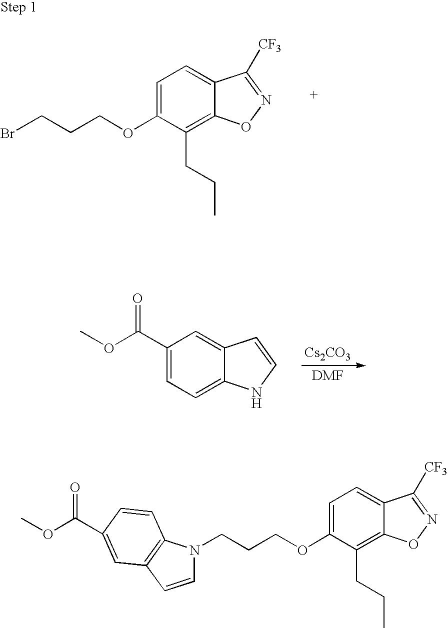 To a solution of the bromide (prepared according to the procedure of Example 5, 0.74 g, 2.02 mmol) and methyl indole-5-carboxylate (0.118 g, 0.674 mmol) in DMF (10 mL) was added Cs₂CO₃ (0.242 g, 0.72 mmol). The reaction mixture was stirred at room temperature overnight. H₂O was then added to the reaction mixture and the mixture extracted with ether (3×80 mL). The combined ether extracts were washed with brine and dried over Na₂SO₄. Na₂SO₄ was then filtered off and solvent was evaporated in vac. The residue was chromatographed on silica gel using ethyl acetate:hexanes (1:4) to give the titled compound. 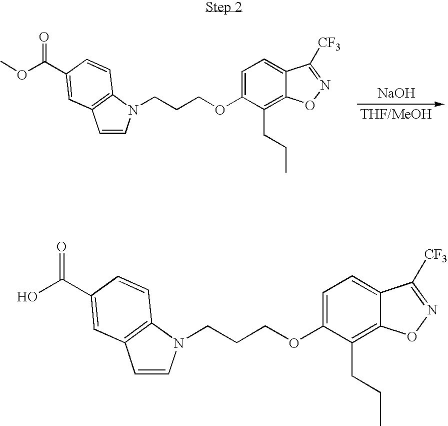
To a solution of the ester (prepared according to the procedure of Example 7 Step 1, 0.232 g, 0.504 mmol) in THF (2 mL) and MeOH (8 mL) was added NaOH (1N, 2 mL). The reaction mixture was stirred at room temperature overnight. The volume was reduced in vac, the residue was neutralized with 1 N HCL and extracted with ethyl acetate. The combined ethyl acetate extracts were washed with brine and dried over Na₂SO₄. Na₂SO₄ was filtered off and solvent was evaporated in vac and the residue was chromatographed on silica gel using ethyl acetate:hexanes (20:80) 2.5% acetic acid to give the titled compound.
¹H NMR (CDCl₃) δ8.47 (m, 1H), 7.94 (d, 1H, J=9.0 Hz), 7.54 (d, 1H, J=9.0), 7.39 (d, 1H, J=8.5), 7.14 (m, 1H), 6.94 (d, 1H, J=5.0), 6.63 (m, 1H), 4.45 (t, 2H, J=6.5), 4.03 (t, 2H, J=5.5), 3.00 (t, 2H, J=7.5), 2.40 (m, 2H), 1.80 (m, 5H), 1.04 (t, 3H, J=7.5)
MS: m/z=447 (M+H).

### EXAMPLE 8

### Preparation of 4-[methyl(3-{[7-propyl-3-(trifluoromethyl)-1,2-benzisoxazol-6-yl]oxy}propyl)amino]benzoic acid

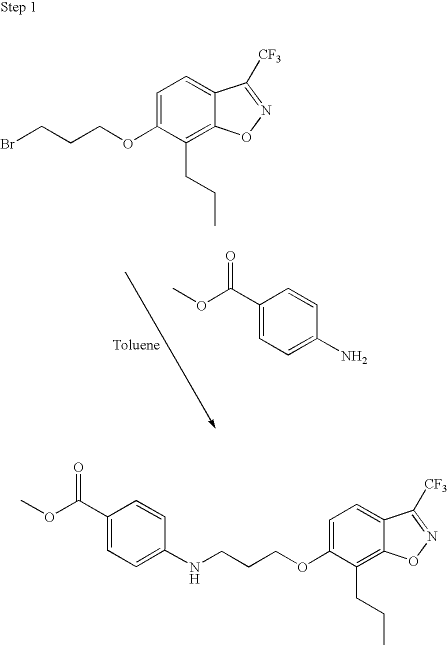 The indicated aniline (0.44 g, 2.91 mmol) was added to a solution of the bromide (prepared according to the procedure of Example 5, 0.21 g, 57 mmol) in Toluene (5 mL). The reaction mixture was heated at reflux overnight. The reaction mixture was cooled to room temperature, H₂O was added and the mixture extracted with ethyl acetate (3×50 mL). The combined ethyl acetate extracts were washed with brine and dried over Na₂SO₄. Na₂SO₄ was then filtered off and solvent was evaporated in vac and the residue was chromatographed on silica gel using acetone:hexanes (1:4) to give the titled compound. 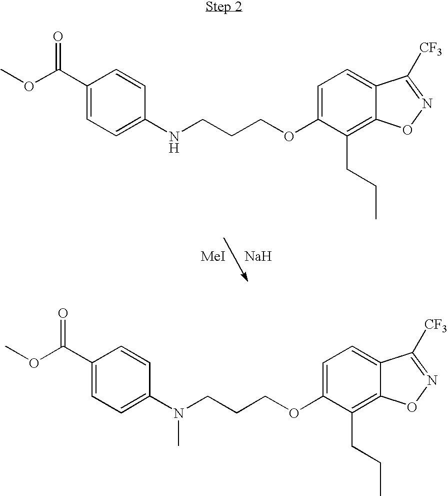
The indicated aniline (prepared according to the procedure of Example 8 Step 1, 0.12 g, 0.27 mmol) and NaH (32.4 mg, 0.81 mmol) were combined in THF (4 mL) at −20 ° C. and stirred under N₂ for 10 minutes. MeI (81 μL, 1.35 mmol) was added to the reaction mixture and stirred at room temperature overnight. The mixture was partitioned between ethyl acetate and H₂O and the organic phase was dried over sodium sulfate and filtered. The solvent was evaporated in vac to give the titled compound. 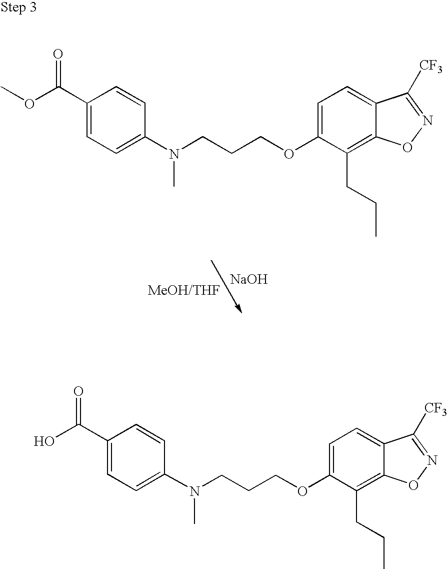
NaOH aq (1N, 0.5 mL) was added to the aniline (prepared according to the procedure of Example 8 Step 2, 50.0 mg, 0.10 mmol) in MeOH (1 mL) and THF (1 mL) at RT. The mixture was stirred at room temperature overnight. The reaction mixture was diluted with ethyl acetate and neutralized with acetic acid. Then the solvent was evaporated in vac and chromatographed on silica gel using acetic acid ,ethyl acetate and hexanes (2.5:30:70) to give the titled compound.
¹H NMR (CDCl₃) δ7.96 (d, 2H, J=9.0 Hz), 7.59 (d, 1H, J=9.0), 7.06 (d, 1H, J=9.0), 6.76 (d, 2H, J=8.5), 4.18 (t, 1H, J=5.5), 3.72 (t, 1H, J=7.0), 3.10 (s, 3H), 3.00 (t, 2H, J=7.5), 2.20 (m, 2H), 1.78 (m, 2H), 1.04 (t, 3H, J=7.0).
MS: m/z=436 (M+H).

### EXAMPLE 9

### Preparation of 3-methyl-4-[methyl(3-{[7-propyl-3-(trifluoromethyl)-1,2-benzisoxazol-6-yl]oxy}propyl)amino]benzoic acid

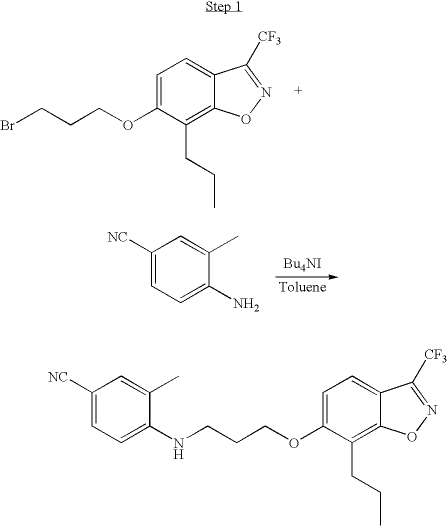 To a solution of the bromide (prepared according to the procedure of Example 5, 0.134 g, 0.366 mmol) in toluene (3 mL) was added the indicated aniline (0.145 g, 1.09 mmol) and Bu₄NI (1.35 g, 0.366 mmol). The reaction mixture was heated at reflux overnight. The reaction mixture was cooled to room temperature and H₂O was added. The mixture was extracted with ethyl acetate (3×80 mL). The combined ethyl acetate extracts were washed with brine and dried over Na₂SO₄. Na₂SO₄ was then filtered off and solvent was evaporated in vac and the residue was chromatographed on silica gel using acetone:hexanes (25:75) 1% Et₃N to give the titled compound. 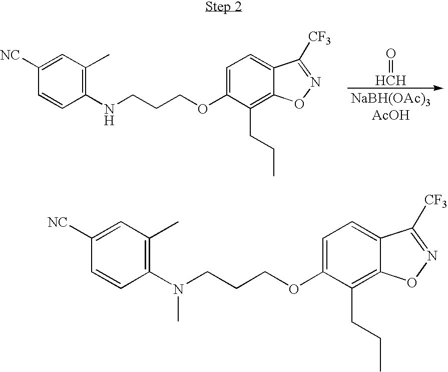
NaBH(OAc)₃ (0.132 g, 0.6 mmol) was added to the aniline (prepared according to the procedure of Example 9 Step 1, 88.2 mg, 0.211 mmol), formaldehyde (0.17 ml, 2.11 mmol) and acetic acid (0.076 mL, 1.27 mmol) in dichloroethane (2 mL). The reaction mixture was stirred at room temperature overnight. The reaction mixture was diluted with H₂O and extracted with ethyl acetate (3×80 mL). The combined ethyl acetate extracts were washed with brine and dried over Na₂SO₄. Na₂SO₄ was then filtered off and solvent was evaporated in vac and the residue was chromatographed on silica gel using acetone:hexanes (25:75) 1% Et₃N to give the titled compound. 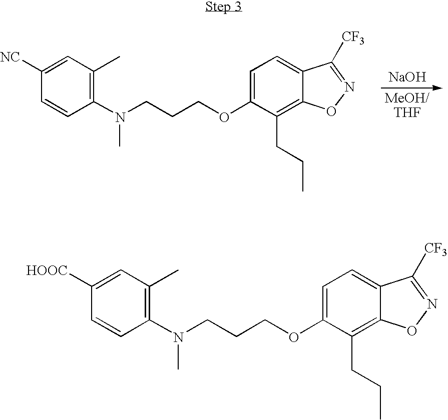
NaOH (aqueous 50%, 1 mL) was added to the nitrile (prepared according to the procedure of Example 9 Step 2, 74.0 mg, 0.17 mmol) in MeOH (4 mL) and THF (2 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (20:80) 2.5% acetic acid to give the titled compound.
¹H NMR (CDCl₃) δ7.89 (m, 2H), 7.57 (d, 1H, J 9.0 Hz), 7.08 (d, 1H, J=9.5), 7.02 (d, 1H, J=9.0), 4.16 (t, 2H, J=6.0), 3.30 (t, 2H, J=7.0), 2.91 (t, 2H, J=7.5), 2.84 (s, 3H), ), 2.36 (s, 3H), 2.14 (m, 2H), 1.70 (m, 2H), 0.97 (t, 3H, J=7.5).
MS: m/z=451 (M+H).

### EXAMPLE 10

### Preparation of [2-oxo-1-(3-{[7-propyl-3-(trifluoromethyl)-1.2-benzisoxazol-6-yl]oxy}propyl)-1,2,3,4-tetrahydroquinolin-6-yl]acetic acid.

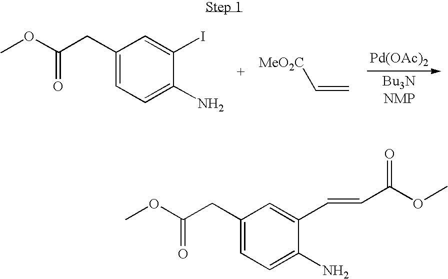 Methyl acrylate (0.186 mL, 0.214 mmol) was added to a solution of the indicated iodide (0.3 g, 1.03 mmol), Pd(OAc)₂ (24 mg, 0.107 mmol) and Bu₃N (0.246 mL, 0.107 mmol) in 1-methyl-2-pyrrolidine (5 mL). The mixture was heated to 80° C. for 2 hours. The reaction mixture was cooled to room temperature and quenched with water, followed by extraction with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (30:70) to give the titled compound.

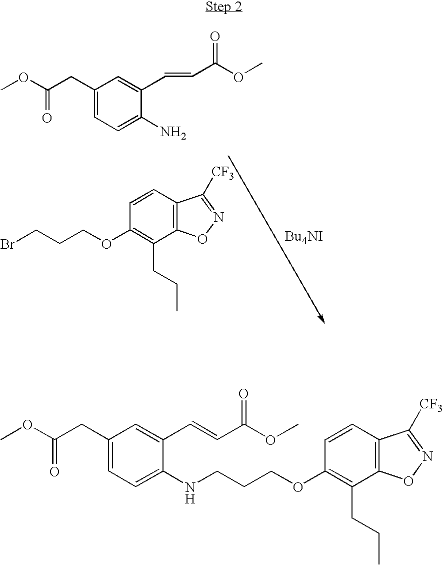

A mixture of the aniline (prepared according to the procedure of Example 10 Step 1, 60.0 mg, 0.20 mmol), the indicated bromide (prepared according to Example 5, 73.5 mg, 0.20 mmol) and Bu₄NI (0.178 g, 0.40 mmol) in toluene (3 mL) was heated at reflux under N₂ overnight. The solvent was evaporated in vac and the residue was chromatographed on silica gel using ethyl acetate and hexanes (3:7) to give the titled compound. 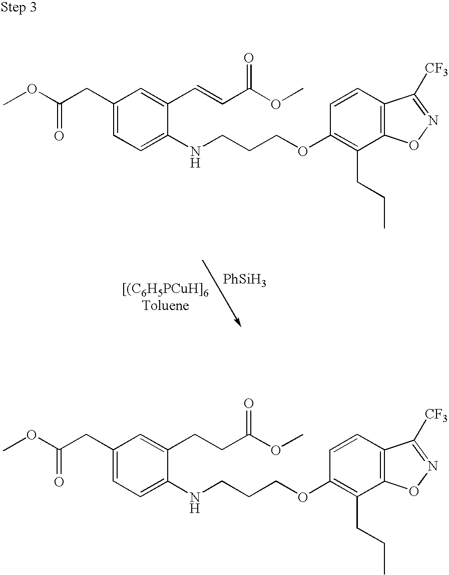
A mixture of the aniline (prepared according to the procedure of Example 10 Step 2, 11.0 mg, 0.0205 mmol), PhSiH₃ (4.0 μL, 0.0307 mmol) and [(C₆H₅)₃PCuH]₆ (2.11 mg, 0.001 mmol) in toluene (2 mL) was stirred at room temperature under N₂ overnight. The solvent was evaporated in vac and the residue was chromatographed on silica gel using ethyl acetate and hexanes (4:6) to give the titled compound. 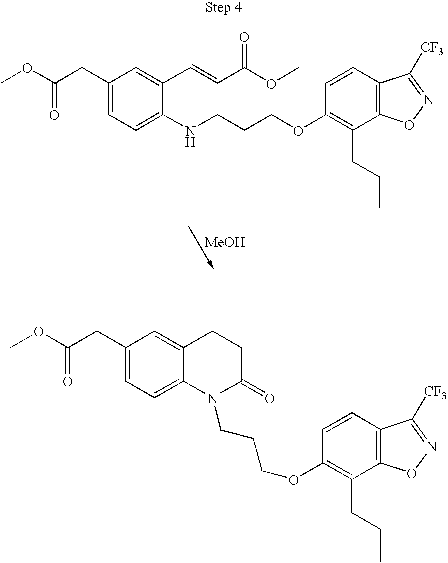
A solution of the aniline (prepared according to the procedure of Example 10 Step 3, 4.0 mg, 0.0075 mmol) in MeOH (2 mL) was heated to 60° C. overnight. The solvent was evaporated in vac and the residue chromatographed on silica gel using ethyl acetate and hexanes (4:6) to give the titled compound. 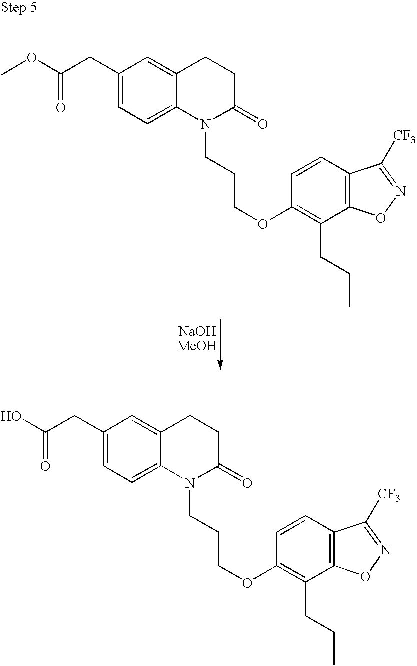
NaOH aq (16 μL) was added to the aniline (prepared according to the procedure of Example 10 Step 4, 4.0 mg, 0.008 mmol) in MeOH (0.5 mL) and THF (0.3 mL). The mixture was stirred at room temperature overnight. The reaction mixture was diluted with ethyl acetate and neutralized with acetic acid. The solvent was evaporated in vac and the residue chromatographed on silica gel using ethyl acetate and hexanes (40:60) 2.5% acetic acid, to give the titled compound.
¹H NMR (CDCl₃) δ7.59 (d, 1H, J=9.0 Hz), 7.17 -7.06 (m, 4H), 4.20 (m, 4H), 3.68 (s, 2H), 2.99 (t, 2H, J=8.0), 2.94 (m, 2H), 2.69 (m, 2H), 2.26 (m, 2H), 1.70 (m, 2H), 1.01 (t, 1H, J=7.5).
MS: m/z =491.5 (M+H).

### EXAMPLE 11

### Preparation of 1-(3-{[7-propyl-3-(trifluoromethyl)-1,2-benzisoxazol-6-yl]oxy}propyl)-1,2,3,4-tetrahydroquinoline-6-carboxylic acid

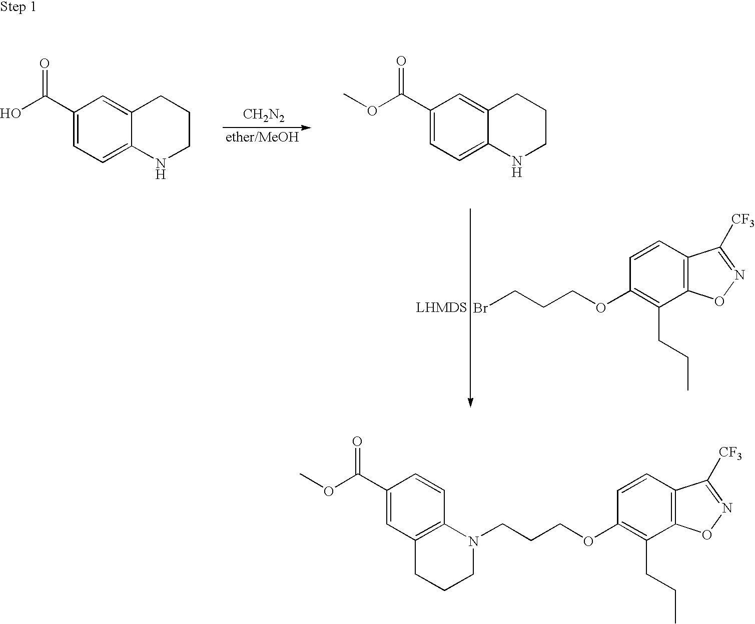 Excess CH₂N₂ in ether was added to a solution of tetrahydroquinoline carboxylic acid (0.8 g, 4.51 mmol) in ether (30 mL) and MeOH (3 mL) and the mixture stirred at room temperature for 2 hours. Excess diazomethane was destroyed with MgSO₄ and the solvent was evaporated in vac. The ester was dissolved in THF (6 mL) and cooled to 0° C., followed by addition of LiHMDS (1 M hexanes, 5.42 mL, 5.42 mmol) and stirring for 5 minutes. The indicated bromide (prepared as in Example 5, 1.81 g, 5.0 mmol) was added and the mixture was allowed to stir at 0° C. overnight. The reaction mixture was quenched with NH₄Cl (sat'd aq) at 0° C. and the resulting mixture extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (1:4) to give titled compound. 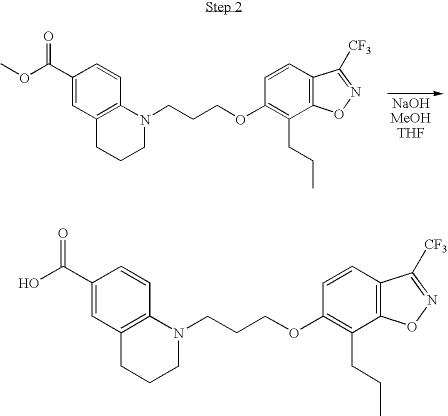
NaOH aq (1 M, 0.35 mL, 0.35 nrnol) was added to the methyl ester (prepared according to the procedure of Example 11 Step 1, 41 mg, 0.09 mmol) in MeOH (0.6 mL) and THF (0.15 mL). The mixture was stirred at room temperature overnight. The reaction mixture was diluted with ethyl acetate and neutralized with acetic acid. The solvent was evaporated in vac and the residue was chromatographed on silica gel using ethyl acetate and hexanes (20:80) 2.5% acetic acid to give the titled compound.
¹H NMR (CDCl₃) δ7.79 (d, 1H, J=8.5 Hz), 7.71 (s, 1H), 7.57 (d, 1H, J=9.0), 7.07 (d, 1H, J=8.5), 6.96 (d, 1H, J=8.5), 4.19 (t, 2H, J=5.5), 3.64 (t, 2H, J=7.0), 3.31 (t, 2H, J=5.5), 2.99 (t, 2H, J=7.5), 2.78 (t, 2H, J=6.5), 2.17 (m, 2H), 1.98 (m, 2H), 1.78 (m, 2H), 1.03 (t, 3H, J=7.0).
MS: m/z=463 (M+H).

### EXAMPLE 12

### Preparation of [1-(3-{[7-propyl-3-(trifluoromethyl)-1,2-benzisoxazol-6-yl]oxy}propyl)-1,2,3,4-tetrahydroquinolin-6-yl]acetic acid.

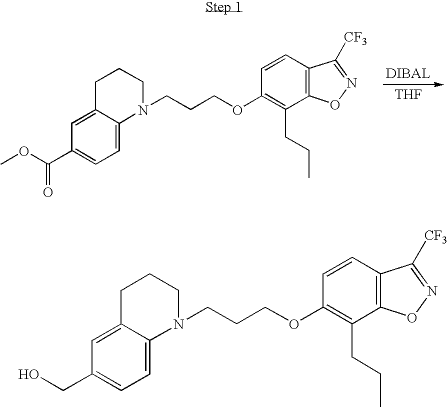 A solution of the tetrahyro-6-quinoline carboxylic methyl ester (prepared according to the procedure of Example 11 Step 1, 0.18 g, 0.38 mmol) in THF (5 mL) was cooled to −20° C., followed by addition of DIBAL (1 M Hexanes, 1.89 mL, 1.89 mmol). The reaction mixture was stirred under N₂ for ˜2 hours. NaOH aq (1N, 20 mL) was added into reaction mixture and the resulting mixture was extracted with ethyl acetate. The combined ethyl acetate extracts were washed with brine and dried over Na₂SO₄. Na₂SO₄ was then filtered off and solvent was evaporated in vac to give the titled compound. 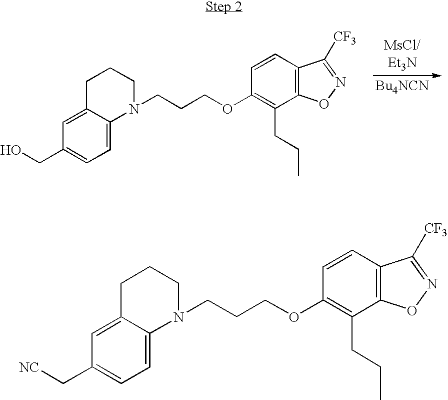
A mixture of the tetrahydroquinoline (prepared according to the procedure of Example 12 Step 1, 155.8 mg, 0.35 mmol), Et₃N (0.15 mL, 1.04 mmol) and MsCl (32.3 μL, 0.420 mmol) in THF (3 mL) was stirred at −30° C. under N₂ for 10 minutes. Bu₄NCN (0.59 g, 2.09 mmol) was added to the reaction mixture, the acetone-dry ice bath was removed and the reaction was stirred vigorously for 3 hours. The reaction mixture was quenched with NaOH aq (1N, 30 mL) and the resulting mixture partitioned between methylene chloride and water. The combined methylene chloride extracts were dried over sodium sulfate and filtered. The solvent was evaporated in vac and the residue was chromatographed on silica gel using ethyl acetate and hexanes (2:8) to give the titled compound. 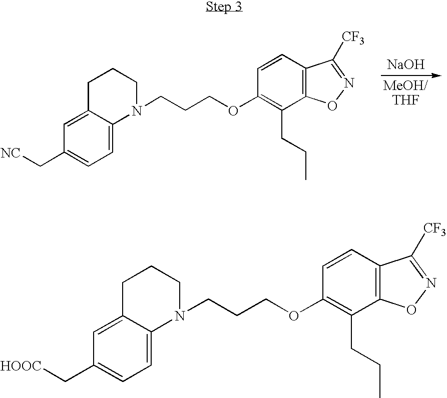
Aqueous NaOH (50%, 1 mL) was added to the nitrile (prepared according to the procedure of Example 12 Step 2,45.0 mg, 0.098 mmol) in MeOH (4 mL) and THF (2 mL). The reaction mixture was heated at reflux overnight. The reaction mixture was cooled to room temperature, neutralized with acetic acid and extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes: (20:80) 2.5% acetic acid to give the titled compound.
¹H NMR (CDCl₃) δ7.57 (d, 1H, J=9.0 Hz), 7.07 (d, 1H, J=8.5), 6.96 (d, 1H, J=8.5), 6.91 (s, 1H), 6.61 (d, 1H, J=8.0), 4.19 (t, 2H, J=5.5), 3.54 (m, 4H), 3.31 (t, 2H, J=5.5), 2.99 (t, 2H, J=7.5), 2.78 (t, 2H, J=6.5), 2.17 (m, 2H), 1.98 (m, 2H), 1.78 (m, 2H), 1.03 (t, 3H, J=7.0).
MS: m/z=477 (M+H).

### EXAMPLE 13

### Preparation of 3-chloro-4-[methyl(3-{[7-propyl-3-(trifluoromethyl)-1,2-benzisoxazol-6-yl]oxy}propyl)amino]benzoic acid.

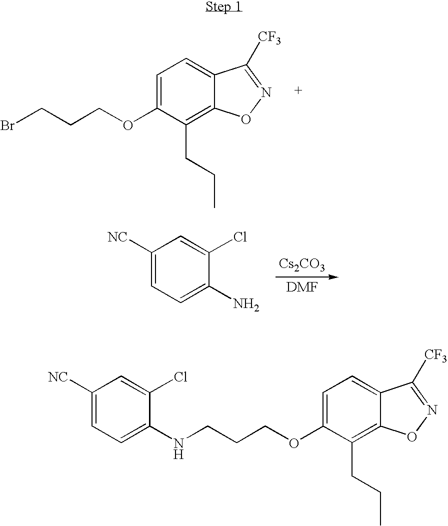 The indicated aniline (0.22 g, 1.09 mmol) was added to a solution of the bromide (prepared according to the procedure of Example 5, 0.179 g, 0.49 mmol) in DMF (5 miL) followed by Cs₂CO₃ (0.18 g, 0.53 mmol). The reaction mixture was heated to 80° C. overnight. The reaction mixture was partitioned between ethyl acetate and water. The combined ethyl acetate extracts were dried over sodium sulfate and filtered. The solvent was evaporated and the resulting solid was chromatographed on silica gel using ethyl acetate and hexanes (20:80) 1% Et₃N to give the titled compound. 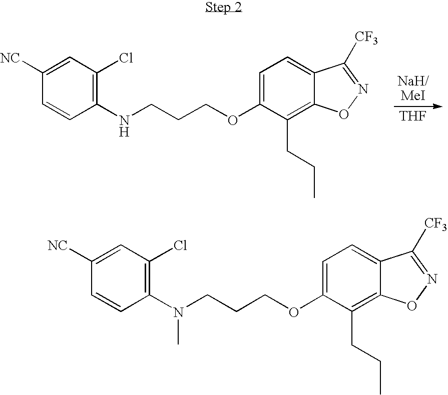
NaH (16.3 mg, 0.41 mmol) was added to the aniline (prepared according to the procedure of Example 13 Step 1, 59.2 mg, 0.14 mmol) in THF (3 mL) at −20° C. The mixture was stirred under N₂ for 10 minutes. MeI (42 μL, 0.68 mmol) was added to the reaction mixture and the resulting mixture was stirred at room temperature overnight. The solvent was evaporated in vac and the residue was chromatographed on silica gel using ethyl acetate:hexanes (2:8) to give the titled compound. 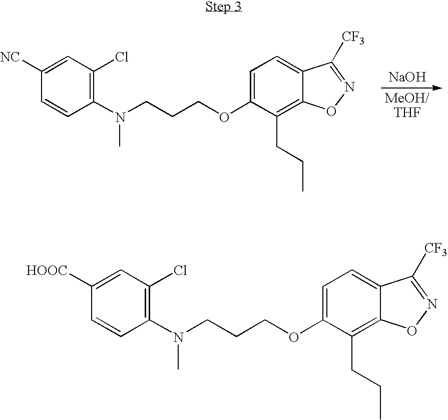
Aqueous NaOH (50%, 0.5 mL) was added to the nitrile (prepared according to the procedure of Example 13 Step 2, 32.0 mg, 0.07 mmol) in MeOH (1 mL) and THF (0.5 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (20:80) 2.5% acetic acid to give the titled compound.
¹H NMR (CDCl₃) δ8.09 (s, 1H), 7.95 (d, 1H, J=8.5 Hz), 7.57 (d, 1H, J=8.5), 7.12 (d, 1H, J=8.5), 7.05 (d, 1H, J=9.05), 4.19 (t, 2H, J=6.0), 3.48 (t, 2H, J=7.0), 2.97 (s, 3H), 2.93 (t, 2H, J=7.5), 2.22 (m, 2H), 1.71 (m, 2H), 0.98 (t, 3H, J=7.5).
MS: m/z=471.5 (M+H).

### EXAMPLE 14

### Preparation of 2-chloro-4-[methyl(3-{[7-propyl-3-(trifluoromethyl)-1,2-benzisoxazol-6-yl]oxy}propyl)amino]benzoic acid

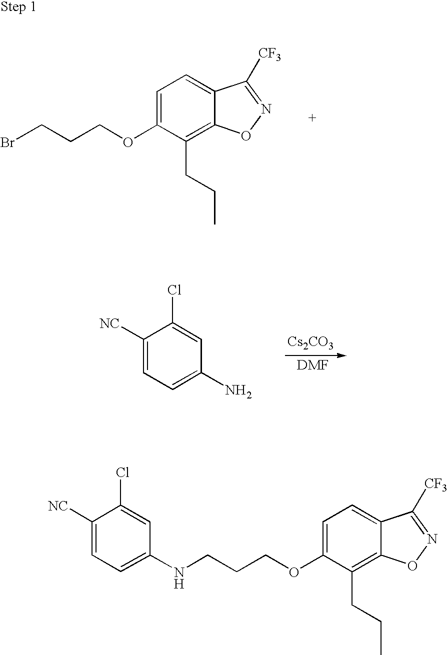 The indicated aniline (0.225 g, 1.46 mmol) was added to a solution of the bromide (prepared according to the procedure of Example 5, 0.178 g, 0.49 mmol) in DMF (5 mL) followed by Cs₂CO₃ (0.18 g, 0.53 mmol). The reaction mixture was heated to 80° C. overnight. The reaction mixture was partitioned between ethyl acetate and water. The combined ethyl acetate extracts were dried over sodium sulfate and filtered. The solvent was evaporated and the resulting solid was chromatographed on silica gel using ethyl acetate and hexanes (20:80) 1% Et₃N to give the titled compound. 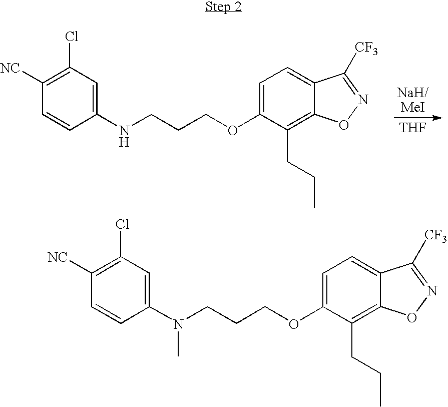
NaH (50.7 mg, 0.27 mmol) was added to the aniline (prepared according to the procedure of Example 14 Step 1, 184.9 mg, 0.423 mmol) in THF (3 mL) at −20° C. The mixture was stirred under N₂ for 10 minutes. MeI (0.132 mL, 2.1 mmol) was added to the reaction mixture and the resulting mixture stirred at RT overnight. The solvent was evaporated in vac and the residue was chromatographed on silica gel using ethyl acetate:hexanes (3:7) to give the titled compound. 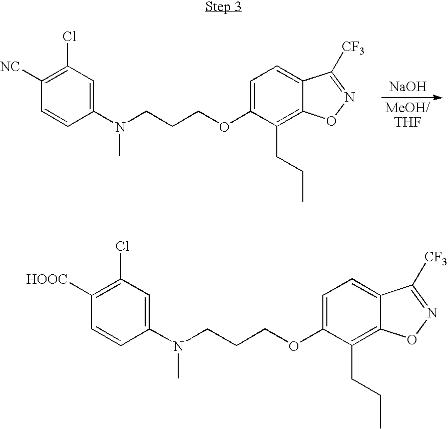
Aqueous NaOH (50%, 2.0 mL) was added to the nitrile (prepared according to the procedure of Example 14 Step 2, 0.21 g, 0.46 mmol) in MeOH (5 mL) and THF (1.5 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac and the residue was chromatographed on silica gel using ethyl acetate:hexanes (30:70) 2.5% acetic acid to give the titled compound.
¹H NMR (CDCl₃) δ7.97 (d, 1H, J=8.5 Hz), 7.57 (d, 1H, J=9.0), 7.06 (d, 1H, J=8.5), 6.75 (m, 1H), 6.64 (m, 1H), 4.19 (t, 2H, J=6.0), 3.48 (t, 2H, J=7.0), 2.97 (s, 3H), 2.93 (t, 2H, J=7.5), 2.22 (m, 2H), 1.71 (m, 2H), 0.98 (t, 3H, J=7.5).
MS: m/z=471.5 (M+H).

### EXAMPLE 15

### Preparation of 4-[methyl(3-{[7-propyl-3-(trifluoromethyl)-1,2-benzisoxazol-6-yl]oxy}propyl)amino]-3-(trifluoromethoxy)benzoic acid

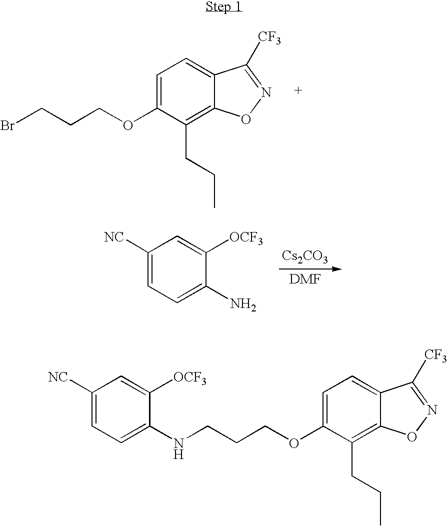 The indicated aniline (0.1 g, 1.46 mmol) was added to a solution of the bromide (prepared according to the procedure of Example 5, 0.178 g, 0.49 mmol) and Cs₂CO₃ (0.18 g, 0.54 mmol) in DMF (5 mL). The resulting mixture was heated at 80° C. overnight. The reaction mixture was partitioned between ethyl acetate and water. The combined ethyl acetate extracts were dried over sodium sulfate and filtered. The solvent was evaporated and the resulting solid was chromatographed on silica gel using ethyl acetate and hexanes (20:80) 1% Et₃N to give the compound pictured above. 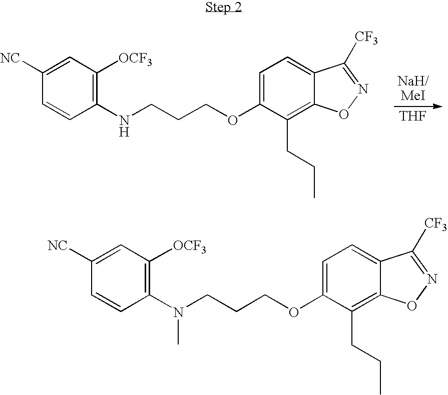
NaH (74.2 mg, 1.86 mmol) was added to the aniline (prepared according to the procedure of Example 15 Step 1, 150.5 mg, 0.31 mmol) in THF (3 mL) at −20° C. and stirred under N₂ for 10 minutes. MeI (0.231 mL, 3.71 mmol) was added to the reaction mixture and the resulting mixture stirred at room temperature overnight. The solvent was evaporated in vac and the residue was chromatographed on silica gel using ethyl acetate:hexanes (3:7) to give the compound pictured above. 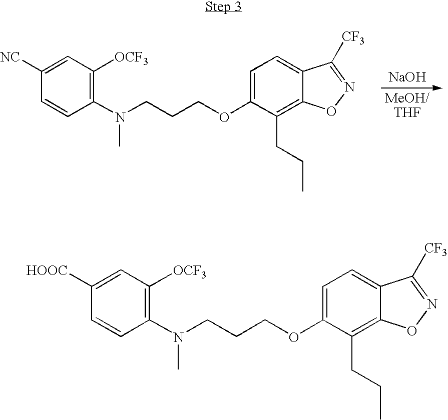
Aqueous NaOH (50%, 1.0 mL) was added to the nitrile (prepared according to the procedure of Example 15 Step 2, 60.0 mg, 0.12 mmol) in MeOH (5 mL) and THF (1.0 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac and the residue was chromatographed on silica gel using ethyl acetate:hexanes (30:70) 2.5% acetic acid to give the titled compound.
¹H NMR (CDCl₃) δ7.93 (m, 2H), 7.56 (d, 1H, J=9.0 Hz), 7.02 (m, 2H), 4.15 (t, 2H, J=6.0), 3.58 (t, 2H, J=7.5), 3.05 (s, 3H), 2.94 (t, 2H, J=7.5), 2.20 (m, 2H), 1.71 (m, 2H), 0.99 (t, 3H, J=7.5).
MS: m/z=521 (M+H).

### EXAMPLE 16

### Preparation of [2-oxo-1-(3-{[7-propyl-3-(trifluoromethyl)-1,2-benzisoxazol-6-yl]oxy}propyl)-1,2-dihydroguinolin-6-yl]acetic acid

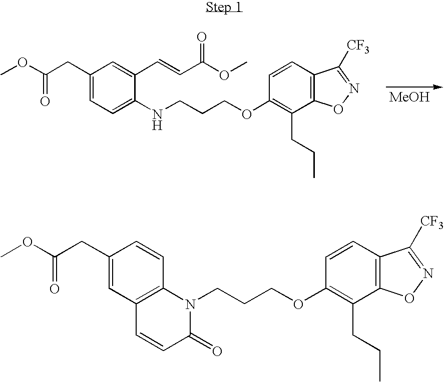 A solution of the indicated aniline (prepared according to the procedure of Example 10 Step 2, 10.0 mg, 0.02 mmol) in MeOH (2 mL) was heated to 60° C. overnight. The solvent was evaporated in vac and the residue was chromatographed on silica gel using ethyl acetate and hexanes (4:6) to give the titled compound. 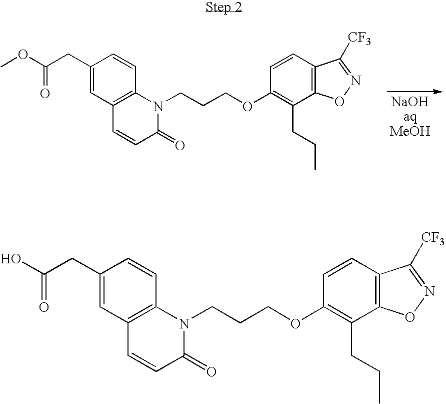
Aqueous NaOH (1 M, 28 μL) was added to a solution of the quinolone (prepared according to the procedure of Example 16 Step 1, 7.0 mg, 0.014 mmol) in MeOH (1.0 mL) and THF (0.5 mL). The resulting mixture was stirred at RT overnight. The reaction mixture was diluted with ethyl acetate and neutralized with acetic acid. The solvent was evaporated in vac and the residue was chromatographed on silica gel using ethyl acetate:hexanes (40:60) 2.5% acetic acid to give the titled compound.
¹H NMR (CDCl₃) δ7.70 (d, 1H, J=10.0 Hz), 7.60 (d, 1H, J=9), 7.53-7.40 (m, 3H), 7.10 (d, 1H, J=8.5), 6.75 (d, 1H, J=9.5), 4.57 (t, 2H, J=7.5), 4.28 (t, 2H, J=5.5), 3.77 (s, 2H), 3.00 (t, 2H, J=7.5), 2.35 (m, 2H), 1.78 (m, 2H), 1.00 (t, 3H, J=7.5).
MS: m/z=489 (M+H).

### EXAMPLE 17

### Preparation of 2-oxo-1-(3-{[7-propyl-3-(trifluoromethyl)-1,2-benzisoxazol-6-yl]oxy}propyl)indoline-5-carboxylic acid

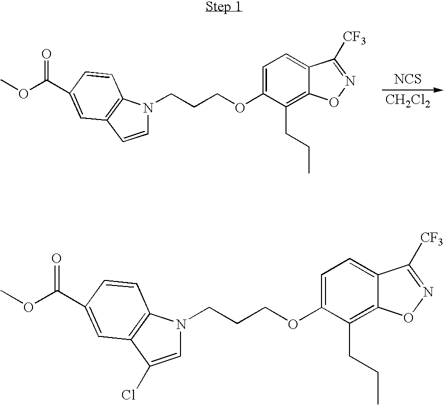 A solution of the indole methyl ester (prepared according to the procedure of Example 7, 0.19 g, 0.41 mmol) in CH₂Cl₂ (4 mL) was cooled to 0° C. N-chlorosuccinimide (20.0 mg, 0.15 mmol) was added and the resulting mixture was stirred for 30 minutes. Another addition of N-chlorosuccinimide (40.0 mg, 0.30 mmol) was made and the mixture was then stirred overnight. The solvent was evaporated and the residue was chromatographed on silica gel using ethyl acetate:hexanes (1:9) to give the titled compound. 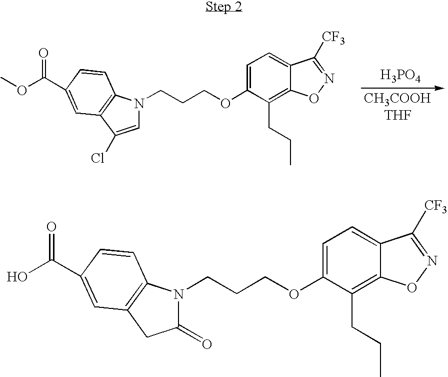
A mixture of the indicated chloro-indole (prepared according to the procedure of Example 17 Step 1, 129.5 mg, 0.26 mmol), H₃PO₄ (85%, 3 mL) and acetic acid (4 mL) in THF (3 mL) was heated to 100° C. overnight. The reaction mixture was poured into ice/H₂O and the mixture extracted with ethyl acetate. The combined ethyl acetate extracts were washed with brine, dried over sodium sulfate and filtered. The solvent was evaporated in vac and the residue was chromatographed on silica gel using, ethyl acetate:hexanes (40:60) 2.5% acetic acid to give the titled compound.
¹H NMR (CDCl₃) δ8.10 (d, 1H, J=8.0 Hz), 8.02 (s, 1H), 7.58 (d, 1H, J=8.5), 7.05 (d, 1H, J=8.5), 6.97 (d, 1H, J=8.5), 4.20 (t, 2H, J=6.0), 4.05 (t, 2H, J=7.0), 3.63 (s, 2H), 2.96 (t, 2H, J=7.5), 2.29 (m, 2H), 1.78 (m, 2H), 1.02 (t, 3H, J=7.5).
MS: m/z=463 (M+H).

### EXAMPLE 18

### Preparation of 2-methoxy-4-[methyl(3-{[7-propyl-3-(trifluoromethyl)-1,2-benzisoxazol-6-yl]oxy}propyl)amino]benzoic acid

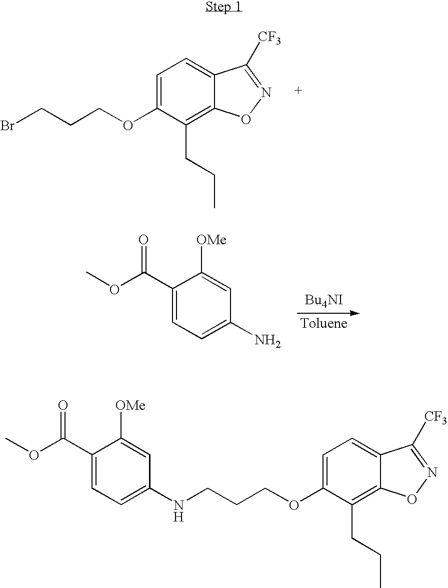 The indicated aniline (0.22 g, 20 mmol) was added to a solution of the bromide (prepared according to the procedure of Example 5, 0.46 g, 20 mmol) in toluene (12 mL) followed by Bu₄NI (0.45 g, 20 mmol) and Cs₂CO₃ (0.4 g, 20 mmol). The reaction mixture was heated at reflux overnight. The reaction mixture was cooled to room temperature and H₂O was added. The mixture was extracted with ethyl acetate (3×80 mL). The combined ethyl acetate extracts were washed with brine and dried over Na₂SO₄. Na₂SO₄ was then filtered off and solvent was evaporated in vac. The residue was chromatographed on silica gel using acetone:hexanes (20:80) 1% Et₃N to give the titled compound. 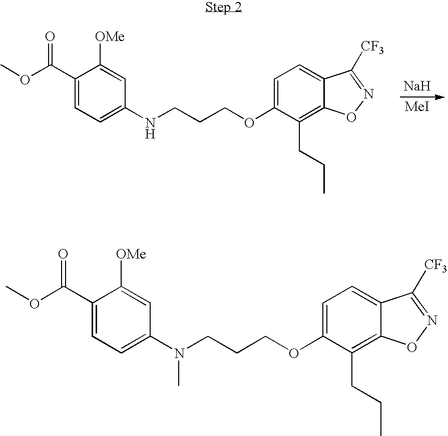
NaH (47.0 mg, 1.16 mmol) was added to a solution of the indicated aniline (prepared according to the procedure of Example 18 Step 2, 0.18 g, 0.39 mmol) in THF (5 mL) at −20° C. and stirred under N₂ for 10 minutes. MeI (0.12 mL, 1.94 mmol) was added to the reaction mixture and stirred at RT overnight. The resulting mixture was partitioned between ethyl acetate and H₂O. The combined ethyl acetate extracts were dried over sodium sulfate and filtered. The solvent was evaporated in vac to give the titled compound. 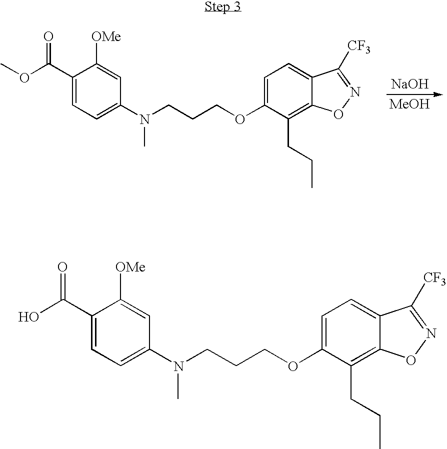
Aqueous NaOH (1N, 0.5 mL) was added to the aniline (prepared according to the procedure of Example 18 Step 2, 50.0 mg, 0.10 mmol) in MeOH (1 mL) and THF (1 mL). The resulting mixture was stirred at RT overnight. The reaction mixture was diluted with ethyl acetate and neutralized with acetic acid. The solvent was evaporated in vac and the residue was chromatographed on silica gel using, ethyl acetate:hexanes (30:70) 2.5% acetic acid to give the titled compound.
¹H NMR (CDCl₃) δ10.40 (br, 1H), 8.02 (d, 1H, J=9.0 Hz), 7.58 (d, 1H, J=8.5), 7.06 (d, 1H, J=8.5), 6.48 (m, 1H), 6.18 (m, 1H), 4.18 (t, 2H, J=6.0), 4.01 (s, 3H), 3.72 (t, 2H, J=7.5), 3.11 (s, 3H), 2.99 (t, 2H, J=7.5), 2.0 (m, 2H), 1.77 (m, 2H), 1.02 (t, 3H, J=7.0).
MS: m/z=467 (M+H).

### EXAMPLE 19

### Preparation of 4-[methyl(3-{[7-propyl-3-(trifluoromethyl)-1 2-benzisoxazol-6-yl]oxy}propyl)amino]-2-(trifluoromethyl)benzoic acid

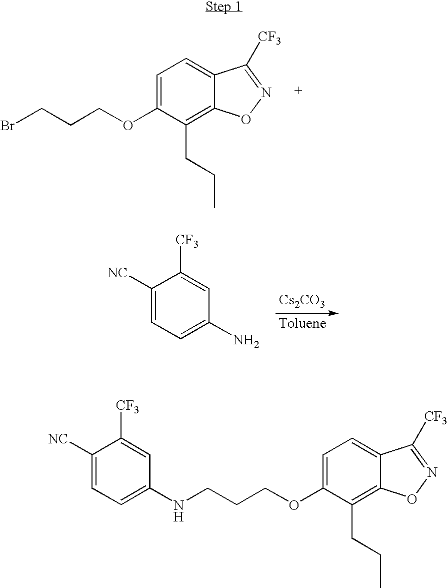 The indicated aniline (0.52 g, 2.8 mmol) was added to a solution of the bromide (prepared according to the procedure of Example 5, 0.21 g, 0.56 mmol) in Toluene (3 mL), followed by Cs₂CO₃ (0.18 g, 0.56 mmol). The reaction mixture was heated at reflux overnight. The reaction mixture was cooled to room temperature and H₂O was added. The resulting mixture was extracted with ethyl acetate (3×80 mL). The combined ethyl acetate extracts were washed with brine and dried over Na₂SO₄. Na₂SO₄ was then filtered off and solvent was evaporated in vac and the residue was chromatographed on silica gel using acetone:hexanes (20:80) 1% Et₃N to give the titled compound. 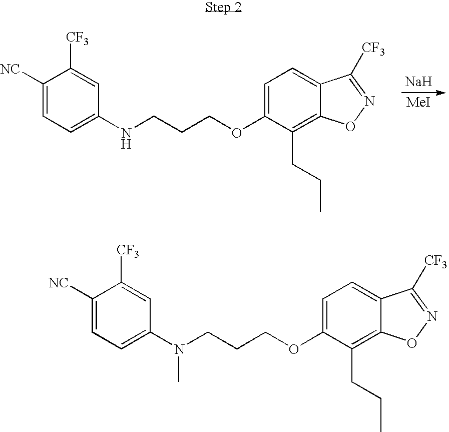
NaH (7.0 mg, 0.17 mmol) was added to the aniline (prepared according to the procedure of Example 19 Step 1, 41.0 mg, 0.09 mmol) in THF (1.5 mL) at −20° C. and stirred under N₂ for 10 minutes. MeI (16.3 μL, 0.26 mmol) was added to the reaction mixture and the resulting mixture stirred at room temperature overnight. The residue was partitioned between ethyl acetate and H₂O. The combined ethyl acetate extracts were dried over sodium sulfate and filtered. The solvent was evaporated in vac to give the titled compound. 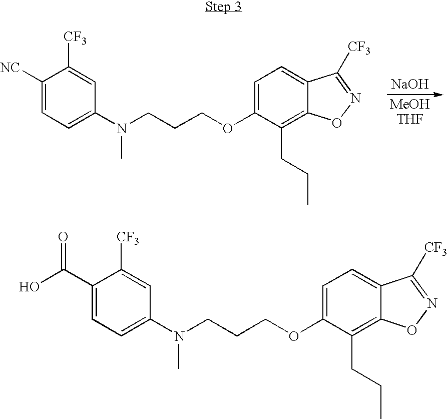
Aqueous NaOH (50, 2 mL) was added to the nitrile (prepared according to the procedure of Example 19 Step 2, 171.2 mg, 0.35 mmol) in MeOH (10 mL) and THF (4 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (20:80) 2.5% acetic acid to give the titled compound.
¹H NMR (CDCl₃) δ7.99 (d, 1H, J=9.0 Hz), 7.59 (d, 1H, J=9.0), 7.07 (m, 2H), 6.84 (m, 1H), 4.18 (t, 2H, J=5.5), 3.75 (t, 2H, J=7.5), 3.13 (s, 3H), 3.00 (t, 2H, J=7.5), 2.20 (m, 2H), 1.80 (m, 2H), 1.00 (t, 3H, J=7.5).
MS: m/z=505 (M+H).

### EXAMPLE 20

### Preparation of 2-fluoro-4-[methyl(3-{[7-propyl-3-(trifluoromethyl)-1,2-benzisoxazol-6-yl]oxy}propyl)amino]benzoic acid

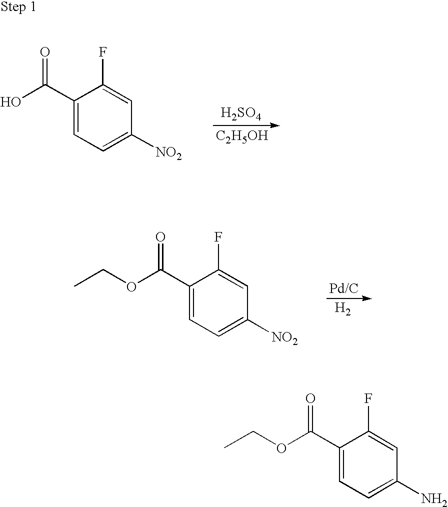 The 2-fluoro-4-nitrobenzoic acid (2.0 g, 10.8 mmol) was dissolved in C₂H₅OH (30 mL) with H₂SO₄ (conc. 3.5 mL) and heated at reflux overnight. The reaction mixture was poured into ice/H₂O (200 mL) and neutralized with NaHCO₃ (solid). The residue was extracted with ethyl acetate. The combined ethyl acetate extracts were washed with brine and dried over Na₂SO₄. Na₂SO₄ was then filtered off and solvent was evaporated in vac. The crude residue was dissolved in MeOH (30 mL) and reduced with Pd/C (10%, 0.2 g) under 1 atmosphere of H₂. The mixture was filtered and solvent was evaporated in vac to give the titled compound. 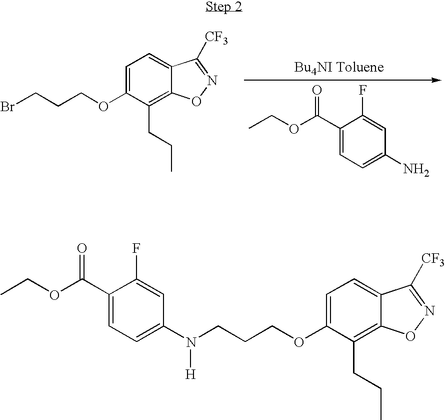
The indicated aniline (prepared according to the procedure of Example 20 Step 1, 0.22 g, 20 mmol) was added to a solution of the bromide (prepared according to the procedure of Example 5, 0.33 g, 0.89 mmol) in toluene (6 mL), followed by Bu₄NI (0.45 g, 20 mmol) and Cs₂CO₃ (0.29 g, 0.89 mmol). The reaction mixture and heated at reflux overnight. The reaction mixture was cooled to RT, H₂O was added and the resulting mixture extracted with ethyl acetate (3×80 mL). The combined ethyl acetate extracts were washed with brine and dried over Na₂SO₄. Na₂SO₄ was then filtered off and solvent was evaporated in vac. The residue was chromatographed on silica gel using acetone:hexanes (20:80) 1% Et₃N to give the titled compound. 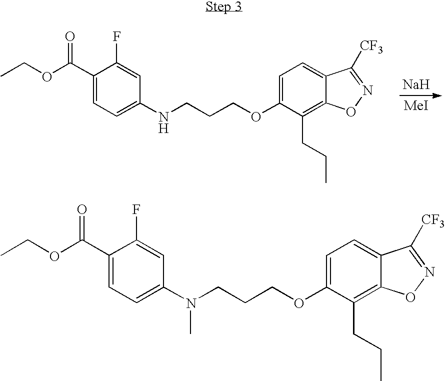
NaH (37.0 mg, 0.92 mmol) was added to the aniline (prepared according to the procedure of Example 20 Step 2, 0.14 g, 0.31 mmol) in THF (5 mL) at −20° C. and stirred under N₂ for 10 minutes. MeI (95.6 μL, 1.54 mmol) was added to the reaction mixture and stirred at RT overnight. The residue was partitioned between ethyl acetate and water. The combined ethyl acetate extracts were dried over sodium sulfate and filtered. The solvent was evaporated in vac to give the titled compound. 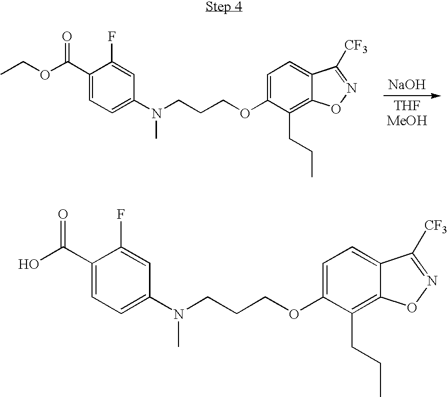
Aqueous NaOH (1N, 0.5 mL) was added to the aniline (prepared according to the procedure of Example 20 Step 3, 50.0 mg, 0.10 mmol) in MeOH (1 mL) and THF (1 mL). The mixture was stirred at RT overnight. The reaction mixture was diluted with ethyl acetate and neutralized with acetic acid. The solvent was evaporated in vac and the residue chromatographed on silica gel using ethyl acetate:hexanes (20:80) 2.5% acetic acid to give the titled compound.
¹H NMR (CDCl₃) δ7.87 (m, 1H), 7.58 (d, 1H, J=8.5 Hz), 7.06 (d, 1H, J=9.0), 6.53 (m, 1H), 6.61 (m, 1H), 4.18 (t,2H, J=5.5), 3.69 (t, 2H, J=7.0), 3.09 (s,3H), 3.00 (t, 2H, J=7.5), 2.20 (m, 2H), 1.71 (m, 2H), 1.03 (t, 3H, J=7.5).
MS: m/z=455 (M+H).

### EXAMPLE 21

### Preparation of 2-bromo-4-[methyl(3-{[7-[propyl-3-(trifluoromethyl)-1,2-benzisoxazol-6-yl]oxy}propyl)amino]benzoic acid

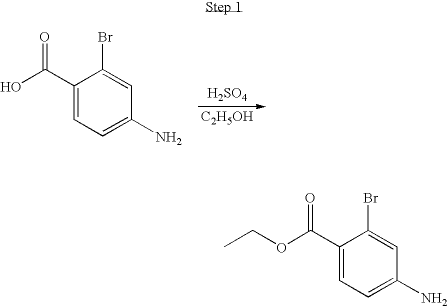

The 2-bromo-4-aminobenzoic acid (2.5 g, 11.5 mmol) was dissolved in C₂H₅OH (35 mL) with H₂SO₄ (conc., 3.5 mL). The mixture was heated at reflux overnight. The reaction mixture was poured into ice/H₂O (200 mL) and neutralized with NaHCO₃ (solid). The residue was extracted with ethyl acetate. The combined ethyl acetate extracts were washed with brine and dried over Na₂SO₄. Na₂SO₄ was then filtered off and solvent was evaporated in vac to give the titled compound. 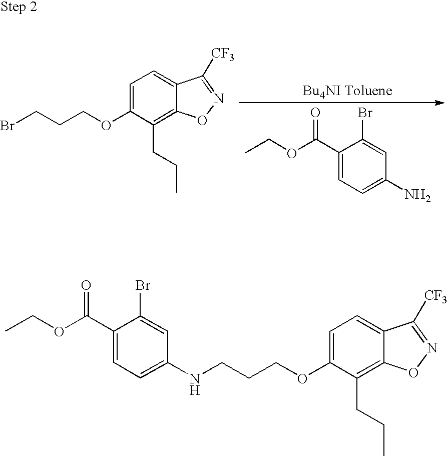
The indicated aniline (prepared according to the procedure of Example 21 Step 1, 0.25 g, 1.03 mmol) was added to a solution of the bromide (prepared according to the procedure of Example 5, 0.38 g, 1.03 mmol) in toluene (5 mL) followed by Bu₄NI (0.38 g, 1.03 mmol) and Cs₂CO₃ (0.34 g, 1.03 mmol). The reaction mixture was heated at reflux overnight. The reaction mixture was cooled to room temperature, H₂O was added and the resulting mixture extracted with ethyl acetate (3×80 mL). The combined ethyl acetate extracts were washed with brine and dried over Na₂SO₄. Na₂SO₄ was then filtered off and solvent was evaporated in vac. The residue was chromatographed on silica gel using: acetone:hexanes (20:80) 1% Et₃N to give the titled compound. 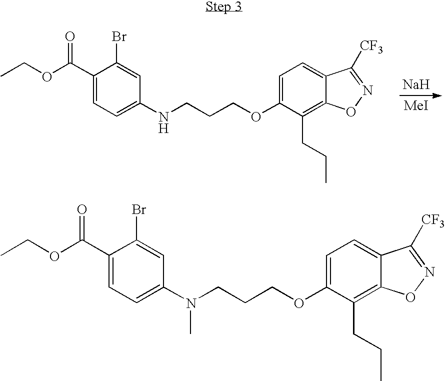
NaH (61.0 mg, 1.51 mmol) was added to the aniline (prepared according to the procedure of Example 21 Step 2, 0.27 g, 0.50 mmol) in THF (5 mL) at −20° C. and stirred under N₂ for 10 minutes. Mel (0.16 mL, 2.52 mmol) was added to the reaction mixture and stirred at RT overnight. The residue was partitioned between ethyl acetate and H₂O. The combined ethyl acetate extracts were dried over sodium sulfate and filtered. The solvent was evaporated in vac to give the titled compound. 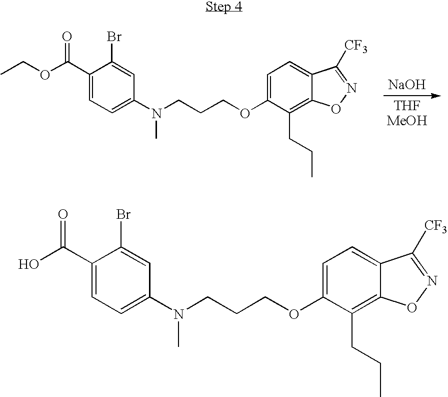
Aqueous NaOH (1N, 0.5 mL) was added to the aniline (prepared according to the procedure of Example 21 Step 2, 50.0 mg, 0.10 mmol) in MeOH (1 mL) and THF (1 mL) and stirred at RT overnight. The reaction mixture was diluted with ethyl acetate and neutralized with acetic acid. Then the solvent was evaporated in vac and the residue chromatographed on silica gel using ethyl acetate:hexanes (20:80) 2.5% acetic acid to give the titled compound.
¹H NMR (CDCl₃) δ7.97 (d, 1H, J=9.0 Hz), 7.57 (d, 1H, J=9.0), 7.06 (d, 1H, J=9.0), 6.96 (m, 1H), 6.66 (m, 1H), 4.17 (t, 2H, J=5.5), 3.68 (t, 2H, J=7.5), 3.07 (s, 3H), 3.00 (t, 2H, J=7.5), 2.19 (m, 2H), 1.80 (m, 2H), 1.03 (t, 3H, J=7.5).
MS: m/z=517 (M+H).

### EXAMPLE 22

### Preparation of 6-[methyl(3-{[7-propyl-3-(trifluoromethyl)-1,2-benzisoxazol-6-yl]oxy}propyl)amino]nicotinic acid

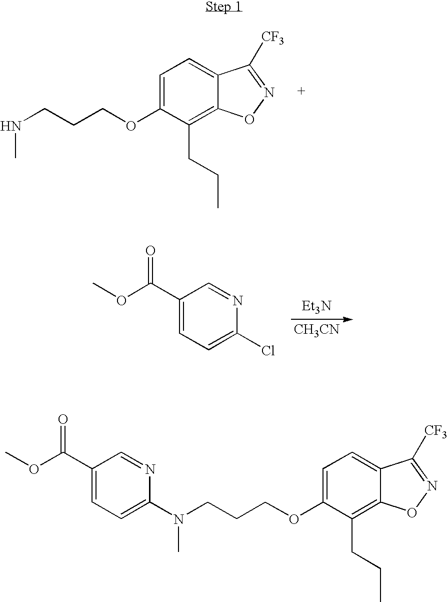 Methyl 6-chloronicotinate (70 mg, 0.45 mmol) and Et₃N (0.57 mL, 4.1 mmol) were added to a solution of the amine (prepared according to the procedure of Example 6, 0.43 g, 1.36 mmol) in CH₃CN (10 mL). The reaction mixture was heated at reflux overnight. The solvent was evaporated in vac and the residue was chromatographed on silica gel using ethyl acetate:hexanes (1:4) to give the titled compound. 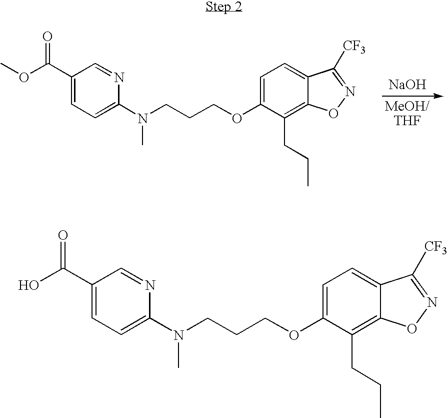
Aqueous NaOH (1N, 1.6 mL) was added to the aniline (prepared according to the procedure of Example 22 Step 1, 179.5 mg, 0.40 mmol) in MeOH (6.4 mL) and THF (1.1 mL). The mixture was stirred at RT overnight. The reaction mixture was diluted with ethyl acetate and neutralized with acetic acid. The solvent was evaporated in vac and the residue was chromatographed on silica gel using ethyl acetate: hexanes (30:70) 2.5% acetic acid to give the titled compound.
¹H NMR (CDCl₃) δ8.87 (s, 1H), 8.06 (d, 1H, J=9.0 Hz), 7.58 (d, 1H, J=9.0), 7.05 (d, 1H, J=9.0),), 6.54 (d, 1H, J=9.5), 4.19 (t, 2H, J=6.0), 3.92 (t, 2H, J=7.0), 3.19 (s, 3H), 2.23 (m, 24H), 1.78 (m, 2H), 1.03 (t, 3H, J=7.5).
MS: m/z=438 (M+H).

### EXAMPLE 23

### Preparation of {6-[methyl(3-{[7-propyl-3-(trifluoromethyl)-1,2-benzisoxazol-6-yl]oxy}propyl)amino]pyridin-3-yl}acetic acid

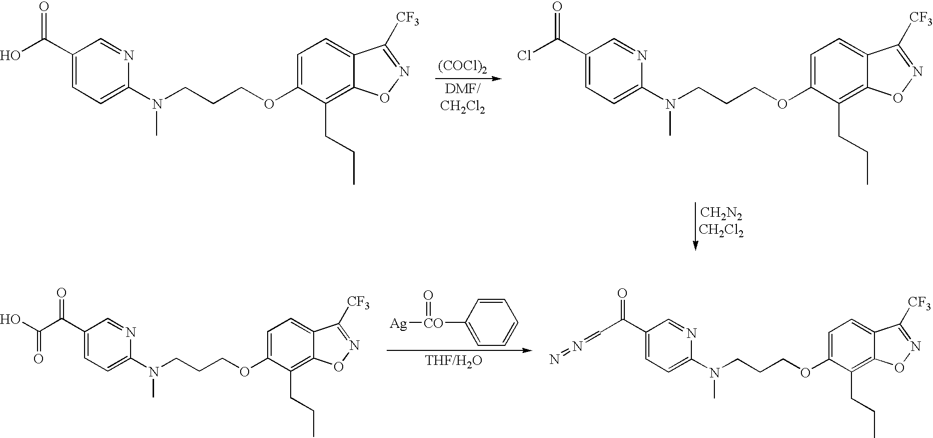 Oxalyl chloride (7.2 μL, 0.08 mmol) and DMF (2 drops, catalytic) were added to the aminopyridine (prepared according to the procedure of Example 22 Step 2, 31.1 mg, 0.07 mmol) in CH₂Cl₂ (1.2 mL). The mixture was stirred at RT for 3 hours. The solvent was evaporated in vacuo. The residue was dissolved in CH₂Cl₂ (1 mL), CH₂N₂ in ether (excess) was added and the mixture was stirred for 3 hours. The solvent was evaporated in vacuo and the residue was dissolved in THF (0.48 mL) and H₂O (0.24 mL). Silver benzoate (24.5 mg, 0.1 mmol) was added and the reaction mixture was heated at 70° C. overnight. The solvent was evaporated in vac and the residue was purified by prep RP-18 HPLC purification (acetonitrile:H₂O 15 minute gradient 10 to 100%:0.1% trifluoroacetic acid) to afford the titled compound.
¹H NMR (CDCl₃) δ8.78 (s, 1H), 8.06 (d, 1H, J=9.0 Hz), 7.58 (d, 1H, J=9.0), 7.05 (d, 1H, J=9.0), 6.54 (d, 1H, J=9.5), 4.19 (t, 2H, J=6.0), 3.92 (m, 4H), 3.19 (s, 3H), 2.23 (m, 2H), 1.78 (m, 2H), 1.03 (t, 3H, J=7.5).
MS: m/z=452 (M+H).

### EXAMPLE 24

### Preparation of 5-[methyl(3-{[7-propyl-3-(trifluoromethyl)-1,2-benzisoxazol-6-yl]oxy}propyl)amino]pyrazine-2-carboxylic acid

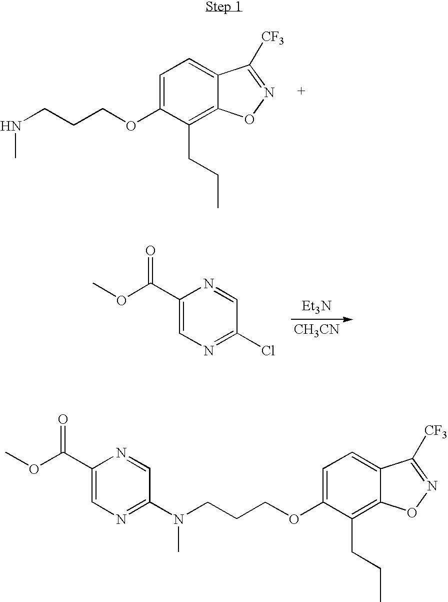

The indicated chloropyrazine (202.0 mg, 1.17 mmol) and Et₃N (1.63 mL, 11.7 mmol) were added to a solution of the amine (prepared according to the procedure of Example 6, 1.48 g, 4.68 mmol) in CH₃CN (10 mL). The reaction mixture was heated at reflux overnight. The solvent was evaporated in vac and the residue was chromatographed on silica gel using acetone:hexanes (3:7) to give the titled compound. 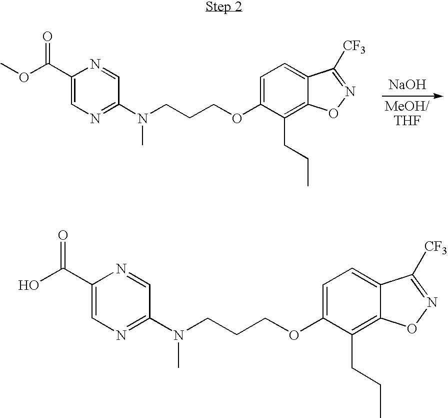
Aqueous NaOH (1N, 1.0 mL) was added to the aminopyrazine (prepared according to the procedure of Example 24 Step 1, 110.03 mg, 0.24 mmol) in MeOH (1.5 mL) and THF (1.0 mL). The mixture was stirred at RT overnight. The reaction mixture was diluted with ethyl acetate and neutralized with acetic acid. The solvent was evaporated in vac and the residue was chromatographed on silica gel using ethyl acetate:hexanes (70:30) 2.5% acetic acid to give the titled compound.
¹H NMR (CDCl₃) δ8.60 (br, 1H), 8.06 (m, 2H), 7.58 (d, 1H, J=9.0 Hz), 7.05 (d, 1H, J=8.5), 4.17 (t, 2H, J=5.5), 3.83 (t, 2H, J=7.0), 3.15 (s, 3H), 2.97 (t, 2H, J=7.5), 2.19 (m, 2H), 1.77 (m, 2H), 1.01 (t, 3H, J=7.5).
MS: m/z=439 (M+H).

### EXAMPLE 25

### Preparation of {5-[methyl(3-{[7-propyl-3-(trifluoromethyl)-1,2-benzisoxazol-6-yl]oxy}propyl)amino]pyrazin-2-yl}acetic acid

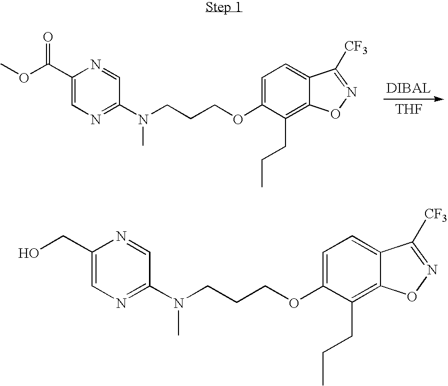 A solution of the pyrazine ester (prepared according to the procedure of Example 24 Step 1, 0.2 g, 40.44 mmol) in THF (10 mL) was cooled to −30° C. DIBAL (1 M Hexanes, 2.2 mL, 2.2 mmol) was added and the reaction mixture allowed to warm to RT over ˜2 hours. The reaction mixture was quenched with NaOH (1 N aqueous, 20 mL) and partitioned between ethyl acetate and water. The combined ethyl acetate extracts were dried over Na₂SO₄ and evaporated in vac to give the titled compound. 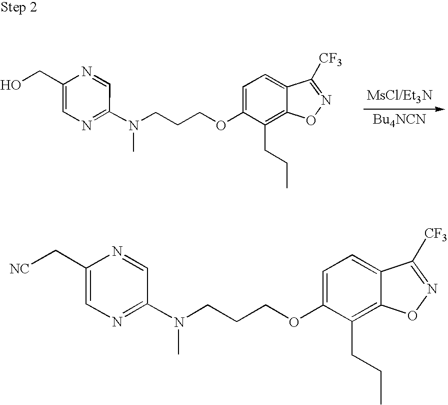
The indicated alcohol (prepared according to the procedure of Example 25 Step 1, 0.14 g, 0.33 mmol), Et₃N (0.14 mL, 1.0 mmol) and MsCl (31.0 μL, 0.4 mmol) were combined in THF (2 mL) under N₂ and stirred at −30° C. for 10 minutes. Bu₄NCN (0.56 g, 1.98 mmol) was added to the reaction, the acetone-dry ice bath was removed and the reaction stirred vigorously for 3 hours. The reaction mixture was quenched with NaOH aq (1N, 30 mL) and partitioned between ethyl acetate and water. The combined ethyl acetate extracts were dried over sodium sulfate and filtered. The solvent was evaporated in vac and the residue was chromatographed on silica gel using acetone and hexanes (3:7) to give the titled compound. 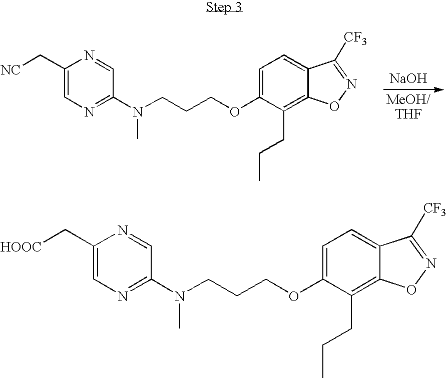
Aqueous NaOH (50%, 1 mL) was added to the nitrile (prepared according to the procedure of Example 25 Step 2, 35.0 mg, 0.08 mmol) in MeOH (4 mL) and THF (2 mL). The reaction mixture was heated at reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off and solvent was evaporated in vac. The residue was chromatographed on silica gel using ethyl acetate:hexanes (50:50) 2.5% acetic acid to give the titled compound.
¹H NMR (CDCl₃) δ8.60 (br, 1H), 8.06 (m, 2H), 7.58 (d, 1H, J=9.0 Hz), 7.05 (d, 1H, J=8.5), 4.17 (t, 2H, J=5.5), 3.83 (t, 2H, J=7.0), 3.76 (s, 2H), 3.15 (s, 3H), 2.97 (t, 2H, J=7.5), 2.19 (m, 2H), 1.77 (m, 2H), 1.01 (t, 3H, J=7.5).
MS: m/z=453 (M+H).

### EXAMPLE 26

### Preparation of 2-[methyl(3-{[7-propyl-3-(trifluoromethyl)-1,2-benzisoxazol-6-yl]oxy}propyl)amino]pyrimidine-5-carboxylic acid

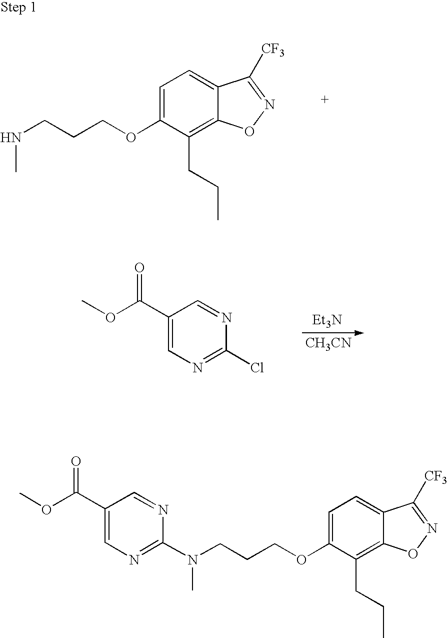 The indicated chloropyrimidine (0.11 g, 0.63 mmol) and Et₃N (0.88 mL, 6.3 mmol) were added to a solution of the amine (prepared according to the procedure of Example 6, 0.8 g, 2.52 mmol) in CH₃CN (5 mL). The reaction mixture was heated at reflux overnight. The solvent was evaporated in vac and the residue was chromatographed on silica gel using ethyl acetate:hexanes (2:8) to give the titled compound. 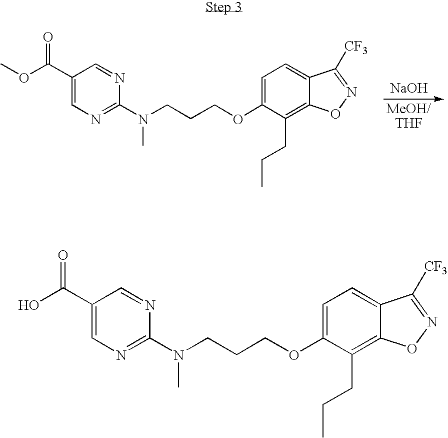
Aqueous NaOH (1N, 90.0 μL) was added to the aminopyrimidine (prepared according to the procedure of Example 26 Step 2, 20.0 mg, 0.24 mmol) in MeOH (1.0 mL) and THF (0.5 mL). The mixture was stirred at RT overnight. The reaction mixture was diluted with ethyl acetate and neutralized with acetic acid. The solvent was evaporated in vac and the residue chromatographed on silica gel using ethyl acetate:hexanes (40:60) 2.5% acetic acid to give the titled compound.
¹H NMR (CDCl₃) δ8.85 (m, 2H), 7.58 (d, 1H, J=8.5 Hz), 7.05 (d, 1H, J=9.0), 4.19 (t, 2H, J=5.5), 3.99 (t, 2H, J=7.0), 3.32 (s, 3H), 2.97 (t, 2H, J=7.5), 2.24 (m, 2H), 1.77 (m, 2H), 1.02 (t, 3H, J=7.5).
MS: m/z=439 (M+H).

### EXAMPLE 27

### Preparation of {2-[methyl(3-{[7-propyl-3-(trifluoromethyl)-1,2-benzisoxazol-6-yl]oxy}propyl)amino]pyrimidin-5-yl}acetic acid

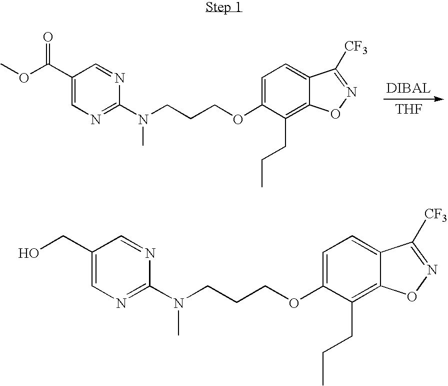 A solution of the pyrimidine ester (prepared according to the procedure of Example 26 Step 1, 0.25 g, 0.55 mmol) in THF (10 mL) was cooled to −30° C. DIBAL (1 M hexanes, 2.74 mL, 2.74 mmol) was added and reaction warm to RT over ˜2 hours. The reaction mixture was quenched with NaOH (1 N aqueous, 20 mL) and partitioned between ethyl acetate and water. The ethyl acetate extracts were dried over Na₂SO₄ and were evaporated in vac to give the titled compound. 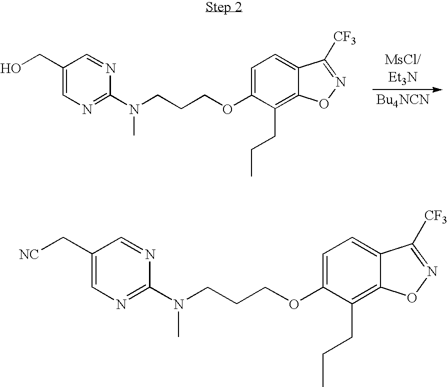
The alcohol (prepared according to the procedure of Example 27 Step 1, 0.20 g, 0.55 mmol), Et₃N (0.23 mL, 1.64 mmol) and MsCl (51.0 μL, 0.66 mmol) were combined in THF (5 mL) under N₂ and stirred at −30° C. for 10 minutes. Bu₄NCN (0.72 g, 3.29 mmol) was added to the reaction, the acetone-dry ice bath was removed and the reaction was stirred vigorously for 3 hours. The reaction mixture was quenched with NaOH aq (1N, 40 mL) and partitioned between ethyl acetate and water. The combined ethyl acetate extracts were dried over sodium sulfate and filtered. The solvent was evaporated in vac and the residue was chromatographed on silica gel using acetone and hexanes (2:8) to give the titled compound. 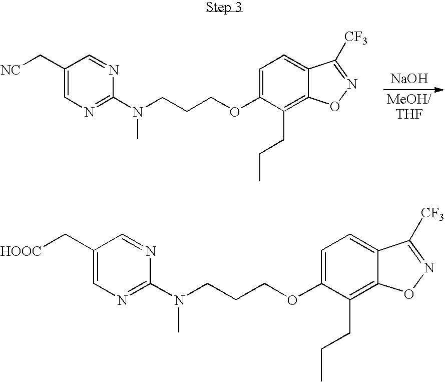
The nitrile (prepared according to the procedure of Example 27 Step 2. 116.3 mg, 0.27 mmol) was dissolved in MeOH/THF (85:15). Aqueous NaOH (50%, 2.5 ml) was added and the reaction mixture was heated to reflux overnight. The reaction mixture was cooled to room temperature and neutralized with acetic acid, then extracted with ethyl acetate. The combined ethyl acetate extracts were washed with brine and dried over Na₂SO₄. Na₂SO₄ was then filtered off, solvent was evaporated in vac and the residue was purified by chromatography on silica gel using ethyl acetate:hexanes (30:70) 2.5% acetic acid to give the titled compound.
¹H NMR (CDCl₃) δ8.23 (s, 2H), 7.57 (d, 1H, J=8.5 Hz), 7.05 (d, 1H, J=9.0), 4.19 (t, 2H, J=5.5), 3.99 (t, 2H, J=7.0), 3.48 (s, 2H), 3.32 (s, 3H), 2.97 (t, 2H, J=7.5), 2.24 (m, 2H), 1.77 (m, 2H), 1.02 (t, 3H, J=7.5).
MS: m/z=453 (M+H).

### EXAMPLE 28

### Preparation of {2-[methyl(3-{[7-propyl-3-(trifluoromethyl)-1,2-benzisoxazol-6-yl]oxy}propyl)amino]-1,3-thiazol-4-yl}acetic acid

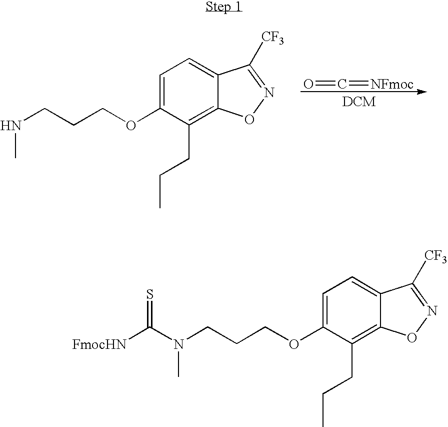

The amine (prepared according to the procedure of Example 6 (0.4 g, 1.27 mmol) was dissolved in dichloromethane (2.5 mL). FMOC isothiocyanate (0.356 g, 1.27 mmol) was added and the reaction mixture stirred for 4 hours at room temperature. The solvent was removed followed by silica gel chromatography (ethyl acetate:hexanes (2:8)) to give the titled compound.

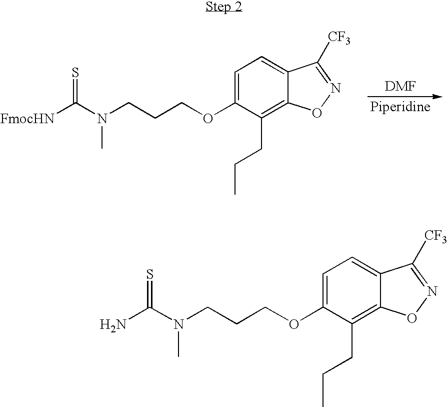

Piperidine (0.8 ml) was added to the thiourea (prepared according to the procedure of Example 28 Step 1, 0.3 g, 0.50 mmol) in DMF (3.2 mL) and the solution stirred overnight. The mixture was poured into saturated sodium bicarbonate followed by aqueous/ethyl acetate work-up and silica gel chromatography (acetone:hexanes (3:7)) to give the titled compound.

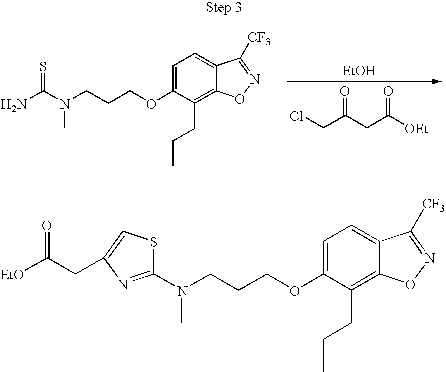

The thiourea (prepared according to the procedure of Example 28 Step 2, 0.04 g, 0.106 mmol) was dissolved in absolute ethanol (1.0 ml) and ethyl 4-chloroacetoacetate was added (0.018 g, 0.106 mmol). The mixture was heated to reflux for 1 hour, then cooled to room temperature. The solution was poured into saturated sodium bicarbonate followed by aqueous/ethyl acetate work-up and silica gel chromatography (acetone:hexanes (3:7)) to give the titled compound.

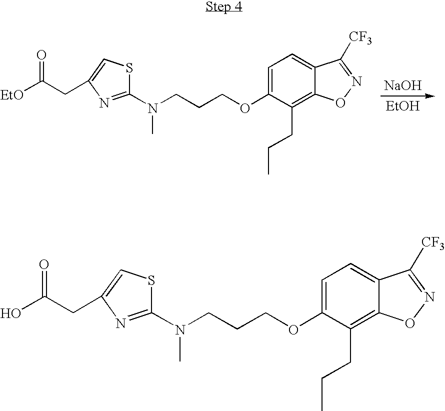

To the thiazole (prepared according to the procedure of Example 28 Step 3, 18.0 mg, 0.24 mmol) in ethanol (2.0 mL) was added sodium hydroxide (aq) (1N, 2 ml) and the resulting mixture was stirred at room temperature overnight. The reaction mixture was poured into water, acidified with acetic acid followed by aqueous/ethyl acetate work-up and silica gel chromatography (acetone:hexanes (3:7:1% acetic acid)) to give the titled compound.

NMR (CD₃OD) δ7.63 (d, 1H, J=8.9 Hz), 7.27 (d, 1H, J=8.8 Hz), 6.31 (s, 11H), 4.21 (t, 2H, J=5.9 Hz), 3.69 (t, 2H, J=7.2 Hz), 3.44 (s, 2H), 3.06 (s, 3H), 2.92 (t, 2H, J=7.6 Hz), 1.88 (br s, 4H), 1.70 (m, 2H), 0.94 (t, 3H, J=7.4 Hz).

MS: m/z=458 (M+H).

### EXAMPLE 29

### Preparation of 2-[methyl(3-{[7-propyl-3-(trifluoromethyl)-1,2-benzisoxazol-6-yl]oxy}propyl)amino]-1,3-thiazole-5-carboxylic acid

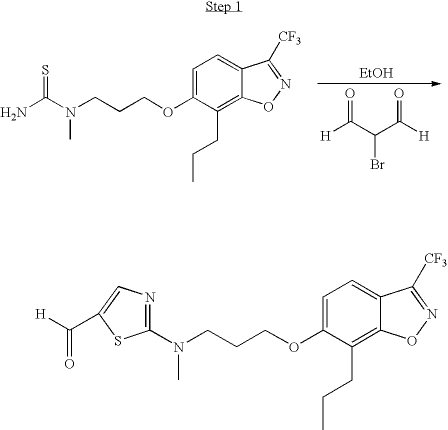

The thiourea (prepared according to the procedure of Example 28 Step 2, 0.5 g, 0.266 mmol) was dissolved in absolute ethanol (1.5 ml) and bromomalonaldehyde was added (0.042 g, 0.28 mmol). The mixture was heated to reflux for 1 hour. The solution was poured into saturated sodium bicarbonate followed by aqueous/ethyl acetate work-up and silica gel chromatography (acetone:dichloromethane (5:95:0.5% triethylamine)) to give the titled compound.

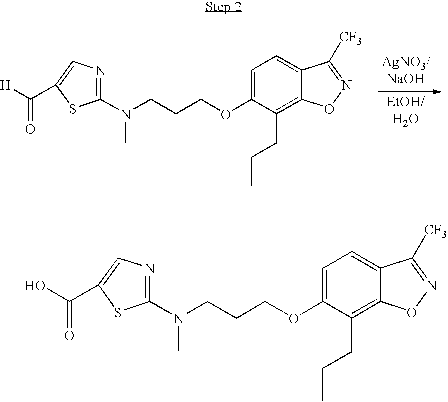

A flask was charged with silver nitrate (0.020 g, 0.118 mmol) and water (0.3 ml). Sodium hydroxide was added (0.010 g, 0.236 mmol) in 0.25 ml of water. The solution was cooled to 0° C., followed by addition of the aldehyde (prepared according to the procedure of Example 29 Step 1, 0.025 g, 0.059 mmol), in ethanol (0.5 ml) and stirred for 2 days. Solids were removed by filtration, the solvent removed in vacuo, and purified by RP-18 HPLC purification (acetonitrile: H₂O 15 minute gradient 10 to 100%:0.1% trifluoroacetic acid) to give the titled compound.

NMR (DMSO-d6) δ7.74 (d, 1H, J=8.8), 7.71 (s, 1H), 7.33 (d, 1H, J=8.9), 4.21 (t, 2H, J=5.7), 3.99 (t, 2H, J=7.0), 3.10 (s, 3H), 2.89 (t, 2H, J=7.3), 2.12 (m, 2H), 1.66 (m, 2H), 0.93 (t, 3H, J=7.4).

MS: m/z=444.3 (M+H).

### EXAMPLE 30

### Preparation of {2-[methyl(3-{[7-propyl-3-(trifluoromethyl)-1,2-benzisoxazol-6-yl]oxy}propyl)amino]-1,3-thiazol-5-yl}acetic acid

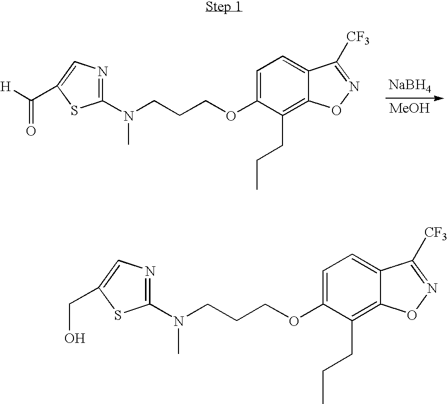

The aldehyde (prepared according to the procedure of Example 29 Step 1, 0.070 g, 0.164 mmol) was dissolved in methanol (2.0 ml), sodium borohydride was added (0.010 g, 0.164 mmol) and the mixture stirred for 1 hour. The reaction mixture was poured into brine followed by aqueous/ethyl acetate work-up to give the titled compound.

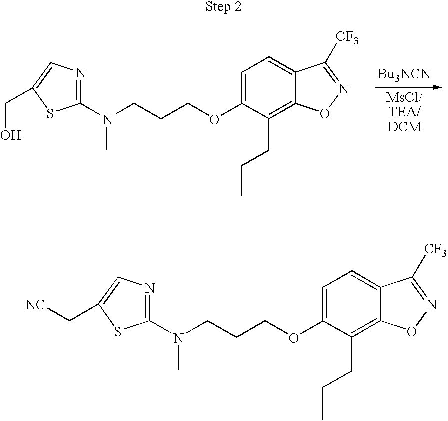

The alcohol (prepared according to the procedure of Example 30 Step 1, 0.37 g, 0.85 mmol) was dissolved in dichloromethane (4.3 mL) and the solution cooled to −40° C. Triethylamine (0.259 g, 2.56 mmol) and methanesulfonyl chloride (0.12 g, 1.109 mmol) were added and the solution stirred for 10 minutes. Tetrabutylammonium cyanide (0.72 g, 3.29 mmol) was added and the cooling bath removed. The mixture was stirred vigorously overnight, poured into saturated sodium bicarbonate followed by aqueous/ethyl acetate work-up and silica gel chromatography (acetone:dichloromethane (5:95:0.5% triethylamine)) followed by a second silica gel chromatography (dichloromethane:hexanes (80:20)) to give the titled compound.

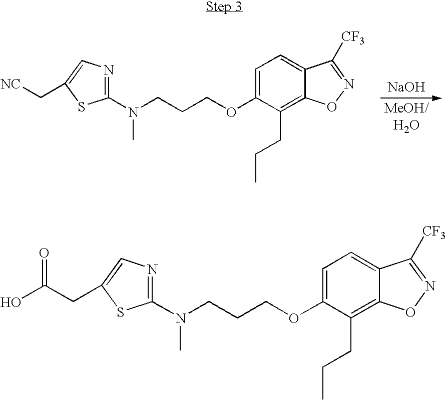

To the nitrile (prepared according to the procedure of Example 30 Step 2, 140.0 mg, 0.32 mmol) in methanol (4.8 mL) was added sodium hydroxide (aq) (0.128 g, 3.20 mmol, 2 ml water) and the resulting solution heated at reflux for 1 hour. Additional sodium hydroxide (190 mg, 4.9 mmol, 1.6 ml water) was added and reflux continued for 1 hour. The reaction mixture was cooled to room temperature poured into water, acidified with acetic acid, followed by aqueous/dichlormethane work-up and two silica gel chromatographies (acetone:hexanes (3:7:1% acetic acid) and (ethyl acetate:hexanes (90:10:1% acetic acid) to give the titled compound.

NMR (CD₃OD) δ7.63 (d, 1H, J=8.8 Hz), 7.25 (d, 1H, J=8.9 Hz), 6.89 (s, 111), 4.22 (t, 2H, J=5.9 Hz), 3.69 (t, 2H, J=7.2 Hz), 3.63 (s, 2H), 3.10 (s, 3H), 2.96 (t, 2H, J=7.4 Hz), 2.96 (t, 2H, J=7.4 Hz), 2.20 (m, 2H), 1.73 (m, 2H), 0.98 (t, 3H, J=7.5 Hz).

MS: m/z=458.3 (M+H).

### EXAMPLE 31

### Preparation of [1-(3-{[7-propyl-3-(trifluoromethyl)-1,2-benzisoxazol-6-yl]oxy}propyl)-1H-indol-5-yl]acetic acid

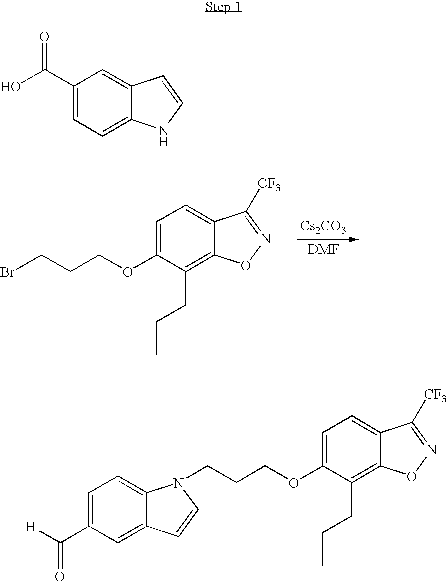

The bromide was prepared according to the procedure of Example 5. To a solution of this bromide (0.1 g, 0.273 mmol) in DMF (1.1 mL) was added methyl indole-5-carboxyaldehyde (0.079 g, 0.546 mmol) and cesium carbonate (0.177 g, 0.546 mmol). The reaction mixture stirred at room temperature for 2 hours. Water was added followed by aqueous/ethyl acetate work-up and silica gel chromatography (acetone:hexanes (3:7)) to give the titled compound.

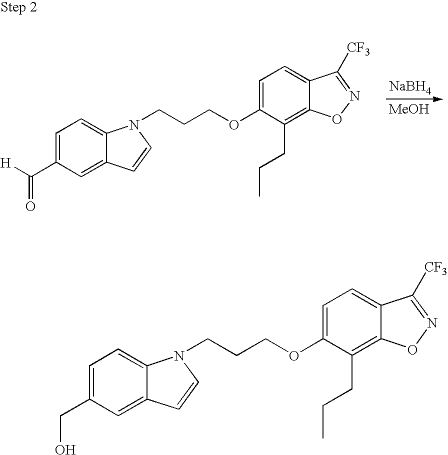

The aldehyde (prepared according to the procedure of Example 31 Step 1, 0.041 g, 0.095 mmol) was dissolved in methanol (1 ml), and sodium borohydride was added (0.004 g, 0.095 mmol). The reaction mixture was stirred for 1 hour, poured into 1 N sodium hydroxide (20 ml) followed by aqueous/ethyl acetate work-up to give the titled compound.

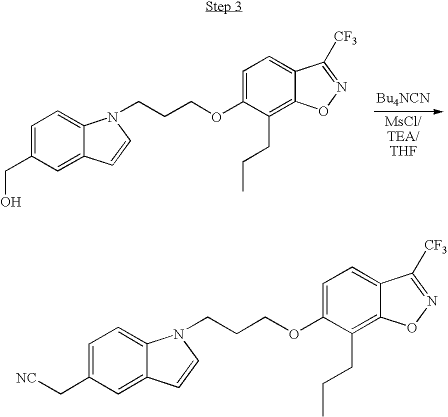

The alcohol (prepared according to the procedure of Example 31 Step 2, 0.050 g, 0.12 mmol) in THF (0.6 mL) was cooled to −20° C. Triethylamine (0.035 g, 0.35 mmol) and methanesulfonyl chloride (0.016 g, 0.14 mmol) were added and the resulting solution stirred for 10 minutes. Tetrabutylammonium cyanide (0.19 g, 0.70 mmol) was added the cooling bath removed, and the solution stirred 2.5 hours. The mixture was poured into saturated sodium bicarbonate followed by aqueous/ethyl acetate work-up and silica gel chromatography (acetone:hexanes (3:7)) to give the titled compound.

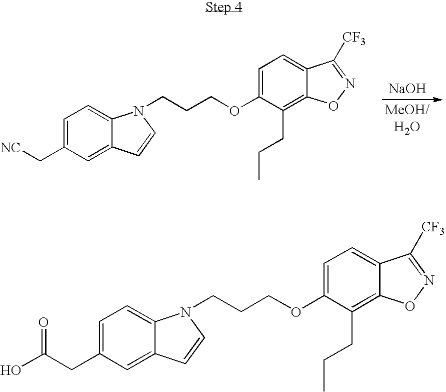

To the nitrile (prepared according to the procedure of Example 31 Step 3, 20.0 mg, 0.045 mmol), in methanol (1.5 mL) was added sodium hydroxide (25% (wt., aq) (0.5 ml) and stirred at reflux for 6 hours. The mixture was cooled to room temperature, poured into water, acidified with acetic acid, followed by aqueous/dichlormethane work-up and RP-18 HPLC purification (acetonitrile: H₂O 15 minute gradient 10 to 100%:0.1% trifluoroacetic acid) to give the titled compound.

NMR (CD₃OD) δ7.57 (d, 1H, J=8.7 Hz), 7.45 (s, 1H), 7.34 (d, 1H, J=8.5 Hz), 7.16 (d, 1H, J=3.2 Hz), 7.11 (d, 1H, J=8.8 Hz), 7.03 (d, 1H, J=9.9 Hz), 6.39 (d, 1H, J=3.2 Hz), 4.41 (t, 2H, J=6.6 Hz), 4.05 (t, 2H, J=5.8 Hz), 3.63 (s, 2H), 2.96 (t, 2H, J=7.4 Hz), 2.33 (m, 2H), 1.76 (m, 2H), 0.99 (t, 3H, J=7.4 Hz).

MS: m/z=461.3 (M+H).

### EXAMPLE 32

### Preparation of [1-(3-{[7-propyl-3-(trifluoromethyl)-1,2-benzisoxazol-6-yl]oxy}propyl)-1H-benzimidazol-5-yl]acetic acid

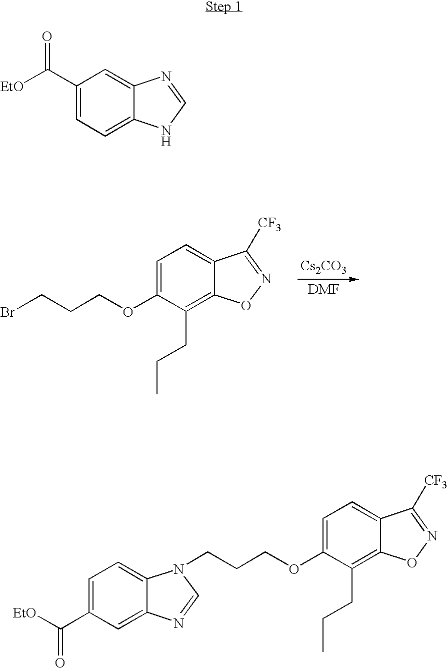

The bromide (prepared according to the procedure of Example 5, 0.2 g, 0.546 mmol) and ethyl 1H-benzimidazole-5-carboxylate (0.208 g, 1.093 mmol) were dissolved in DMF (3.0 mL) and cesium carbonate (0.355 g, 1.093 mmol) was added. The reaction mixture was stirred at room temperature overnight, water was then added, followed by aqueous/ethyl acetate work-up and silica gel chromatography (CH₃CN:ethyl acetate (1:19)) to give the titled compound. 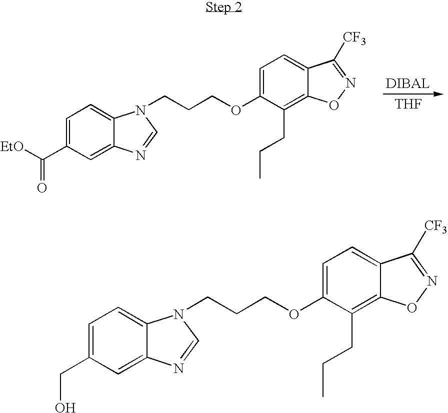
The ester (prepared according to the procedure of Step 1, 0.133 g, 0.28 mmol) was dissolved in THF (6 ml) and cooled to −20° C. Diisobutyl aluminum hydride (1.0 M in hexanes) was added (1.4 ml, 1.4 mmol) and the reaction mixture stirred for 1 hour −20° C., after which the mixture was allowed to warm to 0° C. The reaction mixture was poured into 1 N NaOH (20ml) followed by aqueous/dichloromethane work-up to give the titled compound.

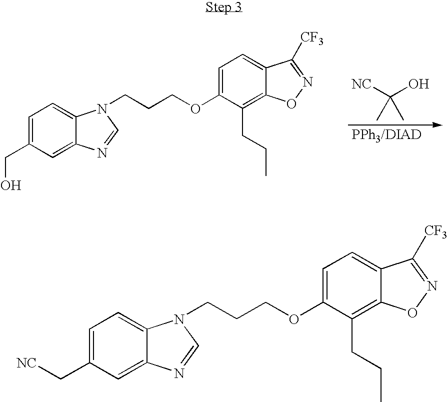

The benzimidazole alcohol (prepared according to the procedure of Step 2, 0.05 g, 0.12 mmmol) was dissolved in 6:4 tetrahydrofuran:benzene (1 mL). Acetone cyanohydrin (10 mg, 0.115 mmol), triphenyl phosphine (0.039 g, 0.15 mmol), and diisopropyl azodicarboxylate (30 mg, 0.150 mmol) were added and the solution stirred overnight. The solvent was removed in vac. followed by RP-18 HPLC purification (acetonitrile:H₂O 15 minute gradient 10 to 100%:0.1% trifluoroacetic acid) and silica gel chromatography (ethyl acetate) to give the titled compound.

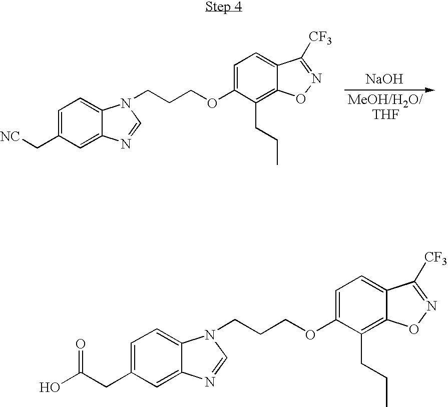

To the nitrile (prepared according to the procedure of Step 3, 20.0 mg, 0.045 mmol) in methanol (1.5 mL) was added sodium hydroxide (25% (wt., aq) (0.5 ml) and tetrahydrofuran (0.3 ml). The mixture was heated under reflux for 5 hours. The reaction mixture was cooled to room temperature, poured into water, acidified with acetic acid, followed by aqueous/dichlormethane work-up and RP-18 HPLC purification (acetonitrile: H₂O 15 minute gradient 10 to 100%:0.1% trifluoroacetic acid) to give the titled compound.

NMR (CD₃OD) δ9.48 (s, 1H), 7.91 (d, 1H, J=8.6 Hz), 7.80 (s, 1H), 7.62 (d, 1H, J=8.8 Hz), 7.60 (d, 1H, J=7.4 Hz), 7.23 (d, 1H, J=8.9 Hz), 4.79 (t, 2H, J=7.0 Hz), 4.32 (t, 2H, J=5.5 Hz), 3.85 (s, 2H), 2.70 (t, 2H, J=7.3 Hz), 2.58 (m, 2H), 1.59 (m, 2H), 0.87 (t, 3H, J=7.2 Hz).

MS: m/z=462.3 (M+H).

### EXAMPLE 33

### Preparation of 2-methyl-2-[1-(3-{[7-propyl-3-(trifluoromethyl)-1,2-benzisoxazol-6-yl]oxy}propyl)-1H-indol-5-yl]propanoic acid

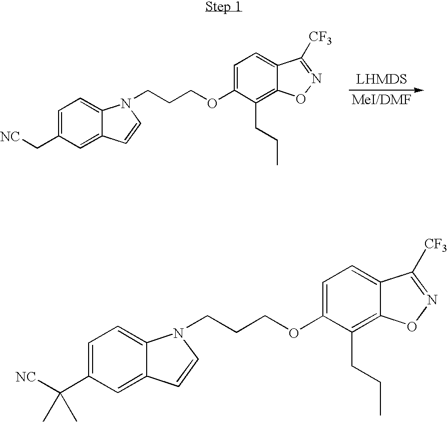

The nitrile (prepared according to the procedure of Example 31 Step 3, 0.05 g, 0.11 mmol) in DMF (0.5 mL) was cooled to −20° C. Lithium bis(trimethylsilyl)amide (1.0 M in hexanes, 1.0 ml, 0.136 mmol) was added and the reaction mixture stirred for 10 minutes. Methyl iodide (19 mg, 0.136) was added dropwise and the solution then allowed to warm to RT. The solution was re-cooled to −20° C., additional lithium bis(trimethylsilyl)amide (1.0 M in hexanes, 1.0 ml, 0.136 mmol) was added, and the reaction mixture stirred for 10 minutes. Methyl iodide (19 mg, 0.136) was added and the solution then allowed to warm to RT. The mixture was poured into saturated sodium bicarbonate followed by aqueous/ethyl acetate work-up and silica gel chromatography (ethyl acetate:hexanes (1:4)) to give the titled compound. 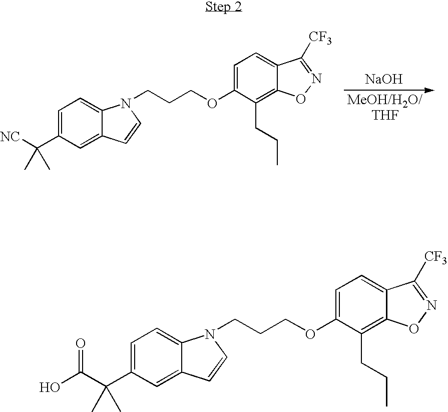
To the nitrile (prepared according to the procedure of Step 1, 31.0 mg, 0.066 mmol) in methanol (5 mL) was added sodium hydroxide (25% (wt., aq) (1.75 ml) and tetrahydrofuran (0.5 ml) and the resulting mixture stirred at 90° C. for 4 days. The reaction mixture was cooled to room temperature, poured into water, acidified with acetic acid, followed by aqueous/ethyl acetate work-up and RP-18 HPLC purification (acetonitrile:H₂O 15 minute gradient 10 to 100%:0.1% trifluoroacetic acid) to give the titled compound.
NMR (CD₃OD) δ7.56 (m, 2H), 7.3 (d, 1H, J=8.6 Hz), 7.15 (d, 1H, J=3.2 Hz), 7.13 (dd, 1H, J=6.8 Hz, J=1.8 Hz), 7.08 (d, 1H, J=8.9 Hz), 6.40 (d, 1H, J=3.1 Hz), 4.41 (t, 2H, J=6.7 Hz), 4.03 (t, 2H, J=5.8 Hz), 2.94 (t, 2H, J=7.4 Hz), 2.3 (m, 2H), 1.7 (m, 2H), 1.5 (s, 6H), 0.99 (t, 3H, J=7.4 Hz).
MS: m/z=489.4 (M+H).

### EXAMPLE 34

### Preparation of 2-[1-(3-{[7-propyl-3-(trifluoromethyl)-1,2-benzisoxazol-6-yl]oxy}propyl)-1H-indol-5-yl]propanoic acid

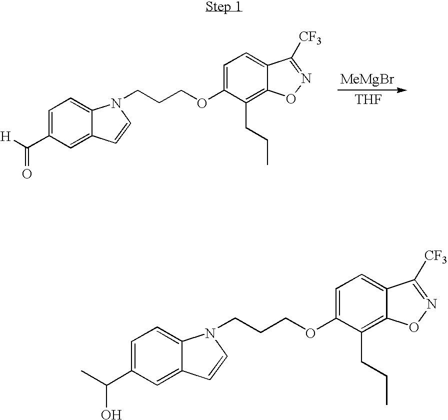 The aldehyde (prepared according to the procedure of Example 31 Step 1, 0.190 g, 0.442 mmol) was dissolved in tetrahydrofuran (2.25 ml) and cooled to −20° C. Methyl magnesium bromide (in THF) was added (3.0 M, 0.162 ml, 0.486 mmol) and the mixture stirred for 1 hour. The solution was poured into water followed by aqueous/ethyl acetate work-up and silica gel chromatography (ethyl acetate:hexanes (3:7)) to give the titled compound. 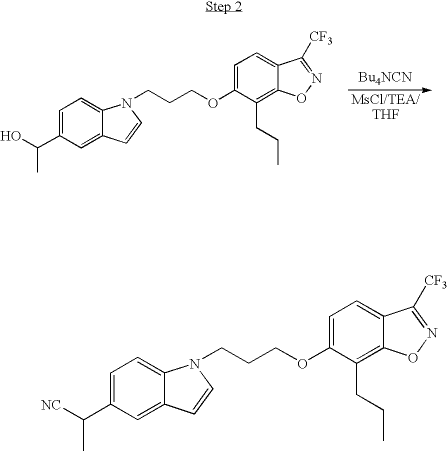
The alcohol (prepared according to the procedure of Step 1, 0.158 g, 0.35 mmol) in THF (1.77 mL) was cooled to −20° C. Triethylamine (0.11 g, 1.06 mmol) and methanesulfonyl chloride (0.05 g, 0.43 mmol) were added. The resulting solution was stirred for 10 minutes. Tetrabutylammonium cyanide (0.57 g, 2.1 mmol) was added into the reaction, the cooling bath was removed, and the reaction was stirred for 2.5 hours. The mixture was poured into saturated sodium bicarbonate followed by aqueous/ethyl acetate work-up and silica gel chromatography (ethyl acetate:hexanes (2:8)) to give the titled compound. 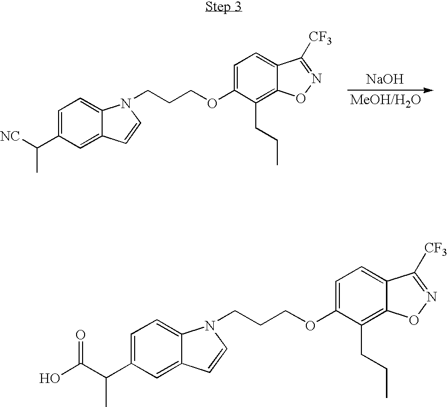
To the nitrile (prepared according to the procedure of Step 2, 29 mg, 0.064 mmol) in methanol (5 mL) and water (1 ml) was added sodium hydroxide (25% (wt., aq) (1.5 ml) and the resulting mixture was stirred at 105° C. 14 hours. The mixture was cooled to RT, poured into water, acidified with acetic acid, followed by aqueous/ethyl acetate work-up and silica gel chromatography (ethyl acetate:hexanes (3:7) 2% acetic acid) to give the titled compound.
NMR (CD₃OD) δ7.56 (d, 1H, J=8.8 Hz), 7.48 (s, 1H), 7.32 (d, 1H, J=8.5 Hz), 7.14 (d, 1H, J=3.2 Hz), 7.08 (m, 2H), 6.38 (d, 1H, J=3.0 Hz), 4.39 (t, 2H, J=6.7 Hz), 4.02 (t, 2H, J=5.7 Hz), 3.73 (q, 1H, J=7.2 Hz), 2.94 (t, 2H, J=7.4 Hz), 2.32 (m, 2H), 1.75 (m, 2H), 1.45 (d, 2H, J=7.1 Hz), 0.99 (t, 3H, J=7.4 Hz).
MS: m/z=475.4 (M+H).

### EXAMPLE 35

### Preparation of [1-(3-{[7-propyl-3-(trifluoromethyl)-1,2-benzisoxazol-6-yl]oxy}propyl)-1H-1,2,3-benzotriazol-5-yl]acetic acid

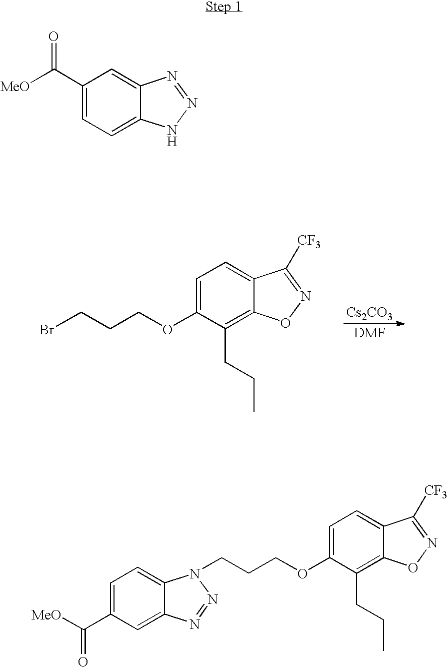 To a mixture of the bromide (prepared according to the procedure of Example 5, 0.30 g, 0.82 mmol), and methyl triazole-5-carboxyalate (0.16 g, 0.90 mmol) in DMF (2.3 mL) was added cesium carbonate (0.32 g, 0.98 mmol). The reaction mixture was stirred at room temperature overnight. Water was added followed by aqueous/ethyl acetate work-up and silica gel chromatography (ethyl acetate:hexanes (3:7)) to give the titled compound. 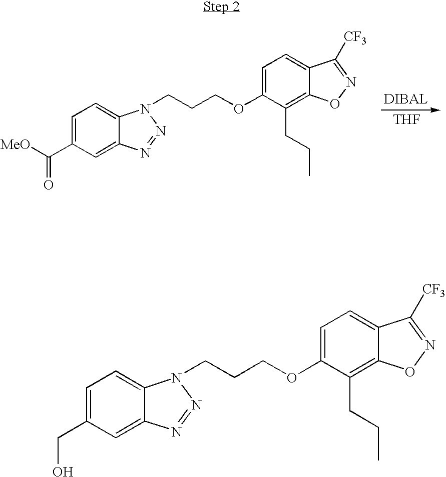
The ester (prepared according to the procedure of Step 1, 0.60 g, 0.13 mmol) was dissolved in THF (1.25 ml) and cooled to −20° C. Diisobutylaluminum hydride (1.0 M in hexanes, 0.288 ml, 0.288 mmol) was added dropwise and the mixture stirred for 1 hour being allowed to warm to room temperature. The reaction mixture was poured into 1 N sodium hydroxide followed by aqueous/ethyl acetate work-up to give the titled compound. 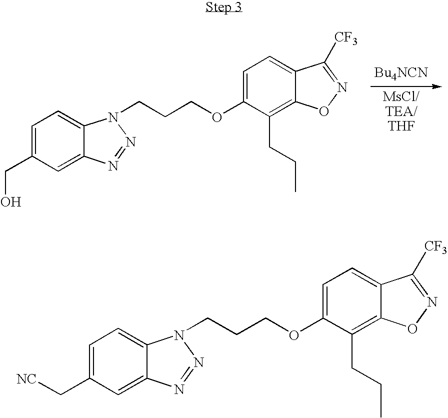
The alcohol (prepared according to the procedure of Step 2, 0.052 g, 0.12 mmol) in THF (0.6 mL) was cooled to −20° C. Triethylamine (0.036 g, 0.36 mmol) and methanesulfonyl chloride (0.016 g, 0.14 mmol) were added and the resulting solution stirred for 10 minutes. Tetrabutylamrmonium (0.19 g, 0.72 mmol) was added, the cooling bath removed, and the reaction was stirred 2.5 hours. The mixture was poured into 1 N sodium hydroxide followed by aqueous/dichloromethane work-up and silica gel chromatography (acetone:hexanes (4:6) 1.0% triethylamine) to give the titled compound.

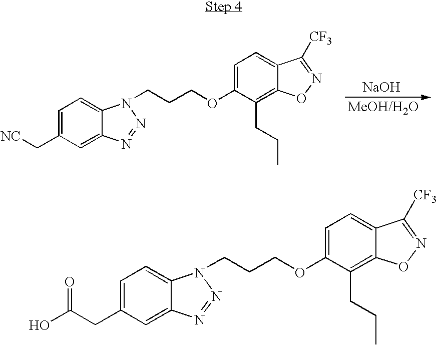

To the nitrile (prepared according to the procedure of Step 3, 20 mg, 0.045 mmol) in methanol (5 mL) was added sodium hydroxide (25% (wt., aq) (2.0 ml). The resulting mixture was stirred at reflux for 4 hours. The reaction mixture was allowed to cool to room temperature, poured into water, acidified with acetic acid, followed by aqueous/dichlormethane work-up, RP-18 HPLC purification (acetonitrile:H₂O 15 minute gradient 10 to 100%:0.1% trifluoroacetic acid) and silica gel chromatography (ethyl acetate:hexanes (1:1) 1.0% acetic acid) to give the titled compound.

NMR (CD₃OD) δ7.89 (s, 1H), 7.72 (d, 1H, J=8.5 Hz), 7.60 (d, 1H, J=8.8 Hz), 7.49 (d, 1H, J=8.5 Hz), 7.18 (d, 1H, J=8.9 Hz), 4.98 (t, 2H, J=6.6 Hz), 4.19 (t, 2H, J=5.6 Hz), 3.79 (s, 2H), 2.81 (t, 2H, J=7.4 Hz), 2.56 (m, 2H), 1.68 (m, 2H), 0.94 (t, 3H, J=7.4 Hz).

MS: m/z =463.4 (M+H).

### EXAMPLE 36

### Preparation of [2-oxo-1-(3-{[7-propyl-3-(trifluoromethyl)-1,2-benzisoxazol-6-yl]oxy}propyl)-2,3-dihydro-1H-indol-5-yl]acetic acid

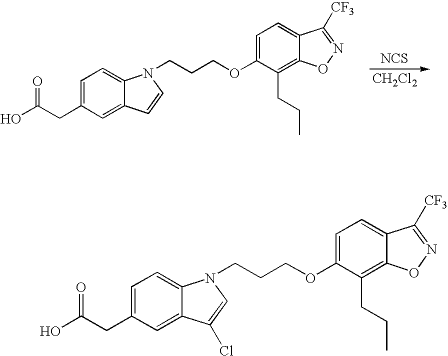 The acid (prepared according to the procedure of Example 30 Step 4, 0.104 g, 0.226 mmol), in dichloromethane (2.2 mL) was cooled to 0° C. N-chlorosuccinimide (0.033 g, 0.249 mmol) was added and the solution stirred for 6 hours being allowed to warm to room temperature. The reaction mixture was poured into water, acidified with acetic acid, followed by aqueous/ethyl acetate work-up, and silica gel chromatography (acetone:hexanes (4:6) 2.0% acetic acid) to give the titled compound.

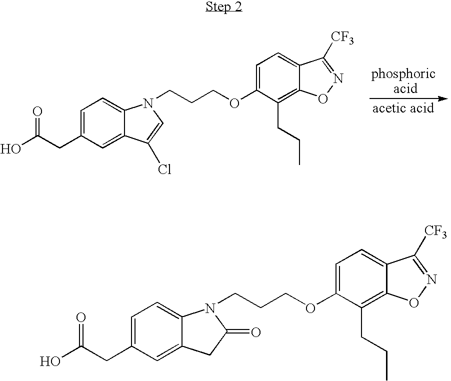

To the chloro-indole (prepared according to the procedure of Step 1, 0.318 g, 0.643 mmol) in acetic acid (4.0 mL) was added phosphoric acid (0.8 ml) and the mixture heated at reflux overnight. The reaction mixture was cooled to RT, poured into water, followed by aqueous/ethyl acetate work-up and silica gel chromatography (ethyl acetate:hexanes (4:6) 4.0% acetic acid) to give the titled compound.

NMR (CD₃OD) 67.62 (d, 1H, J=8.8 Hz), 7.24 (s, 1H), 7.23 (d, 1H, J=8.9 Hz), 7.18 (d, 1H, J=8.0 Hz), 6.97 (d, 1H, J=7.9 Hz), 4.22 (t, 2H, J=5.6 Hz), 4.00 (t, 2H, J=6.9 Hz), 3.56 (s, 2H), 3.53 (s, 2H), 2.90 (t, 2H, J=7.5 Hz), 2.23 (m, 2H), 1.71 (m, 2H), 0.95 (t, 3H, J=7.4 Hz).

MS: m/z=477.4 (M+H).

### EXAMPLE 37

### Preparation of [1-(3-{[7-propyl-3-(trifluoromethyl)-1,2-benzisoxazol-6-yl]oxy}propyl)-1H-indazol-5-yl]acetic acid

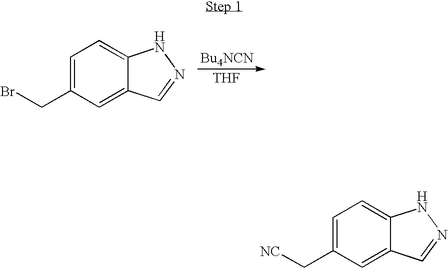 The bromide (prepared according to the procedure of J. Org. Chem. Soc. 1997, 62, 5627-5629, 0.050 g, 0.238 mmol) in THF (1.2 mL) was treated with tetrabutylammonium cyanide (0.383 g, 0.1.428 mmol) and the solution stirred for 0.5 hour at room temperature. The reaction mixture was poured into 1:1 saturated sodium bicarbonate:water, followed by aqueous/ethyl acetate work-up to give the titled compound. 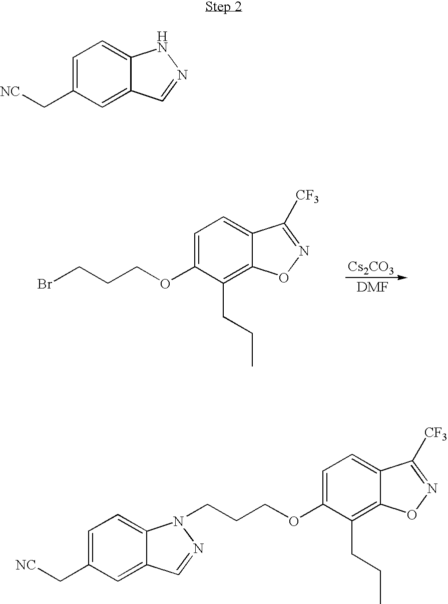
The bromide starting material was prepared according to the procedure of Example 5. The nitrile (prepared according to the procedure of Step 1, 0.022 g, 0.143 mmol) in DMF (0.7 mL) was treated with the bromide (0.46 g, 0.127 mmol) and cesium carbonate (0.136 g, 0.42 mmol). The resulting slurry was stirred for 1 hour. The reaction mixture was poured into water, followed by aqueous/ethyl acetate work-up and silica gel chromatography (ethyl acetate:hexanes (3:7) 4.0% acetic acid) to give the titled compound. 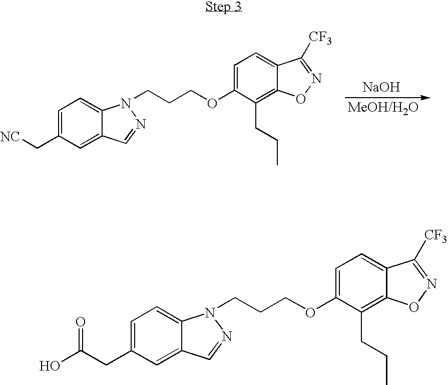
The nitrile (prepared according to the procedure of Step 2, 0.026 g, 0.59 mmol) in methanol (5.0 mL) was treated with sodium hydroxide (25% (aq) (wt), 2.0 ml). The resulting solution was refluxed for 1 hour, allowed to cool to room temperature, poured into water, acidified with acetic acid, followed by aqueous/ethyl acetate work-up, and RP-18 HPLC purification (acetonitrile: H₂O 15 minute gradient 10 to 100%:0.1% trifluoroacetic acid) to give the titled compound.
NMR (DMSO-d6) δ8.02 (s, 1H), 7.72 (d, 1H, J=8.7 Hz), 7.61 (s, 1H), 7.56 (d, 1H, J=8.7 Hz), 7.2 (m, 2H), 4.60 (t, 2H, J=6.7 Hz), 4.15 (t, 2H, J=5.8 Hz), 3.64 (s, 2H), 2.85 (t, 2H, J=7.4 Hz), 2.33 (m, 2H), 1.66 (m, 2H), 0.92 (t, 3H, J=7.4 Hz).
MS: m/z=462.4 (M+H).

### EXAMPLE 38

### Preparation of 6-chloro-1-(3-{[7-propyl-3-(trifluoromethyl)-1,2-benzisoxazol-6-yl]oxy}propyl)-1H-indole-5-carboxylic acid

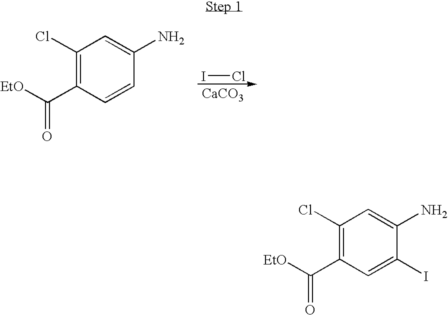 The indicated chloroaniline (1.02 g, 5.125 mmol) in methanol (13.0 mL) was treated with iodine monochloride (1.0 M in dichloromethane, 5.125 ml, 5.125 mmol) and the resulting solution stirred for 1 hour. The mixture was poured into water, followed by aqueous/ethyl acetate work-up, and silica gel chromatography (ethyl acetate:hexanes (2:8) 3.0% TEA) to give the titled compound. 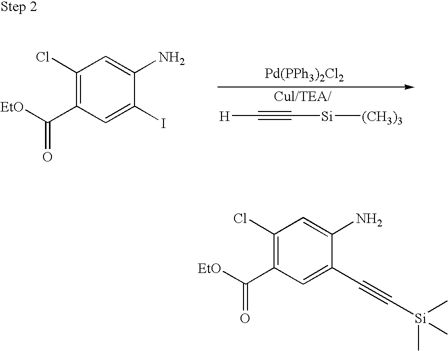
The iodide (prepared according to the procedure of Step 1, 125 mg, 0.384 mmol) was placed in an oven dried flask with copper iodide (7.0 mg, 0.038 nmrol), dichloro bis(triphenylphosphine)palladium(II) (27.0 mg, 0.038 mmol) and the solids slurried in triethylamine (2.0 mL). Trimethylsilylacetylene was added dropwise (41 mg, 0.422 mmol) and the slurry stirred at room temperature for 3 hours. Ethyl acetate (100 ml) was then added and the resulting solution washed with brine and dried over MgSO₄. The volatiles were removed in vacuo, and the residue was purified by silica gel chromatography (ethyl acetate:hexanes (2:8) 1.0% TEA) to give the titled compound. 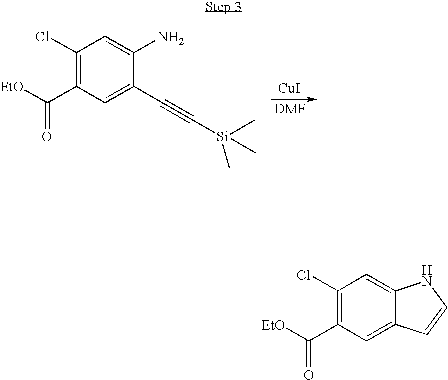
The alkyne (prepared according to the procedure of Step 2, 195 mg, 0.318 mmol) in DMF (1.3 mL) was treated with copper iodide (0.061 g, 0.318 mmol). The slurry was heated at 99° C. for 3 hours. The mixture was allowed to cool to room temperature, poured into water, acidified with acetic acid, followed by aqueous/ethyl acetate work-up, and silica gel chromatography (ethyl acetate:hexanes (2:8) 1.0% acetic acid) to give the titled compound. 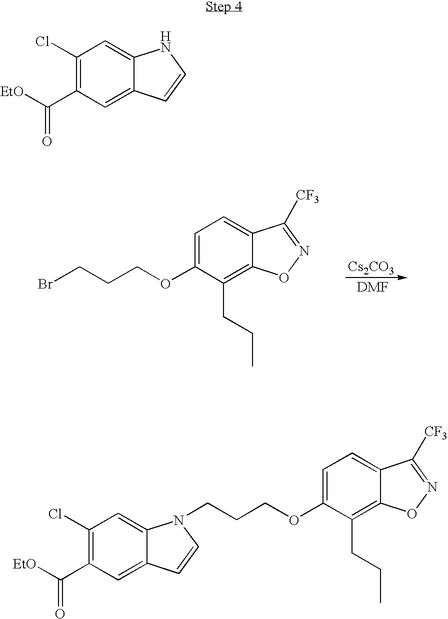
The bromide was prepared according to the procedure of Example 5. The indole (prepared according to the procedure of Step 3, 0.02 g, 0.094 mmol) in DMF (0.6 mL) was treated with the bromide (0.0.38 g, 0.103 mmol) and cesium carbonate (0.092 g, 0.282 mmol). The resulting slurry was stirred for 3 hours. The mixture was poured into water, followed by aqueous/ethyl acetate work-up and silica gel chromatography (ethyl acetate:hexanes (2:8)) to give the titled compound. 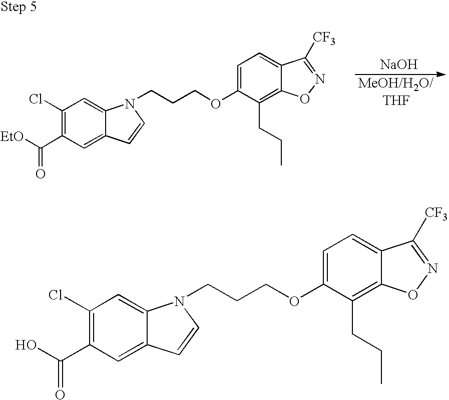
The ester (prepared according to the procedure of Step 4, 0.030 g, 0.59 mmol) in ethanol (1.0 mL) and THF (1.0 ml) was treated withl N sodium hydroxide (aq) (1.0 ml). The solution was heated at reflux for 2 hours. The reaction mixture was allowed to cool to room temperature, poured into water, acidified with acetic acid, followed by aqueous/ethyl acetate work-up, and silica gel chromatography (ethyl acetate:hexanes (2:8) 2.0% acetic acid) to give the titled compound.
NMR (CD₃OD) δ8.16 (s, 1H), 7.59 (d, 1H, J=8.8 Hz), 7.48 (s, 1H), 7.34 (d, 1H, J=3.2 Hz), 7.13 (d, 1H, J=8.9 Hz), 6.57 (d, 1H, J=3.1 Hz), 4.45 (t, 2H, J=6.7 Hz), 4.08 (t, 2H, J=5.6 Hz), 2.97 (t, 2H, J=7.4 Hz), 2.36 (m, 2H), 1.77 (m, 2H), 1.00 (t, 3H, J=7.0 Hz).
MS: m/z=481 (M+H).

### EXAMPLE 39

### Preparation of 4-chloro-1-(3-{[7-propyl-3-(trifluoromethyl)-1,2-benzisoxazol-6-yl]oxy}propyl)-1H-indole-5-carboxylic acid

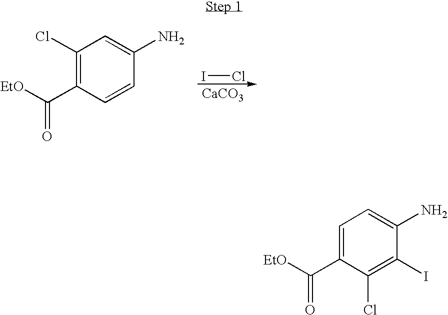 The indicated chloro-aniline (1.02 g, 5.125 mmol) in methanol (13.0 mL) was treated with iodine monochloride (1.0 M in dichloromethane, 5.125 ml, 5.125 mmol) and the solution stirred for 1 hour at room temperature. The reaction mixture was poured into water, followed by aqueous/ethyl acetate work-up, and silica gel chromatography (ethyl acetate:hexanes (2:8) 3.0% TEA) to give the titled compound. 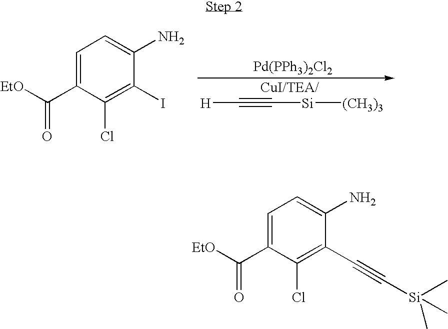
The iodide (prepared according to the procedure of Step 1, 636 mg, 1.95 mmol) was placed in an oven dried flask with copper iodide (37.0 mg, 0.195 mmol) and dichloro bis(triphenylphosphine)palladium(II) (137 mg, 0.195 mmol) and the solids slurried in triethylamine (10.2 mL). Trimethylsilylacetylene was added dropwise at 0° C. (211 mg, 2.419 mmol) and the slurry stirred at room temperature overnight. Then ethyl acetate (100 ml) was added and the resulting solution washed with brine and dried over MgSO₄. The volatiles were removed in vacuo followed by purification of the residue by silica gel chromatography (ethyl acetate:hexanes (3:7) 1.0% TEA) to give the titled compound. 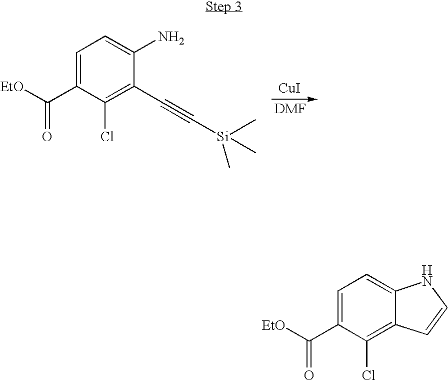
The alkyne (prepared according to the procedure of Step 2, 280 mg, 0.946 mmol) in DMF (3.8 mL) was treated with copper iodide (0.36 g, 0.1.89 mmol) and the resulting slurry stirred for 4.0 hours at 99° C. The reaction mixture was allowed to cool to room temperature, poured into water, acidified with acetic acid, followed by aqueous/ethyl acetate work-up, and silica gel chromatography (ethyl acetate:hexanes (3:7) 1.0% acetic acid) to give the titled compound. 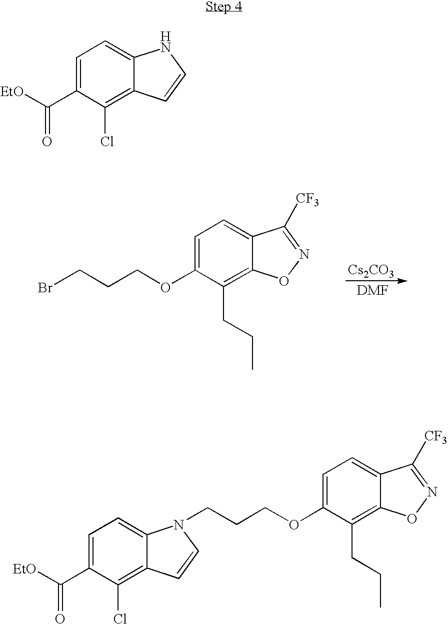
The bromide was prepared according to the procedure of Example 5. The indole (prepared according to the procedure of Step 3, 0.078 g, 0.35 mmol) in DMF (1.75 mL) was treated with the bromide (0.141 g, 0.385 mmol) and cesium carbonate (0.341 g, 1.049 mmol). The resulting slurry was stirred overnight, then poured into water, followed by aqueous/ethyl acetate work-up and silica gel chromatography (acetone:hexanes (2:8)) to give the titled compound. 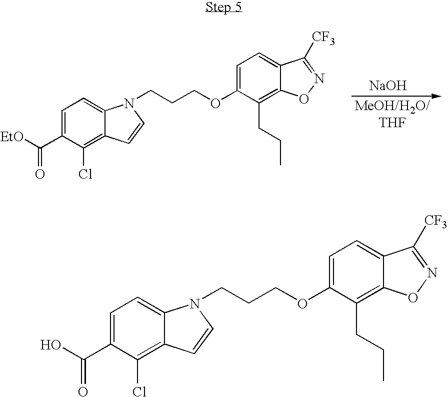
The ester (prepared according to the procedure of Step 4, 0.135 g, 0.67 mmol) in ethanol (1.0 mL) and THF (1.2 ml) was treated with 1 N sodium hydroxide (aq) (1.0 ml). The solution was heated to reflux for 2 hours, allowed to cool to RT, poured into water, acidified with acetic acid, followed by aqueous/ethyl acetate work-up and silica gel chromatography (ethyl acetate:hexanes (3:7) 2.0% acetic acid) to give the titled compound.
NMR (CD₃OD) δ7.69 (d, 1H, J=8.7 Hz), 7.59 (d, 1H, J=8.6 Hz), 7.41 (d, 1H, J=8.7 Hz), 7.37 (d, 1H, J=3.2 Hz), 7.14 (d, 1H, J=8.9 Hz), 6.69 (d, 1H, J=3.2 Hz), 4.48 (t, 2H, J=6.8 Hz), 4.10 (t, 2H, J=5.7 Hz), 2.91 (t, 2H, J=7.3 Hz), 2.3 (m, 2H), 1.7 (m, 2H), 0.98 (t, 3H, J=7.3 Hz).
MS: m/z=480.9 (M+H).

### EXAMPLE 40

### Preparation of [6-chloro-1-(3-{[7-propyl-3-(trifluoromethyl)-1,2-benzisoxazol-6-yl]oxy}propyl)-1H-indol-5-yl]acetic acid

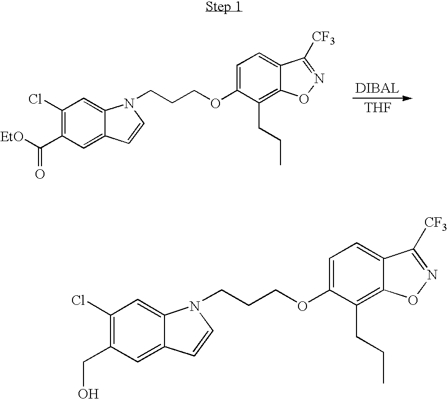 The ester (prepared according to the procedure of Example 38 Step 4, 0.363 g, 0.713 mmol) was dissolved in THF (7.1 ml) and cooled to −20° C. Diisobutylaluminum hydride (1.0 M in hexanes, 1.5 ml, 1.5 mmol) was added and the reaction mixture stirred for 1 hour. The reaction was allowed to warm to room temperature, ethyl acetate was added (12 ml) followed by addition of 0.54 g celite and saturated aqueous ammonium chloride (0.64 ml). The slurry was filtered and the resulting solids washed with ethyl acetate. The organics were combined and washed with water, brine, and dried over MgSO₄. The volatiles were removed in vacuo followed by silica gel chromatography (ethyl acetate:hexanes (3:7) 1.0% acetic acid) to give the titled compound. 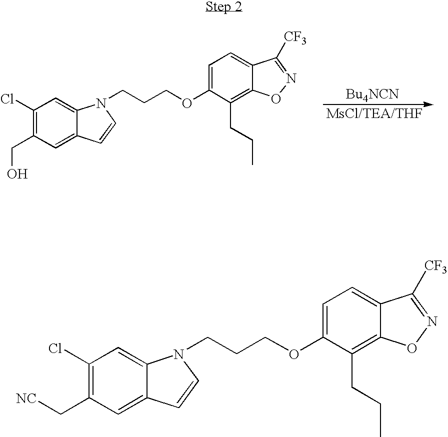
The alcohol (prepared according to the procedure of Step 1, 0.23 g, 0.49 mmol) was dissolved in THF (2.5 mL). Triethylamine was added (0.149 g, 1.5 mmol) and the solution was cooled to −20° C. Methanesulfonyl chloride (0.016 g, 0.14 mmol) was added and the solution stirred for 10 minutes. Tetrabutylammonium cyanide (0.19 g, 0.70 mmol) was added, the cooling bath was removed, and the reaction was stirred for 1 hour. The mixture was poured into water: saturated sodium bicarbonate (1:4) followed by aqueous/ethyl acetate work-up and silica gel chromatography (ethyl acetate:hexanes (3:7) 1.0% acetic acid) to give the titled compound. 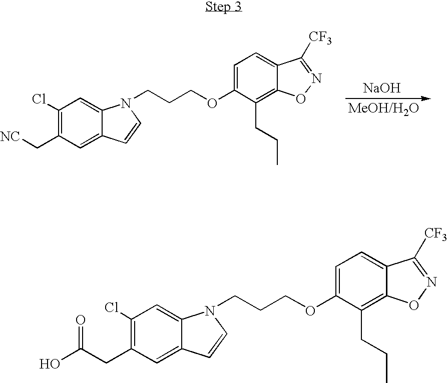
The nitrile (prepared according to the procedure of Step 2, 210.0 mg, 0.441 mmol) was dissolved in methanol (9.0 mL) and THF (4.0 ml). Sodium hydroxide (50% wt. aq 2.5 ml) was added and the solution stirred at reflux for 9 hours. The reaction mixture was allowed to cool to room temperature, poured into water, acidified with acetic acid, followed by aqueous/ethyl acetate work-up and silica gel chromatography (ethyl acetate:hexanes (4:6) 3.0% acetic acid) to give the titled compound.
NMR (CD₃OD) δ7.59 (d, 1H, J=9.0 Hz), 7.49 (s, 1H), 7.42 (s, 1H), 7.22 (d, 1H, J=3.2 Hz), 7.11 (d, 1H, J=8.9 Hz), 6.43 (d, 1H, J=3.5 Hz), 4.40 (t, 2H, J=6.6 Hz), 4.06 (t, 2H, J=5.7 Hz), 3.76 (s, 2H), 2.99 (t, 2H, J=7.6 Hz), 2.34 (m, 2H), 1.78 (m, 2H), 1.01 (t, 3H, J=7.3 Hz).
MS: m/z=494.9 (M+H).

### EXAMPLE 41

### Preparation of [6-chloro-2-oxo-1-(3-{[7-propyl-3-(trifluoromethyl)-1,2-benzisoxazol-6-yl]oxy}propyl)-2,3-dihydro-1H-indol-5-yl]acetic acid

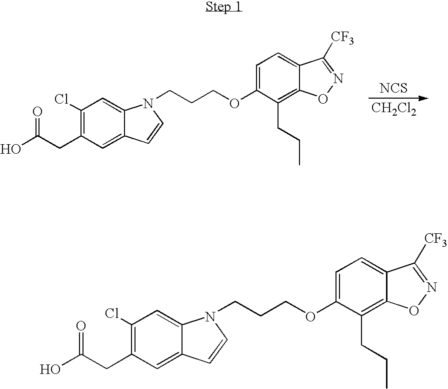 The acid (prepared according to the procedure of Example 40 Step 3, 0.065 g, 0.127 mmol) in dichloromethane (1.3 mL) was cooled to 0° C. N-chlorosuccinimide (0.017 g, 0.127 mmol) was added and the solution allowed to warm to room temperature over 4 hours. The mixture was poured into water, acidified with acetic acid, followed by aqueous/ethyl acetate work-up, and silica gel chromatography (acetone:hexanes (3:7) 3.0% acetic acid) to give the titled compound. 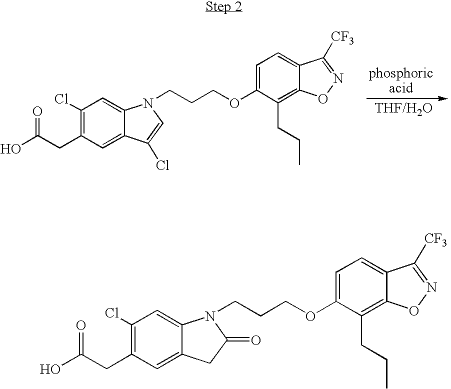
The chloro-indole (prepared according to the procedure of Step 1, 0.031 g, 0.059 mmol) in acetic acid (1.0 mL) was treated with phosphoric acid (2.0 ml) and stirred at 95° C. for 2 hours. The reaction mixture was allowed to cool to room temperature, poured into water, followed by aqueous/ethyl acetate work-up and RP-18 HPLC purification (acetonitrile: H₂O 15 minute gradient 30 to 100%:0.1% trifluoroacetic acid) to give the titled compound.
NMR (acetone-d6) δ7.60 (d, 1H, J=8.9 Hz), 7.22 (m, 2H), 7.00 (s, 1H), 4.22 (t, 2H, J=6.0 Hz), 3.94 (t, 2H, J=6.8 Hz), 3.65 (s, 2H), 3.48 (s, 2H), 2.89 (t, 2H, J=7.2 Hz), 2.18 (m, 2H), 1.69 (m, 2H), 0.92 (t, 3H, J=7.3 Hz).
MS: m/z=510.9 (M+H).

### EXAMPLE 42

### Preparation of [4-chloro-1-(3-{[7-propyl-3-(trifluoromethyl)-1,2-benzisoxazol-6-yl]oxy}propyl)-1H-indol-5-yl]acetic acid

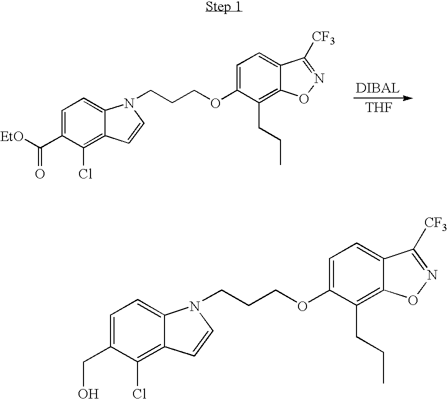 The ester (prepared according to the procedure of Example 39 Step 4, 0.062 g, 0.12 mmol) was dissolved in THF (1.2 ml) and cooled to −20° C. Diisobutylaluminum hydride (1.0 M in hexanes, 0.29 ml, 0.27 mmol) was added and the solution stirred for 1 hour. The reaction was allowed to warm to room temperature, poured into water, acidified with acetic acid, followed by aqueous/ethyl acetate work-up and silica gel chromatography (ethyl acetate:hexanes (3:7) 2.0% acetic acid) to give the titled compound. 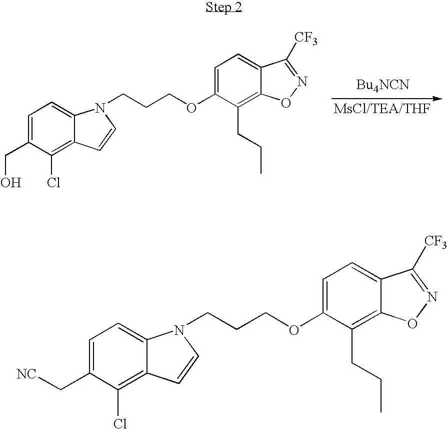
The alcohol (prepared according to the procedure of Step 1, 0.042 g, 0.090 mmol) in THF (0.45 mL) was cooled to −20° C. Triethylamine and (0.027 g, 0.27 mmol) methanesulfonyl chloride (0.012 g, 0.11 mmol) were added and the solution stirred for 10 minutes. Tetrabutylammonium cyanide (0.15 g, 0.54 mmol) was added, the cooling bath removed, and the reaction stirred for 1 hour. The mixture was poured into water:saturated sodium bicarbonate (1:1) followed by aqueous/ethyl acetate work-up and silica gel chromatography (ethyl acetate:hexanes (3:7) 1.0% acetic acid) to give the titled compound. 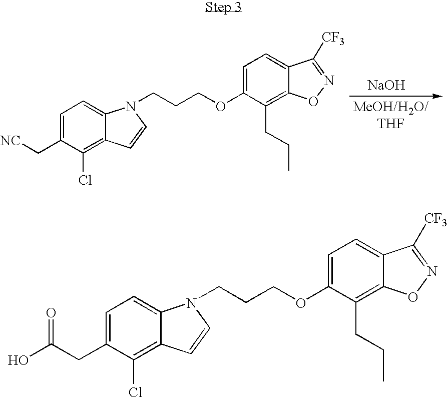
The nitrile (prepared according to the procedure of Step 2, 37.0 mg, 0.441 mmol) was dissolved in methanol (1.8 mL) and THF (0.8 ml). Sodium hydroxide (50% wt., aq 0.5 ml) was added and the resulting solution stirred at 100° C. for 5 hours. The reaction mixture was allowed to cool to room temperature, poured into water, acidified with acetic acid, followed by aqueous/ethyl acetate work-up and silica gel chromatography (ethyl acetate:hexanes (3:7) 3.0% acetic acid) to give the titled compound.
NMR (CD₃OD) δ7.59 (d, 1H, J=8.7 Hz), 7.33 (d, 1H, J=8.2 Hz), 7.26 (d, 1H, J=3.1 Hz), 7.13 (d, 1H, J=8.8 Hz), 7.07 (d, 1H, J=8.4 Hz), 6.52 (d, 1H, J=3.0 Hz), 4.44 (t, 2H, J=6.7 Hz), 4.07 (t, 2H, J=5.8 Hz), 3.82 (s, 2H), 2.95 (t, 2H, J=7.4 Hz), 2.36 (m, 2H), 1.75 (m, 2H), 0.99 (t, 3H, J=7.5 Hz).
MS: m/z=494.9 (M+H).

### EXAMPLE 43

### Preparation of 6-methoxy-1-(3-{[7-propyl-3-(trifluoromethyl)-1,2-benzisoxazol-6-yl]oxy}propyl)-1H-indole-5-carboxylic acid

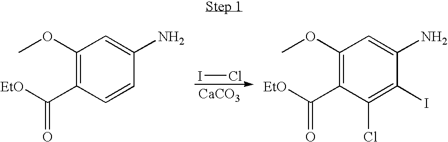 The indicated aniline (3.0 g, 16.56 mmol) in methanol (45.0 mL) was treated with 1.0 M iodine monochloride (1M in dichloromethane, 17.1 ml, 17.1 mmol) and the solution stirred for 1 hour at room temperature. The reaction mixture was poured into water, followed by aqueous/ethyl acetate work-up, and silica gel chromatography (ethyl acetate:hexanes (4:6)) to give the titled compound. 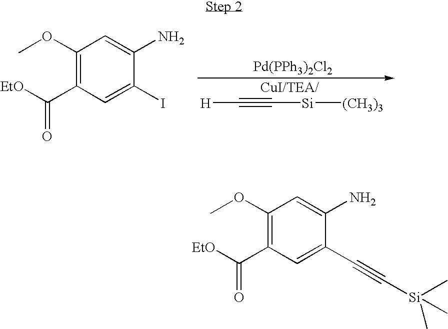
The indicated iodide (prepared according to the procedure of Step 1, 368 mg, 1.2 mmol) was placed in an oven dried flask with copper iodide (46.0 mg, 0.24 mmol) and dichloro bis(triphenylphosphine)palladium(II) (168.0 mg, 0.24 mmol) and the solids slurried in triethylamine (6.0 mL). Trimethylsilylacetylene was added dropwise (130 mg, 1.32 mmol) and the slurry stirred at room temperature for 3.5 hours. Then ethyl acetate (400 ml) was added and the resulting solution washed with brine and dried over MgSO₄. The volatiles were removed in vacuo, followed by purification of the residue by silica gel chromatography (ethyl acetate:hexanes (4:6) 2.0% TEA) to give the titled compound. 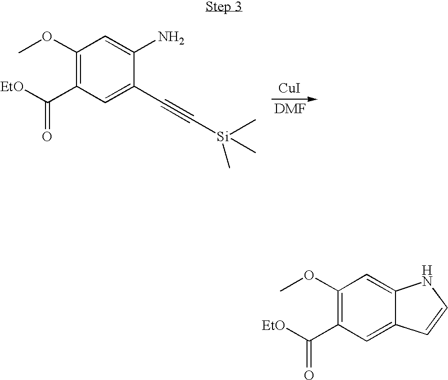
The alkyne (prepared according to the procedure of Step 2, 231 mg, 0.83 mmol) in DMF (3.3 mL) was treated with copper iodide (0.317 g, 1.666 mmol) and stirred for 3.0 hours at 99° C. The reaction mixture was cooled to RT, poured into water, acidified with acetic acid, followed by aqueous/ethyl acetate work-up, and silica gel chromatography (ethyl acetate:hexanes (1:1) 3.0% acetic acid) to give the titled compound. 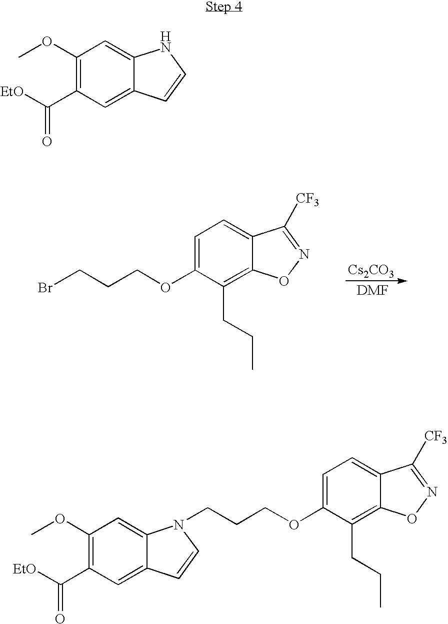
The bromide starting material was prepared according to the procedure of Example 5. The indole (prepared according to the procedure of Step 3, 0.060 g, 0.292 mmol) in DMF (2.0 mL) was treated with the bromide (0.118 g, 0.321 mmol) and cesium carbonate (0.285 g, 0.877 mmol) and the resulting slurry stirred for 3 hours. The reaction mixture was poured into water, followed by aqueous/ethyl acetate work-up and silica gel chromatography (ethyl acetate:hexanes (4:6)) to give the titled compound. 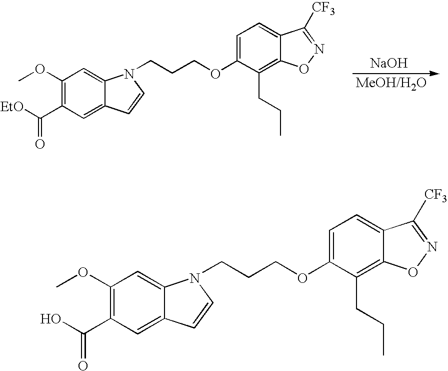
The ester (prepared according to the procedure of Step 4, 0.030 g, 0.061 mmol) in methanol (2.0 mL) was treated with 1 N sodium hydroxide (aq, 0.25 ml). The resulting solution was heated at reflux for 2 hours, allowed to cool to room temperature and stirred overnight. The reaction mixture was poured into water, acidified with acetic acid, followed by aqueous/ethyl acetate work-up, and silica gel chromatography (ethyl acetate:hexanes (4:6) 3.0% acetic acid) to give the titled compound.
NMR(CD₃OD)88.17 (s, 1H), 7.59 (d, 1H, J=8.6 Hz), 7.20 (d, 1H, J=3.3 Hz), 7.13 (d, 1H, J=8.8 Hz), 7.00 (s, 1H), 6.50 (d, 1H, J=3.2 Hz), 4.43 (t, 2H, J=6.6 Hz), 4.09 (t, 2H, J=5.7 Hz), 3.79 (s, 3H), 2.94 (t, 2H, J=7.4 Hz), 2.37 (m, 2H), 1.75 (m, 2H), 0.98 (t, 3H, J=7.4 Hz).
MS: m/z=476.9 (M+H).

### EXAMPLE 44

### Preparation of 3-[1-(3-{[7-propyl-3-(trifluoromethyl)-1,2-benzisoxazol-6-yl]oxy}propyl)-1H-indol-5-yl]propanoic acid

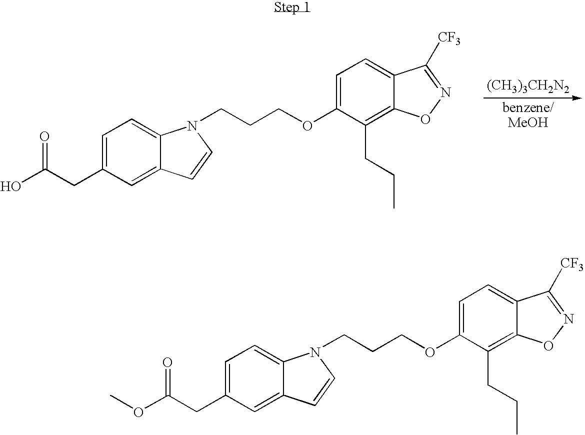 The acid (prepared according to the procedure of Example 30 Step 4, 1.58 g, 3.34 mmol) in benzene (26.4 ml) and methanol (6.6 ml) was treated with trimethylsilyldiazomethane (2.0 M in hexanes, 2.17 ml, 4.34 mmol) and the solution stirred for 1 hour. The mixture was poured into saturated aqueous sodium bicarbonate, followed by aqueous/ethyl acetate work-up to give the titled compound.

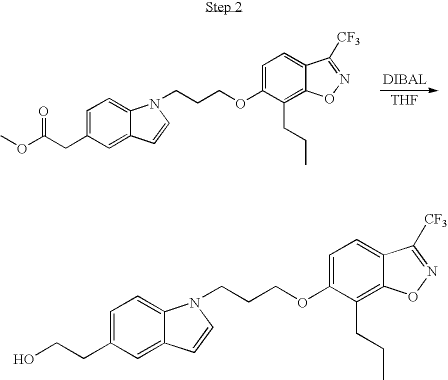

The ester (prepared according to the procedure of Step 1, 0.150 g, 0.316 mmol) was dissolved in THF (3.0 ml) and cooled to −20° C. Diisobutylaluminum hydride (1.0 M in hexanes, 0.695 ml, 695 mmol) was added, and the mixture stirred for 1 hour. The solution was allowed to warm to room temperature, ethyl acetate was added (6.0 ml) followed by addition of 0.54 g celite and saturated aqueous ammonium chloride (0.35 ml). The slurry was stirred 10 minutes, filtered, and the resulting solids washed with ethyl acetate. The organics were combined, washed with water, brine, and dried over MgSO₄. The volatiles were removed in vacuo followed by purification of the residue by silica gel chromatography (ethyl acetate:hexanes (3:7) 3.0% acetic acid) to give the titled compound. 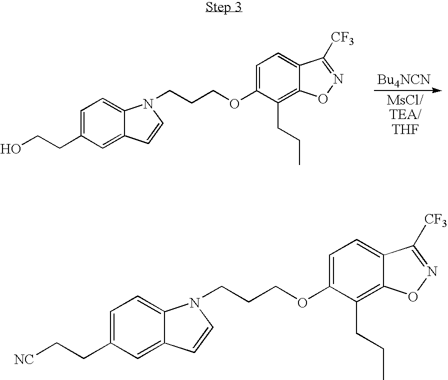
The alcohol (prepared according to the procedure of Step 2, 0.093 g, 0.21 mmol) in THF (1.0 mL) was treated with triethylamine (0.063 g, 0.624 mmol) and the solution was cooled to −20° C. Methanesulfonyl chloride (0.029 g, 0.25 mmol) was added and the solution stirred for 10 minutes. Tetrabutylammonium cyanide (0.335 g, 1.25 mrol) was added, the cooling bath was removed, and the reaction was stirred for 1 hour. The mixture was poured into water:saturated sodium bicarbonate (1:4) followed by aqueous/ethyl acetate work-up and silica gel chromatography (ethyl acetate:hexanes (1:3) 1.0% acetic acid) to give the titled compound. 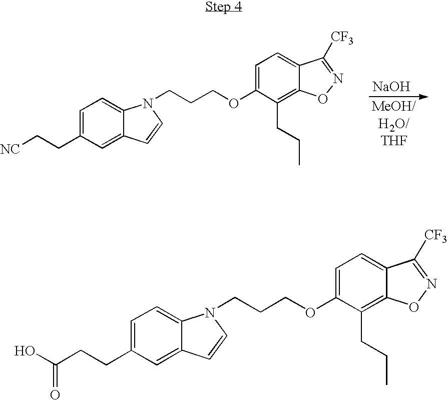
The nitrile (prepared according to the procedure of Step 3, 40.0 mg, 0.089 mmol) in methanol (1.8 mL) and THF (0.8 mil) was treated with sodium hydroxide (50% wt., aq, 0.5 ml) and heated at reflux for 3 hours. The reaction mixture was allowed to cool to room temperature, poured into water, acidified with acetic acid, followed by aqueous/ethyl acetate work-up and silica gel chromatography (ethyl acetate:hexanes (3:7) 3.0% acetic acid) to give the titled compound.
NMR (CD₃OD) δ7.58 (d, 1H, J=8.9 Hz), 7.38 (s, 1H), 7.30 (d, 1H, J=8.5 Hz), 7.14 (d, 1H, J=3.0 Hz), 7.11 (d, 1H, J=8.9 Hz), 6.98 (d, 1H, J=8.4 Hz), 6.36 (d, 1H, J=2.8 Hz), 4.40 (t, 2H, J=6.9 Hz), 4.05 (t, 2H, J=5.7 Hz), 2.96 (t, 2H, J=7.3 Hz), 2.59 (t, 2H, J=8.0 Hz), 2.34 (m, 2H), 1.76 (m, 2H), 1.00 (t, 3H, J=7.6 Hz).
MS: m/z=474.9 (M+H).

The foregoing examples are provided to illustrate the invention and should not be construed as limiting the scope of the invention in any way.

What is claimed is:

1. A compound having Formula I: 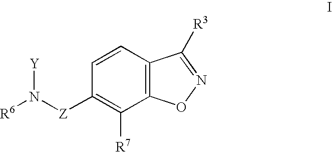
or a pharmaceutically acceptable salt thereof, wherein
Y is selected from the group consisting of: 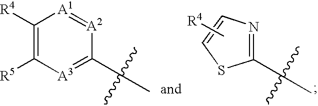
A¹, A², and A³ are each independently selected from the group consisting of —C(R¹)═ and —N═, wherein one or both of A¹ and A² is —C(R¹)═;
R¹ is selected from the group consisting of —H, halogen, —C₁-C₆alkyl and —OC₁-C₆alkyl, wherein —C₁-C₆alkyl and —OC₁-C₆alkyl are each optionally substituted with 1-3 halogens;
R⁶ is C₁-C₄alkyl; or Y and R⁶ and the N to which they are attached taken together form a bicyclic heterocyclic ring selected from the group consisting of: 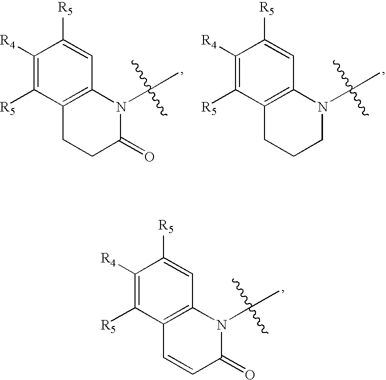 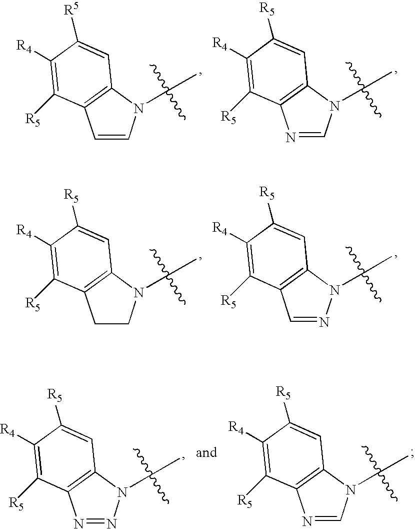
Z is a difunctional connecting moiety selected from the group consisting of C₁-C₄alkylene and —C₁-C₃alkyleneO— , wherein the O of —C₁-C₃alkyleneO— is connected to the benzisoxazole ring in Formula I;
R³ is —C₁-C₆ alkyl, which is optionally substituted with 1-3 halogens;
R⁴ is selected from the group consisting of —CO₂R² and —C₁-C₄alkylCO₂R², with the proviso that when A¹, A² and A³ are all equal to —C(R¹)═ and R⁶ is C₁-C₄alkyl, then R⁴ is —CO₂R²;
R² is selected from the group consisting of H and C₁-C₄alkyl;
R⁵ is selected from the group consisting of H, halogen, C₁-C₅alkyl, and —OC₁-C₅alkyl; and
R⁷ is —C₁-C₆ alkyl, which is optionally substituted with 1-3 halogens.

2. A compound of claim 1, or a pharmaceutically acceptable salt thereof, wherein
Y is selected from the group consisting of: 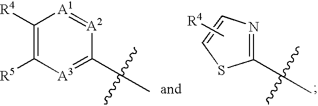
A¹, A², and A³ are each independently selected from the group consisting of —C(R¹)═ and —N═, wherein one or both of A¹ and A² is —C(R¹)═;
R¹ is selected from the group consisting of —H, halogen, —CH₃, —CF₃, —OCH₃, and —OCF₃;
R⁶ is C₁-C₄alkyl;
Z is —C₁-C₃alkyleneO— , wherein the O of —C₁-C₃alkyleneO— is connected to the benzisoxazole ring in Formula I; and
R⁵ is selected from the group consisting of H, halogen, —CH₃, and —OCH₃.

3. A compound of claim 2, or a pharmaceutically acceptable salt thereof, wherein
R⁶ is CH₃;
Z is —CH₂CH₂CH₂O—, wherein the —O— of —CH₂CH₂CH₂O— is connected to the benzisoxazole ring in Formula I;
R³ is —CF₃;
R⁴ is selected from the group consisting of —CO₂H and —C₁-C₄alkylCO₂H, with the proviso that when A¹, A² and A³ are all equal to —C(R¹)═, then R⁴ is —CO₂H; and
R⁷ is —CH₂CH₂CH₃.

4. A compound of claim 1, or a pharmaceutically acceptable salt thereof, wherein
Y and R⁶ and the N to which they are attached taken together form a bicyclic heterocyclic ring selected from the group consisting of: 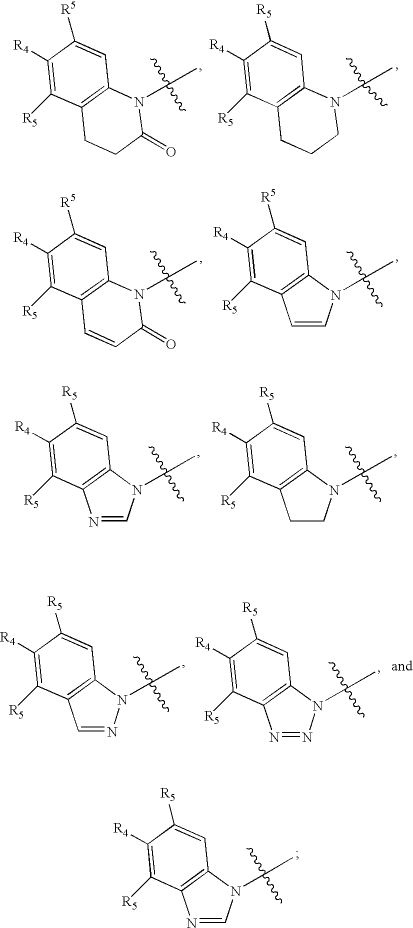
Z is a difunctional connecting moiety selected from the group consisting of C₁-C₄alkylene and —C₁-C₃alkyleneO— , wherein the O of —C₁-C₃alkyleneO— is connected to the benzisoxazole ring in Formula I;
R⁴ is selected from the group consisting of —CO₂R² and —C₁-C₄alkylCO₂R²; and
R⁵ is selected from the group consisting of H, halogen, —CH₃, and —OCH₃.

5. A compound of claim 4, or a pharmaceutically acceptable salt thereof, wherein
Z is —CH₂CH₂CH₂O—, wherein the —O— of —CH₂CH₂CH₂O— is connected to the benzisoxazole ring in Formula I;
R³ is —CF₃;
R⁴ is selected from the group consisting of —CO₂H and —C₁-C₄alkylCO₂H; and
R⁷ is —CH₂CH₂CH₃.

6. A compound of claim 1, selected from the group consisting of:
(1) 1-(3-{[7-propyl-3-(trifluoromethyl)-1,2-benzisoxazol-6-yl]oxy}propyl)-1H-indole-5-carboxylic acid,
(2) 4-[methyl(3-{[7-propyl-3-(trifluoromethyl)-1,2-benzisoxazol-6-yl]oxy}propyl)amino]benzoic acid,
(3) 3-methyl-4-[methyl(3-{[7-propyl-3-(trifluoromethyl)-1,2-benzisoxazol-6-yl]oxy}propyl)amino]benzoic acid,
(4) [2-oxo-1-(3-{[7-propyl-3-(trifluoromethyl)-1,2-benzisoxazol-6-yl]oxy}propyl)-1,2,3,4-tetrahydroquinolin-6-yl]acetic acid,
(5) 1-(3-{[7-propyl-3-(trifluoromethyl)-1,2-benzisoxazol-6-yl]oxy}propyl)-1,2,3,4-tetrahydroquinoline-6-carboxylic acid,
(6) [1-(3-{[7-propyl-3-(trifluoromethyl)-1,2-benzisoxazol-6-yl]oxy}propyl)-1,2,3,4-tetrahydroquinolin-6-yl]acetic acid,
(7) 3-chloro-4-[methyl(3-{[7-propyl-3-(trifluoromethyl)-1,2-benzisoxazol-6-yl]oxy}propyl)amino]benzoic acid,
(8) 2-chloro-4-[methyl(3-{[7-propyl-3-(trifluoromethyl)-1,2-benzisoxazol-6-yl]oxy}propyl)amino]benzoic acid,
(9) 4-[methyl(3-{[7-propyl-3-(trifluoromethyl)-1,2-benzisoxazol-6-yl]oxy}propyl)amino]-3-(trifluoromethoxy)benzoic acid,
(10) [2-oxo-1-(3-{[7-propyl-3-(trifluoromethyl)-1,2-benzisoxazol-6-yl}oxy) propyl)-1,2-dihydroquinolin-6-yl]acetic acid,
(11) 2-oxo-1-(3-{[7-propyl-3-(trifluoromethyl)-1,2-benzisoxazol-6-yl]oxy}propyl)indoline-5-carboxylic acid,
(12) 2-methoxy-4-[methyl(3-{[7-propyl-3-(trifluoromethyl)-1,2-benzisoxazol-6-yl]oxy}propyl)amino]benzoic acid,
(13) 4-[methyl(3-{[7-propyl-3-(trifluoromethyl)-1,2-benzisoxazol-6-yl]oxy}propyl)amino]-2-(trifluoromethyl)benzoic acid,
(14) 2-fluoro-4-[methyl(3-{[7-propyl-3-(trifluoromethyl)-1,2-benzisoxazol-6-yl]oxy}propyl)amino]benzoic acid,
(15) 2-bromo-4-[methyl(3-{[7-propyl-3-(trifluoromethyl)-1,2-benzisoxazol-6-yl]oxy}propyl)amino]benzoic acid,
(16) 6-[methyl(3-{[7-propyl-3-(trifluoromethyl)-1,2-benzisoxazol-6-yl]oxy}propyl)amino]nicotinic acid,
(17) {6-[methyl(3-{[7-propyl-3-(trifluoromethyl)-1,2-benzisoxazol-6-yl]oxy}propyl)amino]pyridin-3-yl}acetic acid,
(18) 5-[methyl(3-{[7-propyl-3-(trifluoromethyl)-1,2-benzisoxazol-6-yl]oxy}propyl)amino]pyrazine-2-carboxylic acid,
(19) {5-[methyl(3-{[7-propyl-3-(trifluoromethyl)-1,2-benzisoxazol-6-yl]oxy}propyl)amino]pyrazin-2-yl}acetic acid,
(20) 2-[methyl(3-{[7-propyl-3-(trifluoromethyl)-1,2-benzisoxazol-6-yl]oxy}propyl)amino]pyrimidine-5-carboxylic acid,
(21) {2-[methyl(3-{[7-propyl-3-(trifluoromethyl)-1,2-benzisoxazol-6-yl]oxy}propyl)amino]pyrimidin-5-yl}acetic acid,
(22) {2-[methyl(3-{[7-propyl-3-(trifluoromethyl)-1,2-benzisoxazol-6-yl]oxy}propyl)amino]-1,3-thiazol-4-yl}acetic acid,
(23) 2-[methyl(3-{[7-propyl-3-(trifluoromethyl)-1,2-benzisoxazol-6-yl]oxy}propyl)amino]-1,3-thiazole-5-carboxylic acid,
(24) {2-[methyl(3-{[7-propyl-3-(trifluoromethyl)-1,2-benzisoxazol-6-yl]oxy}propyl)amino]-1,3-thiazol-5-yl}acetic acid,
(25) [1-(3-{[7-propyl-3-(trifluoromethyl)-1,2-benzisoxazol-6-yl]oxy}propyl)-1H-indol-5-yl]acetic acid,
(26) [1-(3-{[7-propyl-3-(trifluoromethyl)-1,2-benzisoxazol-6-yl]oxy}propyl)-1H-benzimidazol-5-yl]acetic acid,
(27) 2-methyl-2-[1-(3-{[7-propyl-3-(trifluoromethyl)-1,2-benzisoxazol-6-yl]oxy}propyl)-1H-indol-5-yl]propanoic acid,
(28) 2-[1-(3-{[7-propyl-3-(trifluoromethyl)-1,2-benzisoxazol-6-yl]oxy}propyl)-1H-indol-5-yl]propanoic acid,
(29) [1-(3-{[7-propyl-3-(trifluoromethyl)-1,2-benzisoxazol-6-yl]oxy}propyl)-1H-1,2,3-benzotriazol-5-yl]acetic acid,
(30) [2-oxo-1-(3-{[7-propyl-3-(trifluoromethyl)-1,2-benzisoxazol-6-yl]oxy}propyl)-2,3-dihydro-1H-indol-5-yl]acetic acid
(31) [1-(3-{[7-propyl-3-(trifluoromethyl)-1,2-benzisoxazol-6-yl]oxy}propyl)-1H-indazol-5-yl]acetic acid,
(32) 6-chloro-1-(3-{[7-propyl-3-(trifluoromethyl)-1,2-benzisoxazol-6-yl]oxy}propyl)-1H-indole-5-carboxylic acid,
(33) 4-chloro-1-(3-{[7-propyl-3-(trifluoromethyl)-1,2-benzisoxazol-6-yl]oxy}propyl)-1H-indole-5-carboxylic acid,
(34) [6-chloro-1-(3-{[7-propyl-3-(trifluoromethyl)-1,2-benzisoxazol-6-yl]oxy}propyl)-1H-indol-5-yl]acetic acid,
(35) [6-chloro-2-oxo-1-(3-{[7-propyl-3-(trifluoromethyl)-1,2-benzisoxazol-6-yl]oxy}propyl)-2,3-dihydro-1H-indol-5-yl]acetic acid,
(36) [4-chloro-1-(3-{[7-propyl-3-(trifluoromethyl)-1,2-benzisoxazol-6-yl]oxy}propyl)-1H-indol-5-yl]acetic acid,
(37) 6-methoxy-1-(3-{[7-propyl-3-(trifluoromethyl)-1,2-benzisoxazol-6-yl]oxy}propyl)-1H-indole-5-carboxylic acid, and
(38) 3-[1-(3-{[7-propyl-3-(trifluoromethyl)-1,2-benzisoxazol-6-yl]oxy}propyl)-1H-indol-5-yl]propanoic acid; or a pharmaceutically acceptable salt thereof.

7. A pharmaceutical composition comprising a compound of claim 1, or a pharmaceutically acceptable salt thereof, and a pharmaceutically acceptable carrier.

8. The use of a compound of claim 1, or a pharmaceutically acceptable salt thereof, for the manufacture of a medicament useful for the treatment of a disease mediated by the LXR receptor in a human patient in need of such treatment.

9. A method for the treatment of one or more diseases selected from the group consisting of dyslipidemia, atherosclerosis, hyperlipidemia, hypercholesterolemia, low HDL, and high LDL, comprising the administration of a therapeutically effective amount of a compound of claim 1, or a pharmaceutically acceptable salt thereof, to a patient in need thereof.

10. A method for reducing the risk of the development of atherosclerosis in a patient at risk of developing atherosclerosis by administering a prophylactically effective amount of a compound of claim 1, or a pharmaceutically acceptable salt thereof, to said patient.

11. A pharmaceutical composition comprising a compound of claim 1, or a pharmaceutically acceptable salt thereof, a pharmaceutically acceptable carrier, and a second pharmaceutical compound or a pharmaceutically acceptable salt thereof, selected from the group consisting of:
a) HMG-CoA reductase inhibitors;
b) HMG-CoA synthase inhibitors;
c) Squalene epoxidase inhibitors;
d) Squalene synthetase inhibitors;
e) Acyl-coenzyme A:cholesterol acyltransferase (ACAT) inhibitors
f) Microsomal triglyceride transfer protein (MTP) inhibitors;
g) Cholesteryl ester transfer protein (CETP) inhibitors;
h) Niacin;
i) Probucol;
j) Bile acide sequestrants;
k) LDL receptor inducers;
l) Platelet aggregation inhibitors;
m) Human peroxisome proliferator activated receptor gamma (PPARγ) agonists and partial agonists;
n) PPARα agonists;
o) PPAR dual α/γ agonists;
p) Vitamin B6;
q) Vitamin B12;
r) Folic acid;
s) Anti-oxidant vitamins;
t) Beta-blockers;
u) Angiotensin II antagonists;
v) Angiotensin converting enzyme inhibitors;
w) Calcium channel blockers;
x) Endothelin antagonists;
y) Agents that enhance ABCA1 gene expression
z) FXR ligands;
aa) Bisphosphonate compounds;
bb) Cyclooxygenase inhibitors; and
cc) Cholesterol absorption inhibitors.

12. A pharmaceutical composition comprising a compound of claim 1, or a pharmaceutically acceptable salt thereof, a pharmaceutically acceptable carrier, and a second pharmaceutical compound or a pharmaceutically acceptable salt thereof, selected from the group consisting of:
a) simvastatin;
b) pravastatin;
c) fluvastatin;
d) atorvastatin;
e) pitavastatin;
f) rosuvastatin;
g) lovastatin;
h) torcetrapib;
i) niacin;
j) probucol;
k) pioglitazone;
l) rosiglitazone;
m) muraglitazar;
n) tesaglitazar;
o) naveglitazar;
p) Tak-559;
q) clofibrate;
n) fenofibrate;
o) gemfibrozil;
p) losartan;
q) enalapril;
r) captopril;
s) nifedipine;
t) diltiazam;
u) alendronate sodium;
v) rofecoxib;
w) celecoxib;
x) etoricoxib;
y) ezetimibe;
z) netoglitazone; and
aa) balaglitazone.
